(19) United States
(12) Patent Application Publication
(10) Pub. No.: US 2026/0298828 A1
(43) Pub. Date: Oct. 1, 2026

(54) METHODS OF ACQUIRING MICROSCOPIC IMAGES OF BIOLOGICAL SAMPLES

(71) Applicant: Yale University, New Haven, CT (US)

(72) Inventors: Antonio Giraldez, New Haven, CT (US); Mark Pownall, New Haven, CT (US)

(21) Appl. No.: 19/480,886

(22) PCT Filed: May 3, 2024
(86) PCT No.: PCT/US2024/027637

§ 371 Date: Nov. 3, 2025

Related U.S. Application Data
(60) Provisional application No. 63/500,193, filed on May 4, 2023.

(51) Int. Cl. G01N 21/64; G01N 1/30; G01N 1/36

(52) U.S. Cl. CPC G01N 21/6458; G01N 1/30; G01N 1/36; G01N 2021/6439

(57) ABSTRACT

Described herein is a method of acquiring a microscopic image of a biological sample. In certain embodiments, the method includes at least one of the following: the method including: embedding the biological sample in a first swellable hydrogel; expanding the first swellable hydrogel and the biological sample embedded therein to obtain a first expanded biological sample; embedding the first expanded biological sample in a second swellable hydrogel; labeling a DNA molecule in a chromatin in the biological sample with a first dye; expanding the second swellable hydrogel and the first expanded biological sample embedded therein to obtain a second expanded biological sample; acquiring the microscopic image of the second expanded biological sample with a light microscopy technology. Also described is a method of evaluating the effect of a treatment of a biological sample using the imaging method.

### CROSS-REFERENCE TO RELATED APPLICATIONS

The present application claims priority under 35 U.S.C. § 119(e) to U.S. Provisional Patent Application No. 63/500,193, filed May 4, 2023, which is incorporated herein by reference in its entirety.

### STATEMENT REGARDING FEDERALLY SPONSORED RESEARCH

This invention was made with government support under R35GM122580 and F31HD104443 awarded by National Institutes of Health. The government has certain rights in the invention.

### BACKGROUND

Visualizing chromatin structures with light microscopy technologies would allow the study of nuclear function, such as DNA damage and repair. DNA replication, epigenetic control of gene expression, and genome structure. Such visualization would also allow one to understand how sequence variation affects chromatin structure and function in disease states.

Our ability to visualizing chromatin structures, however; is hampered by the image resolutions in conventional light microscopy, which is limited to half of the wavelength of the light being used.

Accordingly, there is a need for novel light microscopy technologies that can visualize the structures of chromatin or associated molecules with higher image resolutions. The present invention addresses this need.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIGS. 1A-1G demonstrate that Nanog forms DNA-bound foci associated with zygotic transcription, in accordance with some embodiments. FIG. 1A: Schematic detailing how Nanog and miR-430 transcription were visualized in living embryos. FIG. 1B: Live imaging shows Nanog foci associated with miR-430 transcription at 2.5 hpf. n=10 nuclei. FIG. 1C: Live imaging shows Nanog foci are formed and disappear prior to Pol II elongation at 2.75 hpf n=2 nuclei, 1 embryo. FIG. 1D: Quantification of fluorescence intensity in FIG. 1C. FIG. 1E: Schematic showing the process of ChromExM and expected results. AA, acrylamide; SA, sodium acrylate, DHEBA, N,N′-(1,2-dihydroxyethylene)bisacrylamide; BIS, N,N′-methylenebis(acrylamide). FIG. 1F: Unexpanded and expanded nuclei from two unrelated embryos stained for DNA and imaged with a 10×0.3 NA objective demonstrate the enhanced resolution provided by ChromExM. n>100 nuclei, >30 embryos. FIG. 1G: Quantification of the nuclear expansion factor determined by measuring the nuclear cross-sectional area. n=171 unexpanded nuclei from 3 embryos and 104 expanded nuclei from 17 embryos.

FIGS. 2A-2G demonstrate that ChromExM preserves chromatin architecture and resolves chromatin fibers and individual nucleosomes, in accordance with some embodiments. FIG. 2A: Schematic showing how the chromatin is painted with photocleaved stripes to detect perturbations after expansion. FIG. 2B: Expanded nucleus showing that photocleaved stripes remain parallel and sharp after expansion. The image is a maximum intensity projection of several z-slices. n=20 nuclei from 3 embryos. FIG. 2C: Quantification of relative inter-strip distance in photocleaved stripes versus a simulated control (Methods) shows minimal variation in the spacing between stripes after expansion. n=3 nuclei from 3 embryos. FIG. 2D: Schematic of in vitro assembled nucleosome arrays. FIG. 2E: ChromExM image (left) showing nucleosome arrays with H3 staining and Electron Microscopy image of nucleosome arrays with a similar conformation as the expanded array. FIG. 2F: ChromExM image with metabolic DNA labelling at 2.75 hpf showing individual chromatin fibers and a schematic of chromatin. n=6 nuclei, 5 embryos. FIG. 2G: H3 staining imaged with confocal and STED resolves individual nucleosomes. n=468 nucleosomes; 3 nuclei from 2 embryos.

FIGS. 3A-3U: Visualizing the nanoscale organization Nanog, nucleosomes, Pol II, and transcription, in accordance with some embodiments. FIGS. 3A-3D: Representative images showing Nanog and H3 staining at 4 hpf. FIG. 3B: represents Class 1 organization. FIG. 3C: represents the white boxed region in FIG. 3A and shows Class 2 organization. FIG. 3D represents the green boxed region in FIG. 3A and shows Class 3 organization. n=3 nuclei from 2 embryos. FIGS. 3E-3G: Box plots quantifying the percentage of H3 particles in each class of Nanog-H3 interactions. The median is denoted by the orange line. n=104,347 distances. FIGS. 3H-3K: Representative images showing Nanog and Pol II pSer5 staining at 4 hpf, n=7 nuclei from 2 embryos. FIG. 3I represents the green boxed region in FIG. 3H and shows Class 1 organization; areas indicated by arrowheads are magnified to the right. FIG. 3J represents Class 2 organization. FIG. 3K represents the magenta boxed region in FIG. 3H and shows Class 3 organization; arrowheads indicate regions enriched for Nanog (open arrowhead) and Pol II pSer5 (closed arrowhead). FIG. 3L: Density plot of the distance to nearest neighbor for Nanog and Pol II pSer5 particles; n=169,866 distances. FIG. 3M: Box plots quantifying the number of Pol II pSer5 particles in Class 1 (n=36) and Class 2 strings (n=5,682). FIG. 3N: Box plots quantifying the number of Nanog particles in Class 1 and Class 2 Pol II pSer5 strings. FIG. 3O: Box plots quantifying the length of Pol II pSer5 strings in Class 1 and Class 2. FIGS. 3P-3Q: ChromExM images showing Pol II pSer5 and miR-430 RNA detected by HCR RNA FISH at 4 hpf. FIG. 3Q represents the boxed region in FIG. 3P. FIGS. 3R-3U: Representative images showing nascent RNA and Pol II pSer5 at 4 hpf n=7 nuclei from 3 embryos. Arrowheads in FIG. 3R indicate a central Pol II pSer5 hub (closed arrowhead) with an emanating string associated with nascent transcripts (open arrowhead). Arrowheads in S indicate individual Pol II pSer5 particles.

FIGS. 4A-4P demonstrate that Nanog-bound enhancers and Pol II-bound promoters are kicked apart by transcription elongation, in accordance with some embodiments. FIGS. 4A-4B: Representative images of Nanog and Pol II pSer5 in DMSO treated embryos at 4 hpf. n=6 nuclei from 2 embryos. FIGS. 4C-4D: Visualization of Pol II pSer5 strings identified in DMSO treated embryos at 4 hpf. FIGS. 4E-4F: Representative images of Nanog and Pol II pSer5 in α-amanitin treated embryos. FIGS. 4G-4H: Visualization of Pol II pSer5 strings identified in α-amanitin treated embryos. FIG. 4I: Quantification of the number of Pol II pSer5 macroclusters detected in DMSO and α-amanitin treated embryos. P=0.0304: unpaired t test. FIG. 4J: Quantification of total Pol II pSer5 string length per nucleus in DMSO and α-amanitin treated embryos. P=0.0002; unpaired t test. FIG. 4K: Density plot of the distance to nearest neighbor for Nanog to Pol II pSer5 particles in DMSO and α-amanitin treated embryos. n=348,211 and 229,706 distances, respectively. P<0.001; Mann-Whitney U test. FIG. 4L: Histogram showing the number of Pol II pSer5 particles within 200 nm of each Nanog particle in DMSO and α-amanitin treated embryos. P<0.001; Mann-Whitney U test. FIG. 4M: Line plot showing Pol II pSer5 binding across gene bodies+/−2 kb at zygotic genes in wild-type (WT) and α-amanitin treated embryos. FIG. 4N: Representative genome tracks of pan-Pol II, Pol II pSer5, and Nanog binding showing accumulation of Pol II pSer5 at the promoter and Nanog-bound enhancers in the presence of α-amanitin. FIG. 4O: Heatmaps showing Pol II pSer5 binding at Nanog-bound accessible regions in WT and α-amanitin treated embryos. Regions are ranked by Nanog ChIP-seq signal. FIG. 4P: Schematic showing the kiss-and-kick model.

FIGS. 5A-5I demonstrate that Nanog forms DNA-bound foci associated with zygotic transcription, in accordance with some embodiments. FIG. 5A: Live imaging shows Nanog foci forming at the 32-cell stage, prior to the activation of miR-430 transcription, and associating with miR-430 transcription at the 64- and 512-cell stage. FIG. 5B: Quantification of the fluorescence intensity enrichment in Nanog foci at the 32-, 64-, 256-, and 512-cell stages. FIG. 5C: Representative images of the transgenic line used for llama-tag imaging showing that nuclear signal and nuclear foci depend on the presence of Nanog-llama. Uninjected transgenic embryos show no nuclear enrichment for GFP. Embryos injected with a 3× nuclear localization signal (NLS)-llama tag show nuclear enrichment but do not form foci. FIG. 5D: Representative fixed images (maximum projection) showing that Pol II pSer2 foci (arrow heads) are lost in MZnanog−/− embryos at 2.5 hpf. N≥2 embryos. FIG. 5E: Representative fixed images (maximum projection) and quantification showing that Pol II pSer5 levels are significantly reduced and foci (arrow heads) are lost in MZnanog−/− embryos compared to wild-type embryos at 2.5 hpf. P=0.0058, unpaired t test; N≥18 nuclei from 3 embryos. FIG. 5F: Schematic of the Nanog DNA-binding mutant (DBM) constructs used for live imaging. Nanog-DBMa contains ten alanine substitutions in the homeodomain predicted to prevent all DNA-binding activity. Nanog-DBMs contains three alanine substitutions in the homeodomain predicted to prevent sequence-specific DNA binding activity. FIG. 5G: Live imaging shows that both Nanog-DBM constructs fail to form foci at 3 hpf FIG. 5H: Western blot showing that all Nanog constructs used for live-imaging are expressed at similar levels. FIG. 5I: Nanog-DBM constructs tagged with 6×-Myc instead of mEmerald are unable to rescue the phenotype of MZnanog—embryos at 6 hpf which fail to gastrulate. This confirms that the Nanog-DBM constructs are not functional.

FIGS. 6A-6D demonstrate that ChromExM preserves chromatin architecture, in accordance with some embodiments. FIG. 6A: Unexpanded nucleus showing photocleaved stripes as in FIG. 2B. FIG. 6B: Line plot of Neutravidin 550 intensity along the dashed line in FIG. 6A shows changes in intensity corresponding to the photocleaved stripes. FIG. 6C: Example images demonstrating how the inter-stripe distance was quantified for results shown in FIG. 2C. FIG. 6D: Quantification of the standard deviation in inter-stripe distance for real and simulated photocleaved stripes shows that there is no significant change in the variation of inter-stripe distance in photocleaved stripes when compared to simulated stripes. P=0.0630; unpaired t test. n=3 nuclei from 3 embryos.

FIGS. 7A-7L demonstrate that ChromExM preserves nucleosome-scale chromatin organization and resolves chromatin fibers and individual nucleosomes, in accordance with some embodiments. FIG. 7A: Schematic of using FRAP to estimate the gel mesh size. FIG. 7B: Quantification of FRAP for different sized molecules. n=3. FIGS. 7C-7E: Quantification of FRAP for each molecule plotted with measurements from unbleached control areas. FIG. 7F: Single-channel images of ChromExM performed on in vitro assembled nucleosome arrays stained for H3 and DNA (SYTOX Green) as shown in FIG. 2E. FIG. 7G: Additional ChromExM images of nucleosome arrays. FIG. 7H: Quantification of the number of nucleosomes detected in each nucleosome array. n=7 arrays by ChromExM and 10 arrays by EM. FIG. 7I: Quantification of the relative frequency of nucleosomes detected with H3 staining and confocal imaging that were confirmed to be individual nucleosomes by STED imaging. n=468 H3 spots. FIG. 7J: Quantification of the change in intensity for nuclei co-labelled SYTOX Green and f-ara-EdU detected with AZ555 picolyl azide shows that AZ555 is more photostable than SYTOX Green for imaging expanded nuclei. FIG. 7K: Unexpanded images showing metabolic DNA labeling with f-ara-EdU detected with either AZ555 azide of AZ555 picolyl azide. Non-linear contrast adjustments were made for visualization purposes in the images denoted by Gamma. FIG. 7L: Quantification of FIG. 7I shows that AZ555 picolyly azide detection provides ~45-fold more intense nuclear staining than AZ555 azide. P<0.001; unpaired 1 test. n≥100 nuclei from 3 embryos.

FIGS. 8A-8I: Visualizing the nanoscale organization Nanog and nucleosomes, in accordance with some embodiments. FIGS. 8A-8C: Single-channel images of those shown in FIGS. 3B-3D. FIG. 8B, rightmost panel is a 3D render of the image shown in the left panels of FIG. 8B. FIG. 8D: Single-channel images showing Class 4 organization of Nanog and H3. FIG. 8E: Box plot quantifying the percentage of H3 particles in Class 4 of Nanog-H3 interactions. The median is denoted by the orange line. FIG. 8F: ChromExM image showing that Nanog is specifically detected in embryos injected with Nanog-6xMyc mRNA. FIG. 8G: Quantification of Nanog particles in Nanog-6xMyc injected versus uninjected embryos shows a false detection rate of 0.05%. n=3 uninjected nuclei (1 embryo) and 4 injected nuclei (2 embryos). FIG. 8H: Schematic showing how the nearest neighbor distance (point-to-set correlation) between Nanog and H3 particles was measured. FIG. 8I Density plot showing the nearest-neighbor distance between Nanog and H3 particles. Distance criteria used to define Class 1-4 organization are shaded on the graph. n=104,347 distances from 3 nuclei across 2 embryos.

FIGS. 9A-9H: Visualizing the nanoscale organization Nanog and Pol II. FIG. 9A: Histogram of Pol II pSer5 particle intensities. n=192,521 particles. FIG. 9B: Histogram of Pol II pSer5 particle sizes. n=192,521 particles. FIG. 9C: Histogram of Nanog particle intensities. n=169,866 particles. FIG. 9D: Histogram of Nanog particle sizes. n=169,866 particles. FIGS. 9E-9G: Single-channel images of those shown in FIG. 3 I-K. FIG. 9H Representative example of how Pol II pSer5 strings are identified and visualized.

FIGS. 10A-10F: Single-haploid embryo genome assembly by long-read sequencing, in accordance with some embodiments. FIG. 10A Graphs of unitigs from one wild-type (WT) sample. Regions containing mature miR-430 genes are colored red. FIG. 10B: Unitig graph of the single graph containing mature miR-430 genes. FIG. 10C: Bar graph showing the number of mature miR-430 genes detected on all linear contigs in WT and miR-430^{-/-} samples. FIG. 10D: Bar graph showing the number of mature miR-430 genes detected in reads from WT and miR-430^{-/-} samples after normalization to single-copy orthologs (Methods). FIG. 10E Genome track of the only contig (ptg0000881; total length ~3 Mb) containing mature miR-430 genes showing the alignment of uniquely mapped reads only. Reads are shown as blue bars. Red lines connect primary and supplementary alignments of the same read indicating large gaps in the read alignment. FIG. 10F: Genome track showing the region of GRCzI 1 chromosome 10 with annotated miR-430 genes. No reads mapping to this region contain miR-430 genes.

FIGS. 11A-11H: ChromExM visualizes the nanoscale organization miR-430 transcription, in accordance with some embodiments. FIG. 11A: Genome track of pgt0000881 showing Pol II pSer5 and Nanog ChIP-Seq, CAGE-Seq, and miR-430 RNA FISH probes. FIG. 11B: Zoom in of the region above the magenta line in FIG. 11A. FIG. 11C: Unexpanded imaging of miR-430 RNA FISH in wild-type (WT) and miR-430^{-/-}embryos shows that HCR RNA FISH signal is specific to miR-430. n≥100 nuclei from 3 embryos. FIG. 11D: Single channel images of those shown in FIGS. 3P and Q showing that Class 1 Pol II pSer5 strings correspond to the transcriptionally active miR-430 locus. Lower panel shows that miR-430 FISH signal is lost in miR-430^{-/-} embryos but Class 2 Pol II pSer5 strings are still observed. FIG. 11E: Additional images showing a more compacted organization of the miR-430 locus at 4 hpf. FIG. 11F: Representative image of Pol II pSer5 strings identified in wild-type (WT) embryos at 4 hpf. Arrowheads indicate Class 1 strings. n=5 nuclei from 2 embryos. FIG. 11G: Representative image of Pol II pSer5 strings identified in miR-430^{-/-} embryos at 4 hpf n=7 nuclei from 2 embryos. FIG. 11H: Representative ChromExM images showing Class 2 (solid box) and Class 3 (dashed box) Pol II pSer5 structures in miR-430^{-/-} embryos at 4 hpf.

FIGS. 12A-12H: ChromExM visualizes the nanoscale organization of Nanog, Pol II, and miR-430 transcription prior to the major wave of ZGA, in accordance with some embodiments. FIGS. 12A-12D: Representative images showing Nanog and Pol II pSer5 staining at 2.5 hpf, n=6 nuclei from 2 embryos. FIG. 12B represents box 1 in FIG. 12A and shows Class 1 strings. FIG. 12C represents box 2 in FIG. 12A and shows Nanog-Pol II co-clustering. FIG. 12D represents box 3 in FIG. 12A and shows a Class 2 string. FIGS. 12E-12H: Representative images of Pol II pSer5 and miR-430 FISH staining at 2.5 hpf (n=7 nuclei from 2 embryos), showing a compacted form of the miR-430 locus (FIG. 12E) and a more extended form of the miR-430 locus (FIG. 12G). FIGS. 12F and 12H: represent the boxed regions in FIGS. 12E and 12G respectively.

FIGS. 13A-13G: ChromExM visualizes the nanoscale organization nascent transcription, in accordance with some embodiments. FIGS. 13A-13D: Single-channel images of those shown in FIG. 3R-U. FIGS. 13E-13F Additional example of Pol II pSer5 associated with EU. FIG. 13F represents the boxed region in FIG. 13E. FIG. 13G: Quantification of the mean RNA intensity in Pol II pSer5 macroclusters and strings. P>0.05; unpaired t test.

FIGS. 14A-14M: Nanog-bound enhancers and Pol II-bound promoters are kicked apart by transcription elongation, in accordance with some embodiments. FIG. 14A: Representative images of Pol II pSer2 and Pol II pSer5 in DMSO and α-amanitin treated embryos at 4 hpf n≥150 nuclei from 3 embryos. FIG. 14B: Quantification of Pol II pSer2 levels. P<0.001; unpaired t test. FIG. 14C: Quantification of Pol II pSer5 levels. P<0.001: unpaired t test. FIGS. 14D-14E: Single-channel images of those shown in FIGS. 4A-4B. FIGS. 14F-14G: Single-channel images of those shown in FIGS. 4E-4F. FIG. 14H: Bar plot showing the volume of Pol II pSer5 macroclusters detected in nuclei from DMSO and α-amanitin treated embryos. P=0.0028; unpaired t test. FIG. 14I: Bar plot showing the number of Pol II pSer5 particles detected in nuclei from DMSO and α-amanitin treated embryos. P=0.4786; unpaired t test. FIG. 14J: Density plot of the distance to nearest neighbor for Nanog to Pol II pSer5 particles in untreated and α-amanitin treated miR-430^{-/-} embryos. Median distance 127 nm vs 107 nm; P<0.001; Mann-Whitney U test. n=533,571 and 784,345 distances, respectively, from 7 nuclei across 2 embryos. FIG. 14K: Heatmaps showing Pol II pSer5 ChIP-seq and input signal across gene bodies (TSS: transcription start site: TES: transcription end site)+/−2 kb at zygotic genes in wild-type (WT) and α-amanitin treated embryos. FIG. 14L: Representative genome tracks of pan-Pol II, Pol II pSer5, and Nanog binding showing accumulation of Pol II pSer5 at the promoter and Nanog-bound enhancers in WT and α-amanitin treated embryos. FIG. 14M: Heatmaps showing Pol II pSer5 binding at active enhancers in WT and α-amanitin treated embryos.

FIGS. 15A-15C: non-limiting a system that uses single guide RNAs (sgRNAs) and Cas proteins to specifically label genes. FIGS. 15A-15B: embryos were injected with sgRNAs and mRNA encoding a catalytically inactive form of Cas9 (dCas9) targeting the miR-430 locus. At 3 hours post fertilization, embryos were fixed and subjected to ChromExM. The first expansion and the second expansion were both about 4.5×. FIG. 15C: dCas9 was detected by antibody staining in expanded cells injected with the dCas9 and the sgRNA, and the signal was absent in embryos not injected with dCas9. Two distinct foci of dCas9 signal are present in injected embryos (arrowheads), consistent with the two alleles of the miR-430 locus. Quantitative characteristics of these loci can then be extracted through 3D image analysis and renderings of the locus.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Recently, alight microscopy technology for acquiring images of proteins in their ultrastructural context, termed “pan-ExM,” was developed (M'Saad et al., Nature Communications 11, 3850 (2020)). Briefly, according to this technology, a biological sample is embedded and hybridized to a swellable polymer network, to which the proteins in the biological sample are crosslinked. This swellable polymer network is then expanded, which expands the biological sample embedded therein. The expanded biological sample is then embedded in a second swellable polymer network and expanded for the second time. In the expanded biological sample, proteins within the same ultrastructural context are separated from each other without significant changes in the relative positions of these proteins. Due to the expansion of the protein ultrastructure, this technology greatly improves the image resolutions when combined with conventional light microscopy technologies.

To the best of the inventors' knowledge, pan-ExM microscopy technology has not been applied to the study of chromatins. Indeed, it was reasoned that pan-ExM microscopy technology is unlikely to work for the imaging of chromatins, as the overall structures of chromatins are not expected to survive the expansions. Chromatins consist of DNA molecules wrapped around histone proteins. In pan-ExM microscopy technology, only proteins are crosslinked to the swellable polymer networks and the DNA molecules are not fixed to the polymer network. Also, the DNA molecules in chromatins are extremely long and are expected to “pull” chromatins structures out of alignment with the polymer cells when the polymer network expands. Furthermore, while detection signals of proteins can be amplified with primary antibody and secondary antibody (thus compensating for the weakened detection signal density caused by the expansion), detection signals from intercalating dyes commonly used to visualize DNA molecules cannot be amplified. Thus, the lowered concentration of DNA molecules in the expanded system is expected to cause issues with detections, as well.

Unexpectedly, the study described herein (“the present study”) discovered that the general architectures of chromatins in swellable hydrogels are preserved during the expansions of the polymer networks. Without wishing to be bound by theory, it is hypothesized that the positions of DNA molecules are preserved by the entanglement between the DNA molecules and the polymer network, even though the DNA is not fixed onto the polymer network. Furthermore, minor DNA breakages occur during the expansion process, which prevent the extremely long DNA chain to pull it self out of alignment with the polymer network.

In addition, the present study discovered that the DNA portions of the chromatin can be visualized by attaching dye molecules to the DNA molecule (such as attaching the dye molecules covalently), which solves the low detection signal issue, as well.

Accordingly, in some embodiments, the present invention is directed to a method of acquiring a microscopic image of a biological sample, which includes chromatin structures therein.

The ability to visualizing the structures of chromatin (as well as proteins) allows the evaluation of, for example, transcription activities, DNA replication activities, DNA damage, DNA repair, and/or DNA-protein interaction, and the like. Treatments, such as pharmaceutical compounds applied to cells and/or administered to a subject, can in some embodiments cause changes in these cellular processes. As such, visualizing chromatin structures can be used to evaluate the effects of such treatments of the biological sample.

Accordingly, in some aspects, the present invention is directed to a method of evaluating the effect of a treatment with a biologically active agent.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of ±20% or ±10%, in certain embodiments ±5%, in certain embodiments 10%, in certain embodiments ±0.1% from the specified value, as such variations are appropriate to perform the disclosed methods.

### Method of Acquiring Microscopic Image

In some aspects, the present invention is directed to a method of acquiring an image.

In some embodiments, the microscopic image is a light microscopic image.

In some embodiments, the method is a method of acquiring a microscopic image of a biological sample.

In some embodiments, the method includes embedding the biological sample in a first swellable hydrogel.

In some embodiments, the method includes expanding the first swellable hydrogel and the biological sample embedded therein to obtain a first expanded biological sample.

In some embodiments, the method includes embedding the first expanded biological sample in a second swellable hydrogel.

In some embodiments, the method includes labeling a DNA molecule in a chromatin in the biological sample with a first dye.

In some embodiments, the method includes expanding the second swellable hydrogel and the first expanded biological sample embedded therein to obtain a second expanded biological sample.

In some embodiments, the method includes acquiring the microscopic image of the second expanded biological sample with a light microscopy technology.

In some embodiments, the microscopic image includes the labeled chromatin.

Hydrogels swell because the polymer network in the hydrogel is often elastic and hydrophilic. As such, a hydrogel can absorb water to increase its volume, but not to the extent of dissolving itself, at least because the elastic tension within the networked polymer chains can maintain the structure of the hydrogel. As such, the choice of the swellable hydrogels within the present invention is not limited. Non-limiting examples of swellable hydrogels include chitosan-based hydrogels, alginate-based hydrogels, fibrin-based hydrogels, collagen-based hydrogels, gelatin-based hydrogels, polyvinylpyrrolidone (PVP)-based hydrogels, poly(N-isopropylacrylamide) (PNIPA)-based hydrogels, polyvinyl alcohol-vinyl acetate (PVA)-based hydrogels, polyacrylic acid (PAA)-based hydrogels, poly(methyl methacrylate) (PMMA)-based hydrogels, polyacrylamide (PAM)-based hydrogels, and the like.

In some embodiments, the first swellable hydrogel or the second swellable gel is a polyacrylamide-based hydrogel.

In some embodiments, embedding the biological sample in the first swellable hydrogel comprises crosslinking the biological sample to the first swellable hydrogel.

In some embodiments, the expansion of the first swellable hydrogel or the second swellable hydrogel is substantially isotropic, meaning that the expansion of the hydrogel is substantially uniform in all directions. In some embodiments, the differences between the linear expansion factors of the first swellable hydrogel or the second swellable hydrogel in any given directions are within about 40% or less, such as about 30% or less, about 25% or less, about 20% or less, about 15% or less, about 10% or less, about 7.5% or less, about 5% or less, about 2.5% or less, or about 1% or less.

In some embodiments, a first linear expansion factor of the biological sample during the expansion of the first swellable hydrogel is about 2 or more, such as about 2.5 or more, about 3 or more, about 3.5 or more, about 4 or more, about 4.5 or more, or about 5 or more.

In some embodiments, a second linear expansion factor of the first expanded biological sample during the expansion of the second swellable hydrogel is about 2 or more, such as about 2.5 or more, about 3 or more, about 3.5 or more, about 4 or more, about 4.5 or more, or about 5 or more.

In some embodiments, a combined linear expansion factor of the biological sample during the expansions of the first swellable hydrogel and the second swellable hydrogel is about 6 or more, such as about 8 or more, about 10 or more, about 12 or more, about 14 or more, about 16 or more, about 18 or more, about 20 or more, or about 25 or more.

In some embodiments, the first dye for labeling the DNA molecule in the chromatin is an intercalating DNA dye. Non-limiting examples of intercalating DNA dyes include ethidium bromide, 4′,6-diamidino-2-phenylindole (DAPI), 7-aminoactinomycin D (7-AAD), crystal violet, or the like.

In some embodiments, the DNA molecule in the chromatin is labeled with the first dye via a covalent linker.

In some embodiments, the DNA molecule is introduced with a modified nucleoside for attaching the first dye covalently. Non-limiting examples of modified nucleosides that can be introduced into DNA molecules and form covalent bonds with dyes include 2′-deoxy-2′-fluoro-5-ethynyluridine (F-ara-EdU), 5-ethynyl-2′-deoxyuridine (EdU), 5-Ethynyl-2′-deoxycytidine (5-EdC), bromodeoxyuridine (BrdU), 5-azidomethyl-2′-deoxyuridine (AmdU) or the like.

In some embodiments, the modified nucleoside is introduced into the DNA molecule metabolically.

In some embodiments, the first dye is attached to the DNA molecule via a triazole group.

In some embodiments, the first dye is attached to the DNA molecule via click chemistry.

In some embodiments, the first dye is attached to the DNA molecule by metabolically introducing a modified nucleoside including an azide group or an alkyne group, and contacting the DNA molecule with the first dye including, respectively, an alkyne group or an azide group. In some embodiments, the azide group and the alkyne group react with each other via a click reaction, thereby covalently attaching the first dye to the DNA molecule.

In some embodiments, the DNA molecule is labeled after the biological sample is embedded in the second swellable hydrogel, but before the expansion of the second swellable hydrogel.

In some embodiments, apart from the DNA molecule in the chromatin, a protein or an RNA molecule is labeled such that the protein or the RNA molecule can be visible in the microscopic image, as well.

In some embodiments, the protein or the RNA molecule is labeled after the biological sample is embedded in the second swellable hydrogel, but before the expansion of the second swellable hydrogel.

In some embodiments, the protein associated with the chromatin includes a histone, a transcription factor, a polymerase, a nuclease, and/or DNA-binding portions thereof.

In some embodiments, the protein is labeled by an antibody. In some embodiments, the antibody is attached with a dye. In some embodiments, the antibody is not attached with a dye; rather a secondary antibody attached with a dye is used to detect the primary antibody for labeling the protein.

In some embodiments, the RNA molecule is labeled by a second dye. In some embodiments, the second dye is attached to the RNA molecule via a covalent bond. In some embodiments, the RNA molecule is covalently labeled in the same manner as those detailed for the DNA molecule above, apart from that the modified nucleosides metabolically incorporated into the RNA molecules are RNA-specific, such as 5-ethynyl uridine (5-EU).

In some embodiments, the RNA molecule is a transcription product of the DNA molecule in the chromatin.

In some embodiments, the first dye, the second dye, and/or the dye attached to the antibody is/are a fluorescent dye. Non-limiting examples of fluorescent dyes include xanthene and derivatives thereof, cyanine and derivatives thereof, squaraine and derivatives thereof, Squaraine rotaxane and derivatives thereof, naphthalene and derivatives thereof, coumarin and derivatives thereof, oxadiazole and derivatives thereof, anthracene and derivatives thereof, pyrene and derivatives thereof, oxazine and derivatives thereof, acridine and derivatives thereof, arylmethine and derivatives thereof, tetrapyrrole and derivatives thereof, quantum dots, or the like.

In some embodiments, the light microscopy technology used to acquire the image is a confocal microscopy technology, a stimulated emission depletion (STED) microscopy, and the like.

In some embodiments, the resolution of the acquired microscopic image is about 40 nm or better, such as about 30 nm or better, about 25 nm or better, about 20 nm or better, about 16 nm or better, about 14 nm or better, about 12 nm or better, about 10 nm or better, about 8 nm or better, about 6 nm or better, about 4 nm or better, about 3 nm or better, or about 2 nm or better, when determined from the original size of the biological sample (rather than the sizes after the expansions).

In some embodiments, the microscopic image acquired by the method herein is a 3D image. It is worth noting that the method herein can sometimes achieve resolutions close to those achieved by electron microscopy (EM). Unlike EM, however, the method herein can obtain 3D images, such as with the confocal technology.

In some embodiments, the method herein is used to detect one or more sequences in the DNA molecule. While the expansion steps sometimes cause breakages in the DNA molecule, the chance of the breakages happen to disrupt the sequences to be detected is not high in one sample. Furthermore, even if the DNA breakages happen to disrupt one or more of the sequences to be detected, such error can nonetheless be corrected by analyzing multiple samples.

In some embodiments, the one or more sequences in the DNA molecule is labeled with a probe that binds to the one or more sequences specifically. This label can either be detectable directedly by the microscopic technique (e.g., the probe comprises a detectable motif such as a fluorescent motif), or be detected by a detecting binding partner that binds to the probe (e.g., an antibody-fluorescent motif conjugate that specifically detects the probe).

In some embodiments, the probe comprises a nucleic acid and/or a protein. In some embodiments, the nucleic acid specifically binds to the one or more sequences in the DNA molecule, such as via hybridization. In some embodiments, the protein binds to the one or more sequences in the DNA molecule directly. In some embodiments, the protein binds to the nucleic acid, which binds specifically and directly to the DNA molecule. In some embodiments, the protein binds to a complex formed by the specific binding between the nucleic acid and the one or more sequences of the DNA molecule, such as by recognizing a region in the nucleic acid.

In some embodiments, the probe comprises: a single guide RNA (sgRNA) that binds to the one or more sequences specifically; and a Cas protein (such as Cas9) that binds to a complex formed by the sgRNA and the one or more sequences. In some embodiments, the Cas protein is a catalytically dead Cas protein (such as dCas9).

In some embodiments, the one or more sequences are directly visualized by a hybridization chain reaction (HCR) in situ hybridization method.

In some embodiments, detecting the one or more sequences in the DNA molecule comprises detecting the presence, absence and/or copy number of a gene, such as in the DNA molecule, in a nucleus, in a cell organelle (e.g., a mitochondrion, a ribosome, a chloroplast), in a cell, and/or in a sample.

### Method of Evaluating Effect of Treatment

In some aspects, the present invention is directed to a method of evaluating effect of a treatment on a biological sample.

In some embodiments, the treatment is a treatment to a cell, a tissue, an organ, and/or a subject (such as an animal, a mammal, or a human).

In some embodiments, the treatment is the contacting and/or administration of a compound or a composition, such as a pharmaceutical compound or pharmaceutical composition, to a cell, a tissue, an organ, and/or a subject.

In some embodiments, the method herein evaluates a change (or lack thereof) of a chromatin structure, a transcription activity, a DNA replication activity, a DNA damage, a DNA repair, a DNA-protein interaction, and/or the like, such as in response to the treatment.

In some embodiments, the method includes preparing a first biological sample and a second biological sample.

In some embodiments, the method includes subjecting the first biological sample to the treatment without subjecting the second biological sample to the treatment.

In some embodiments, the method includes acquiring a first microscopic image of the first biological sample and a second microscopic image of the second biological sample.

In some embodiments, the method includes comparing the first microscopic image with the second microscopic image.

In some embodiments, the first biological sample and the second biological sample are from the same source (e.g., from the same subject, the same tissue, the same cell line, etc.) or comparable source (e.g., from different test subjects, different subjects in the same clinical trial, etc.).

In some embodiments, subjecting the first biological sample to the treatment does not require that the first biological sample to receive the treatment directly. For example, in the case when the biological samples are from subjects, a biological sample obtained from a subject that received the treatment is considered subjected to the treatment.

In some embodiments, the first microscopic image and the second biological sample are acquired with the image acquisition methods herein, such as those described in the “Method of Acquiring Microscopic Image” section.

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1: Chromatin Expansion Microscopy Reveals Nanoscale Organization of Transcription and Chromatin (In Vivo Nanoscale Imaging with Chromatin Expansion Microscopy Reveals Principles of Pioneer-Factor Controlled Transcription)

The organization of chromatin and transcription factors in the nucleus is essential for gene expression. Zygotic genome activation (ZGA) occurs during early embryonic development and involves substantial chromatin reprogramming. However, the organization of factors within the nucleus driving this process remain unclear. Here, the present study developed chromatin expansion microscopy (ChromExM) to visualize chromatin, transcription, and transcription factors in vivo. Applying ChromExM to zebrafish embryos during ZGA revealed how the pioneer factor Nanog interacts with nucleosomes and transcribing RNA Polymerase II (Pol II), which forms string-like nanostructures associated with nascent transcripts, providing direct visualization of transcriptional elongation. In the absence of elongation, more Pol II particles clustered around Nanog, with Pol II stalled at promoters and Nanog-bound enhancers, indicating that enhancer-promoter contacts are transient and released by transcriptional elongation. The results provide a new model of enhancer-promoter interaction called kiss-and-kick and demonstrate that ChromExM is broadly applicable to study nanoscale organization within the nucleus.

### Example 2: Development and Validation of the Chromatin Electron Microscopy and Tomography (ChromEMT)

Upon fertilization, embryos undergo transcriptional and cellular reprogramming to form a totipotent zygote. This reprogramming results in the transcriptional activation of genes required to initiate zygotic development. During this process, pioneer factors open the chromatin, recruit RNA Polymerase II (Pol II), and activate transcription. However, the molecular organization of these factors during genome activation remains unclear. Nanoscale visualization of transcription and chromatin dynamics using current super-resolution approaches is limited by their ability to analyze nanoscale structure at the whole-nucleus scale, particularly in in vivo systems. Alternatively, chromatin electron microscopy and tomography (ChromEMT) provides nanometer resolution but lacks the multi-label imaging required to identify specific regulatory interactions in vivo. To overcome these limitations, the present study combined the pan-expansion microscopy (pan-ExM) super-resolution technique (O. M'Saad et al., 2020. Nature Communications 11) with multi-modal protein, RNA, and DNA labeling to resolve the nanoscale organization of chromatin, transcriptional activators, and transcription in a method termed ChromExM herein. The present study applied this approach to uncover how the pioneer factor Nanog interacts with chromatin and recruits Pol II to activate transcription during genome reprogramming after fertilization.

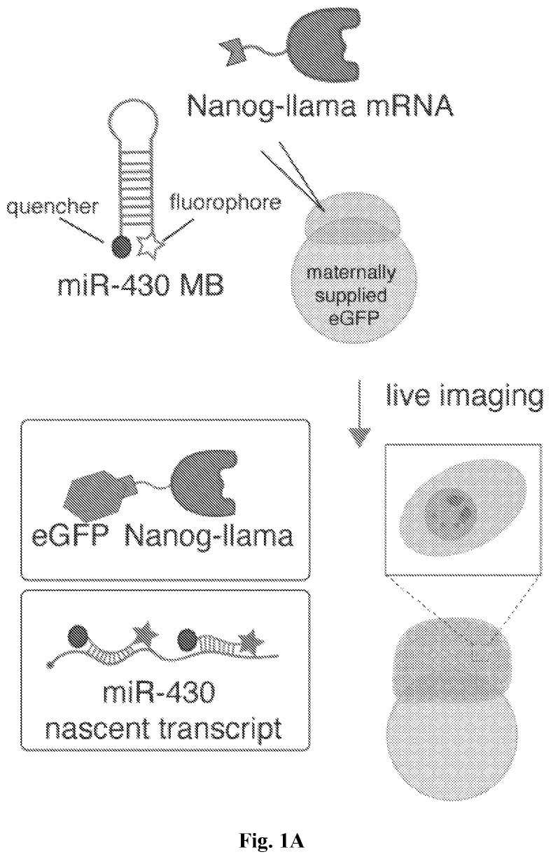

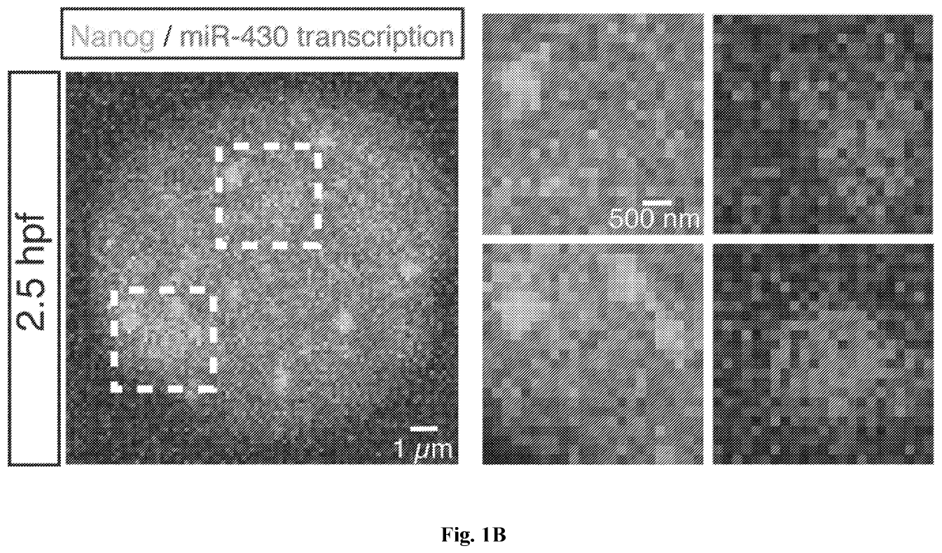

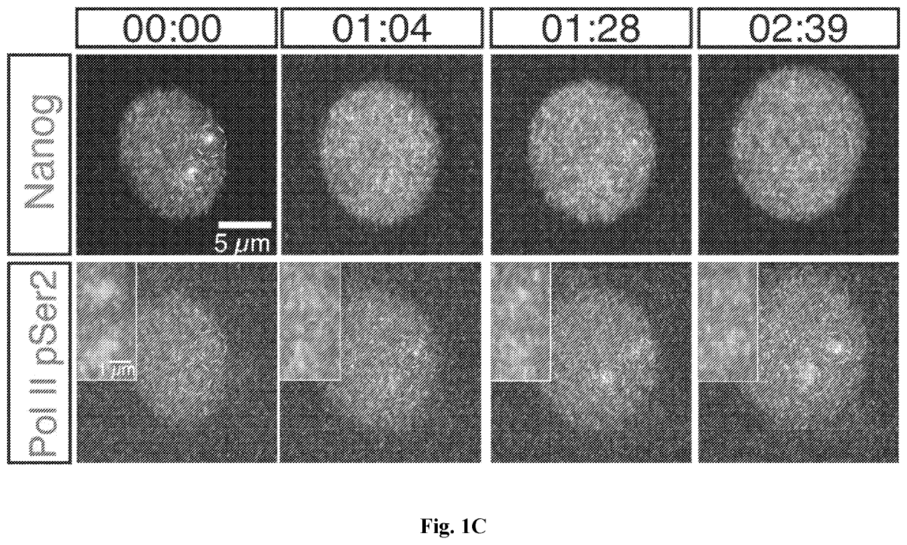

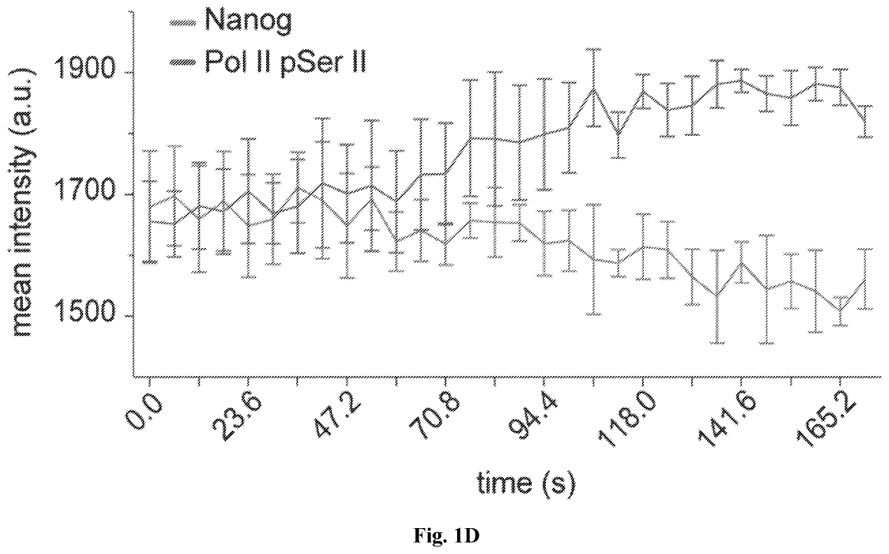

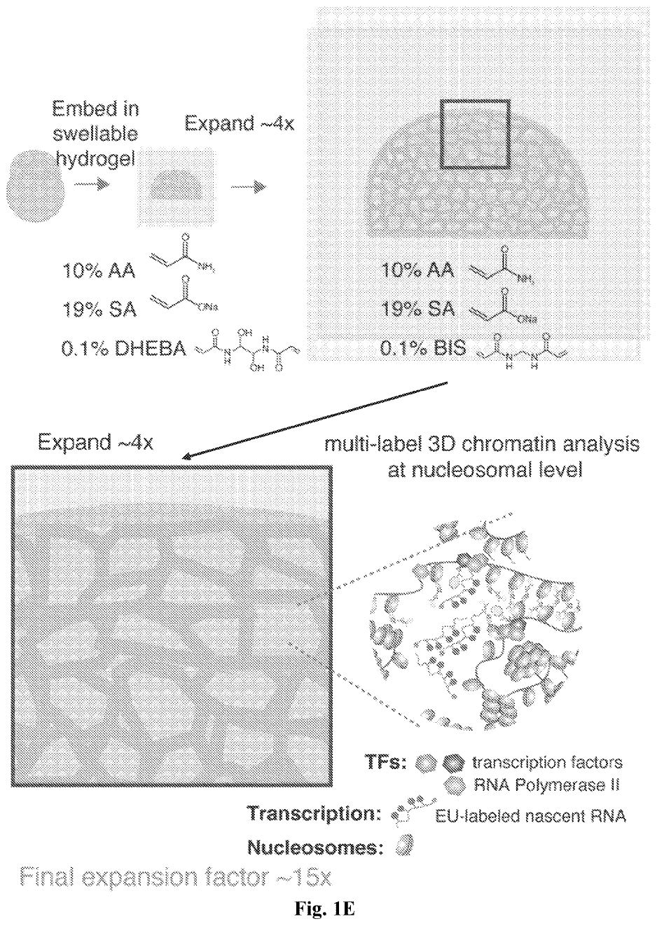

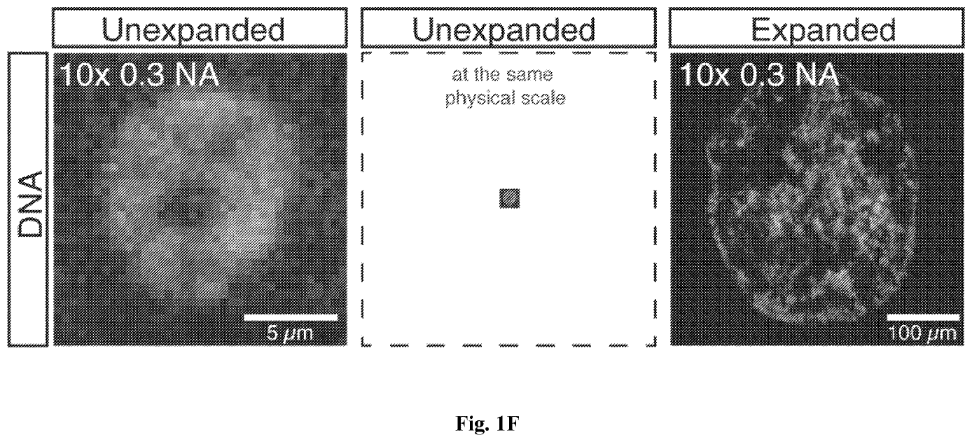

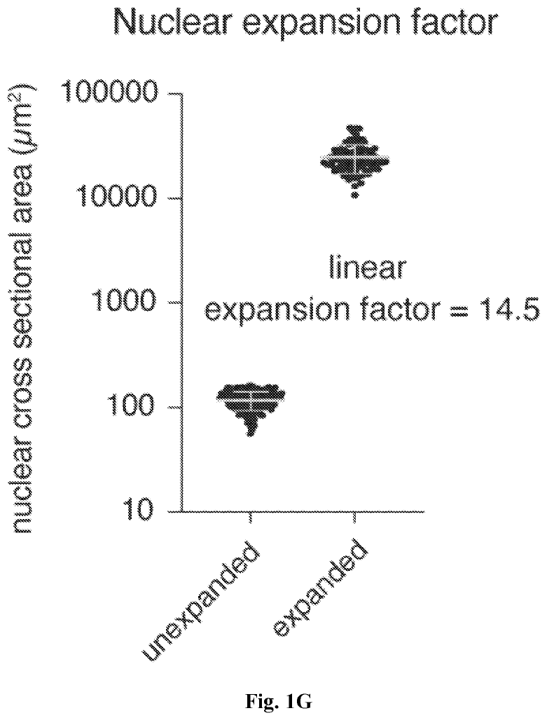

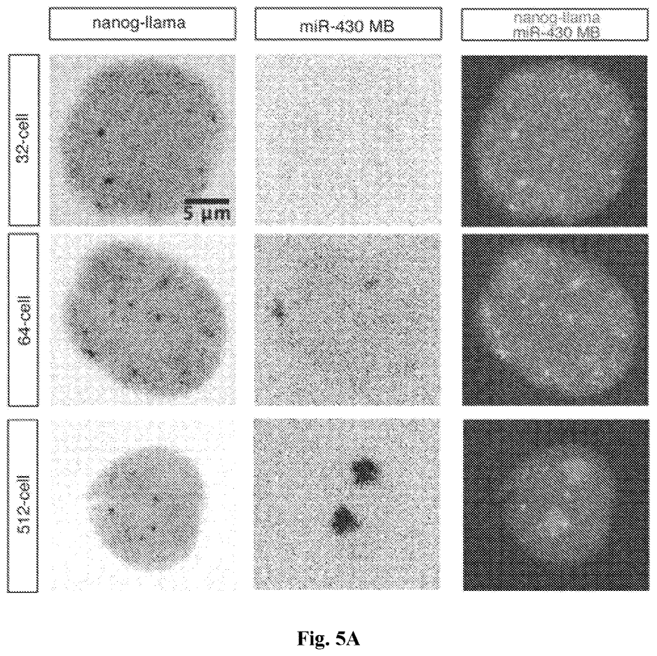

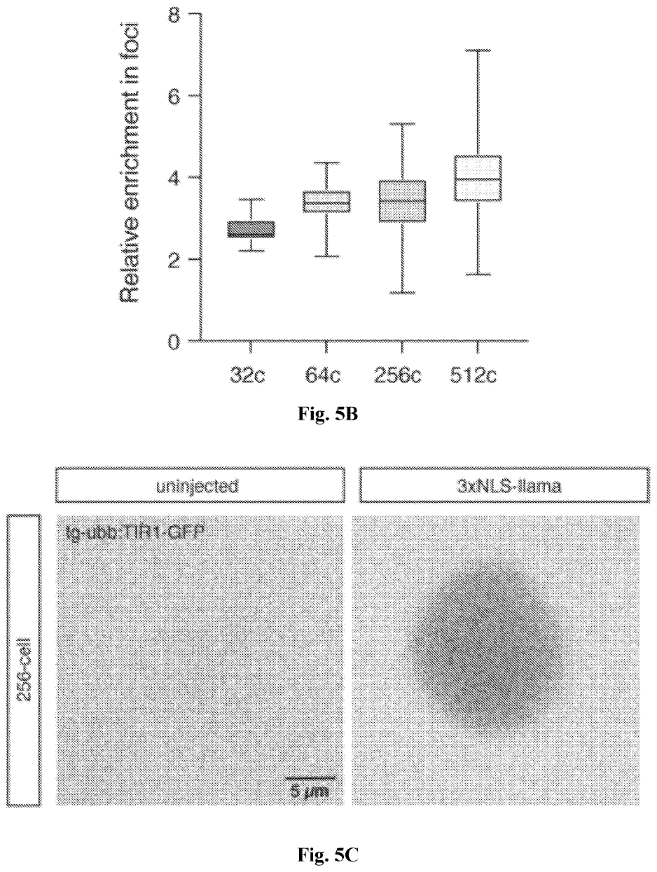

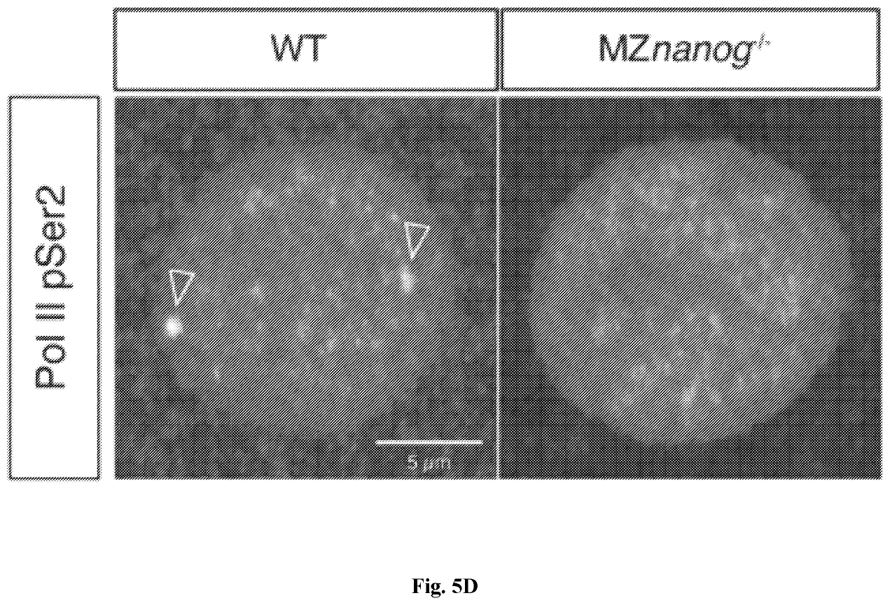

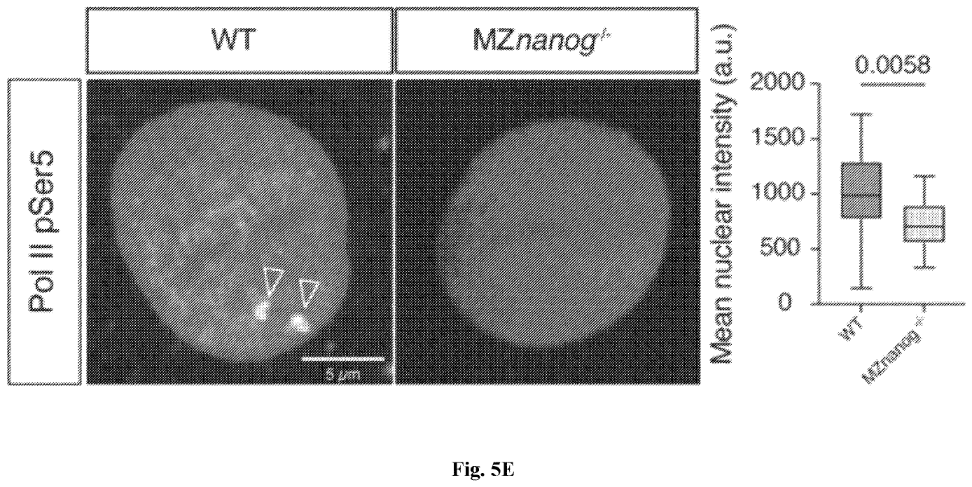

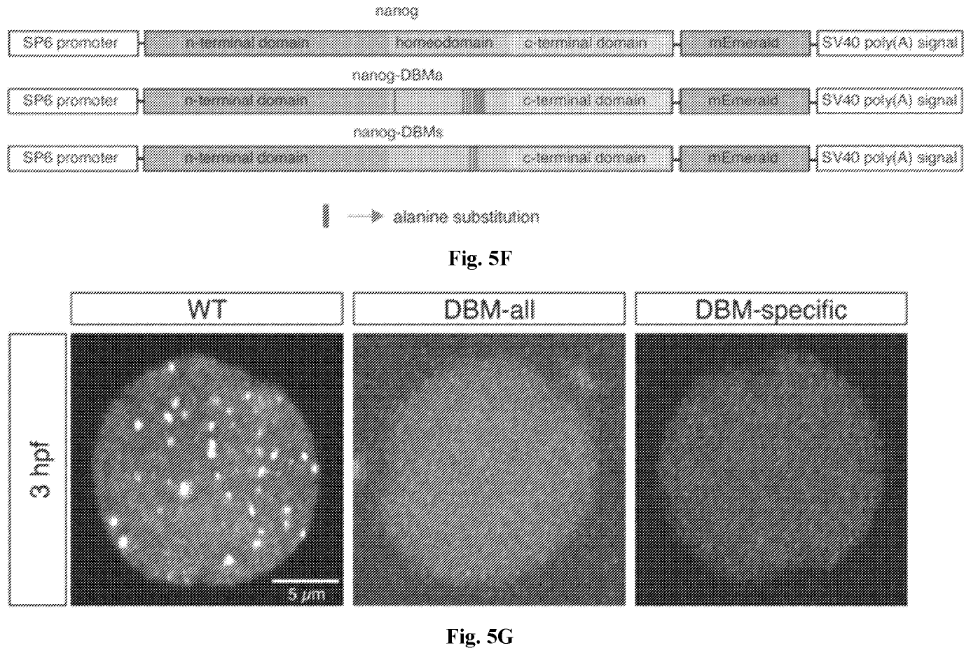

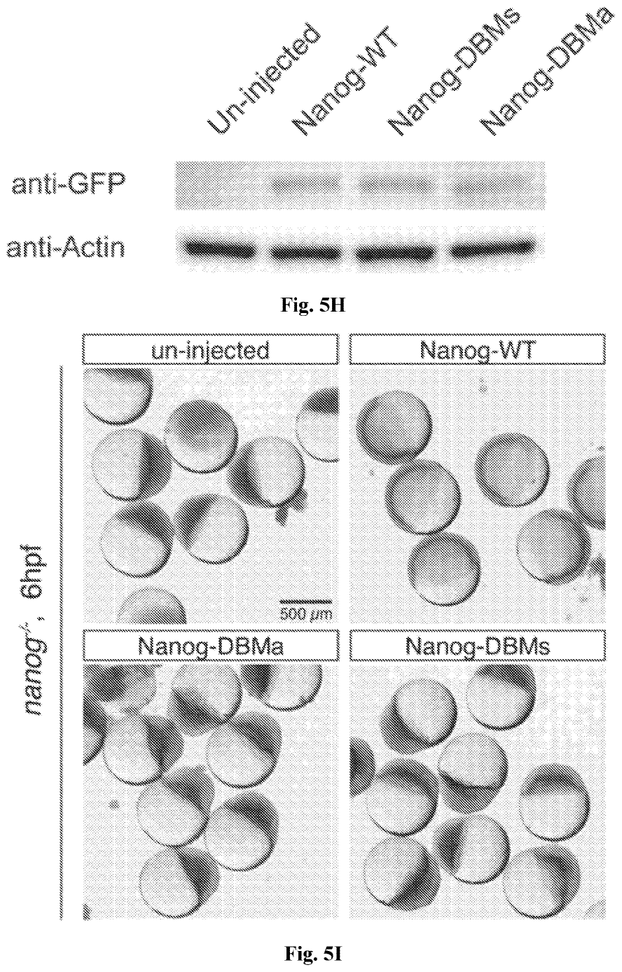

In zebrafish, Nanog functions as a pioneer factor required for genome reprogramming and transcriptional activation after fertilization. To investigate the molecular organization of transcriptional activation during genome activation the present study analyzed the spatial organization of Nanog from the 32-512 cell stage, as transcription is activated. Live imaging of Nanog using a LlamaTag revealed that Nanog forms clusters that are associated with the very early transcription of microRNA-430 (miR-430) (47% of miR-430 transcription sites contain Nanog foci) (FIGS. 1A-1B and 5A-5C). Quantification of Nanog fluorescence revealed that these foci increase the local Nanog concentration 2.5-4 fold (FIG. 5B), consistent with the occurrence of transcription-factor and co-activator foci or hubs observed during embryogenesis and in cultured cells. The present study also observed that Nanog foci were associated with Pol II elongation. Within a cell cycle, the formation of Nanog foci preceded Pol II elongation (FIGS. 1C-1D), as visualized with a genetically encoded mintbody that detects elongating Pol II (Pol II pSer2). After Pol II elongation begins. Nanog foci become less intense, suggesting that Nanog clusters may be evicted or dispersed after initiating transcription. In maternal-zygotic Nanog mutants (MZnanog^{-/-}), Pol II foci are lost and overall levels are reduced at the 256-cell stage, confirming Nanog's role in Pol II recruitment and transcriptional activation (FIGS. 5D-5E). Mutating the Nanog homeodomain to prevent DNA binding abolished Nanog foci, indicating they form in a DNA-binding dependent manner, rather than strictly through protein-protein interactions or aggregation (FIGS. 5F-5I). Thus, Nanog forms DNA-bound hubs that increase its local concentration and are associated with Pol II recruitment and transcriptional activation during genome reprogramming.

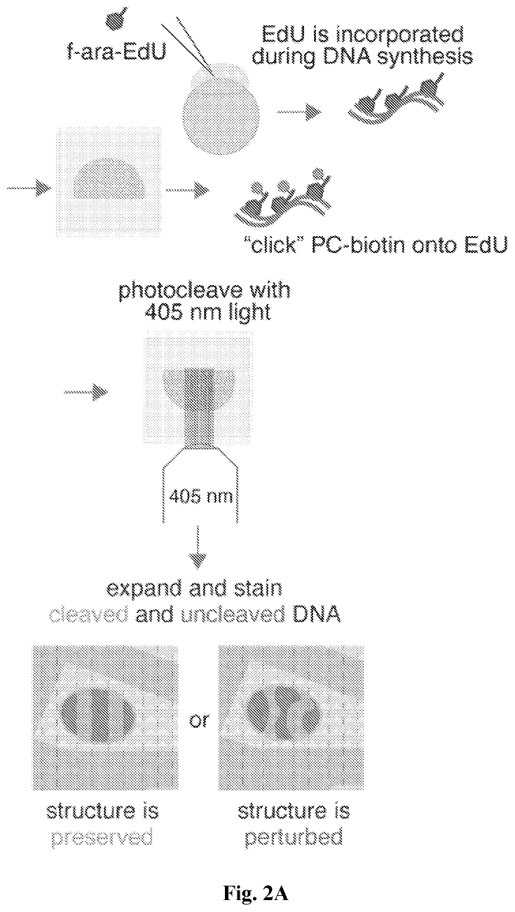

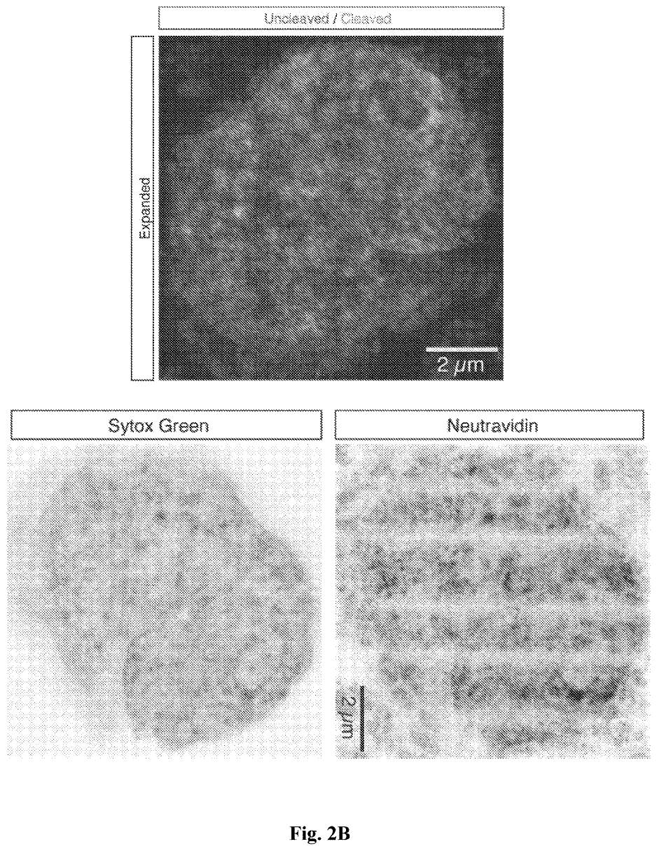

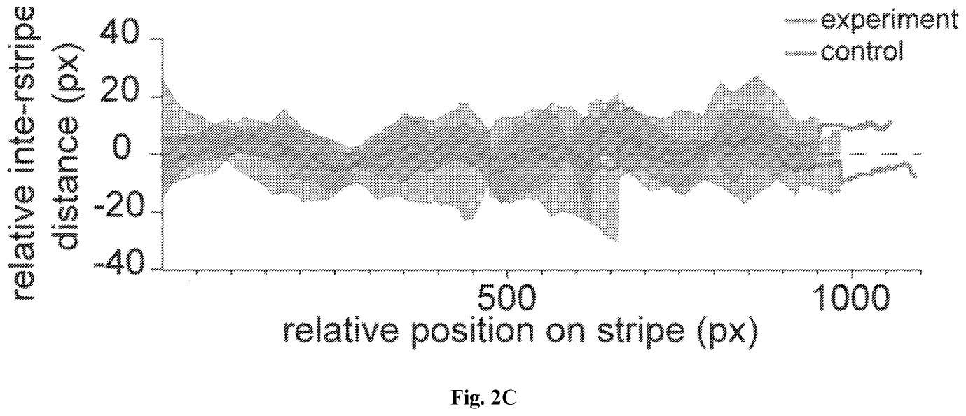

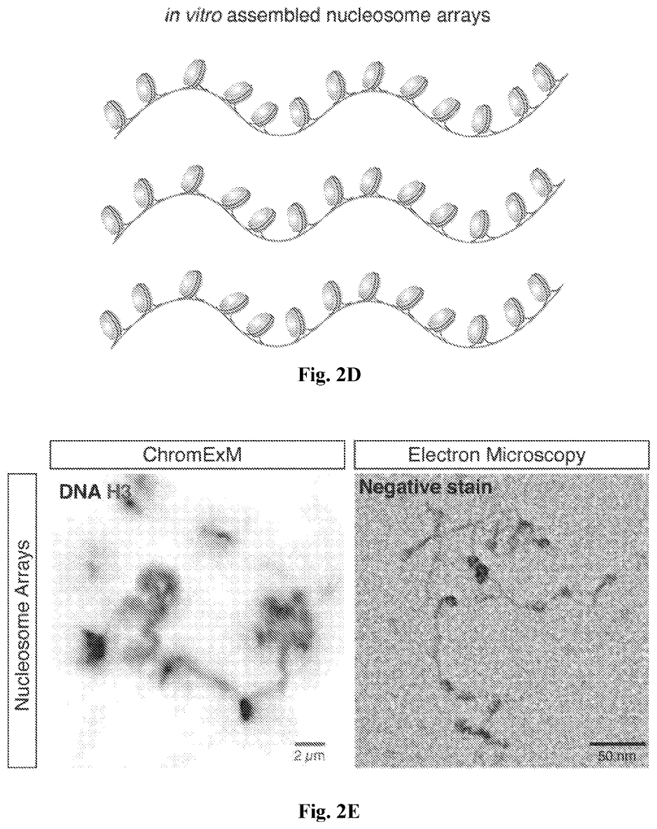

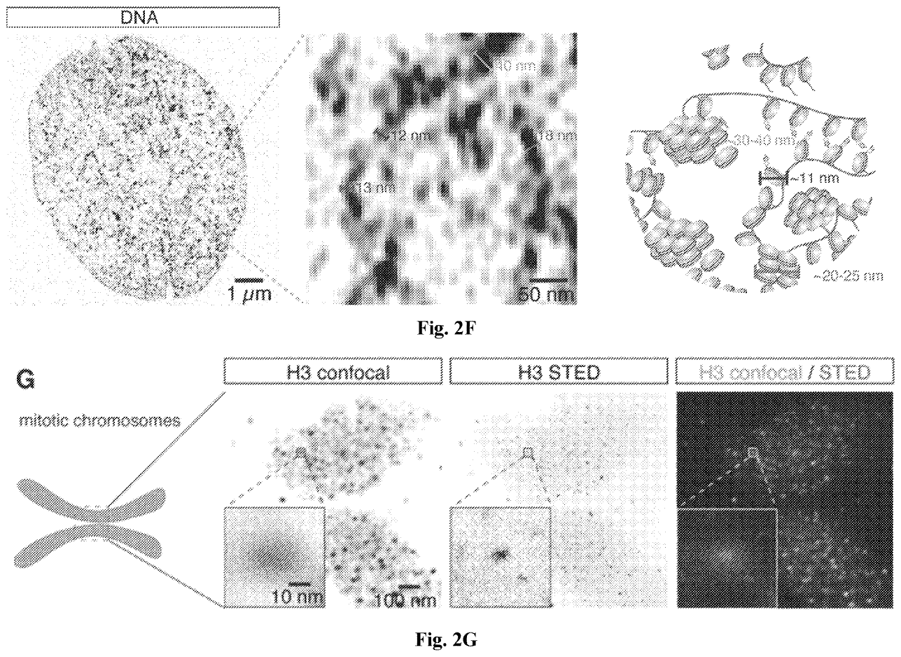

Next, the present study aimed to determine the underlying nanostructure and molecular organization of Nanog and RNA Pol II during chromatin reprogramming and transcriptional activation. To this end, the present study adapted the all-optical super-resolution imaging concept of pan-ExM to include metabolic labeling of DNA and nascent RNA, and proteins to achieve nanometer-resolution visualization of the chromatin, hereafter termed ChromExM (FIGS. 1E-IF). ChromExM involves the direct anchoring of biomolecules to a swellable hydrogel via the addition of acryloyl groups, allowing protein and nucleic acid retention during expansion. The present study achieved an average ~15× linear expansion factor of the nuclei in embryos (FIG. 1G), corresponding to a ~4,000-fold increase in nuclear volume and providing ~15 nm lateral resolution on a confocal microscope and ~3 nm in stimulated emission depletion (STED) super-resolution microscopy (FIGS. 2A-2G).

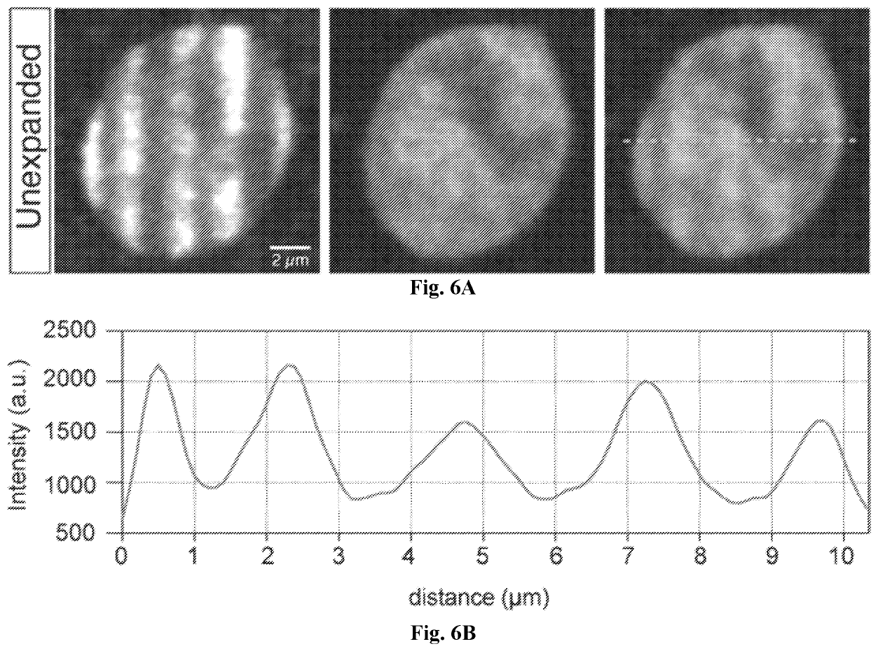

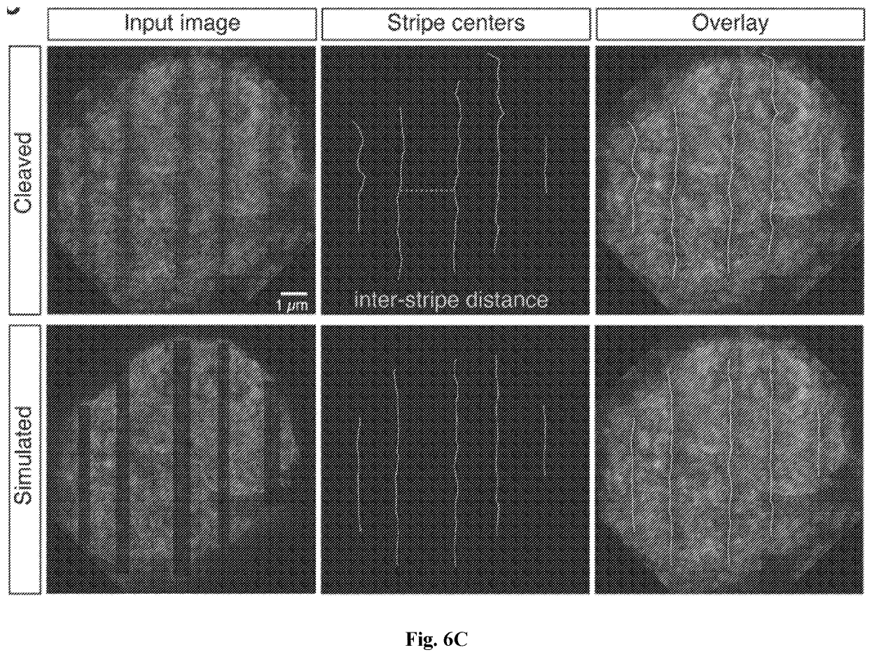

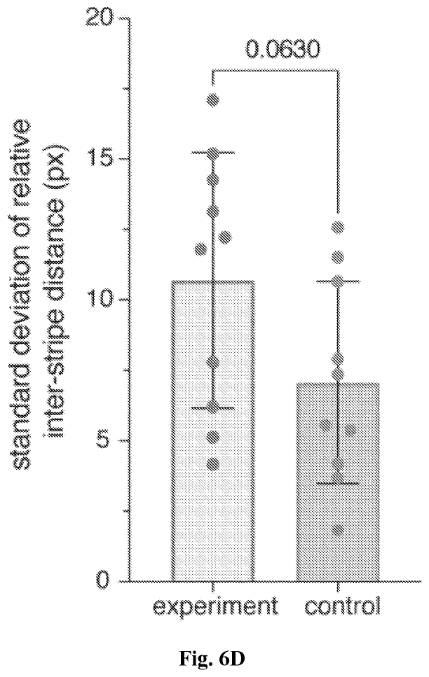

Previous studies have reported conflicting results regarding the isotropy of ~4-8× expansion of chromatin; thus, it is essential assess whether chromatin structure is perturbed by physical expansion. Further, fluorescence intensity decays according to the volumetric expansion factor (~4,000× in ChromExM), imposing a need for highly photostable and bright labelling strategies to detect individual chromatin fibers and nucleosomes. To address these concerns, the present study developed an assay to detect whether chromatin architecture is preserved during ChromExM by photocleaving a pattern of parallel stripes in the DNA before expansion and visualizing the pattern after expansion (FIG. 2A). If chromatin expands isotropically, then the relative position of chromatin chains should be preserved and the photocleaved stripes will remain parallel after expansion. The present study metabolically labelled DNA with f-ara-EdU, and attached a photocleavable biotin azide to EdU. Under patterned UV light, the biotin group is photocleaved and subsequently washed out to generate a striped pattern of biotin-labelled DNA that can be detected with fluorescent Neutravidin (FIGS. 2A, 6A-6B). The present study observed that stripes generated before expansion remain parallel after expansion and show no significant variation in their spacing when compared to simulated perfectly straight stripes generated as controls (FIGS. 2B-2C and 6C-6D). This indicates that chromatin expands isotropically and the relative spatial organization of chromatin is preserved across the nucleus at sub-micron to global length scales by ChromExM (FIGS. 2B-2C).

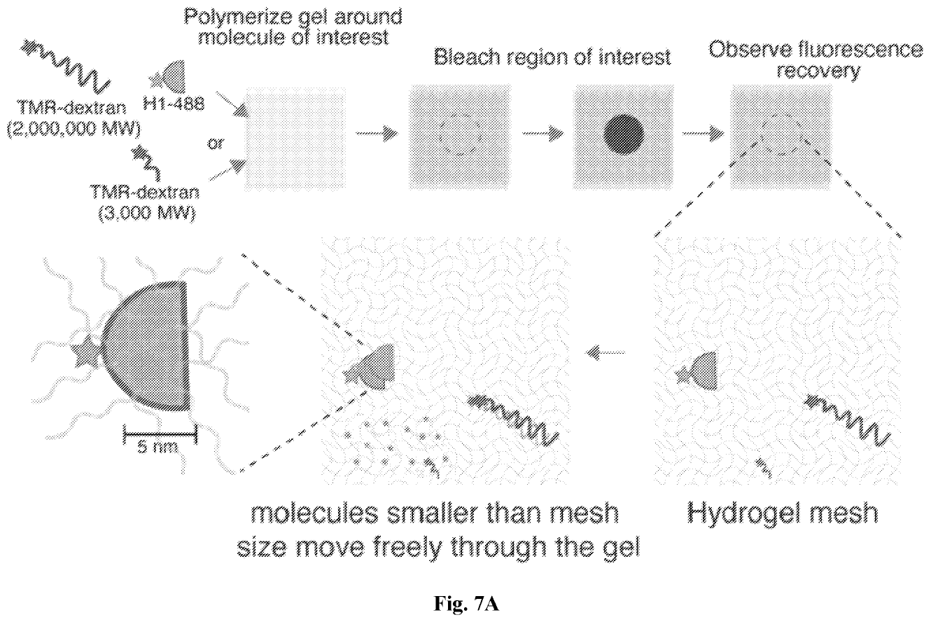

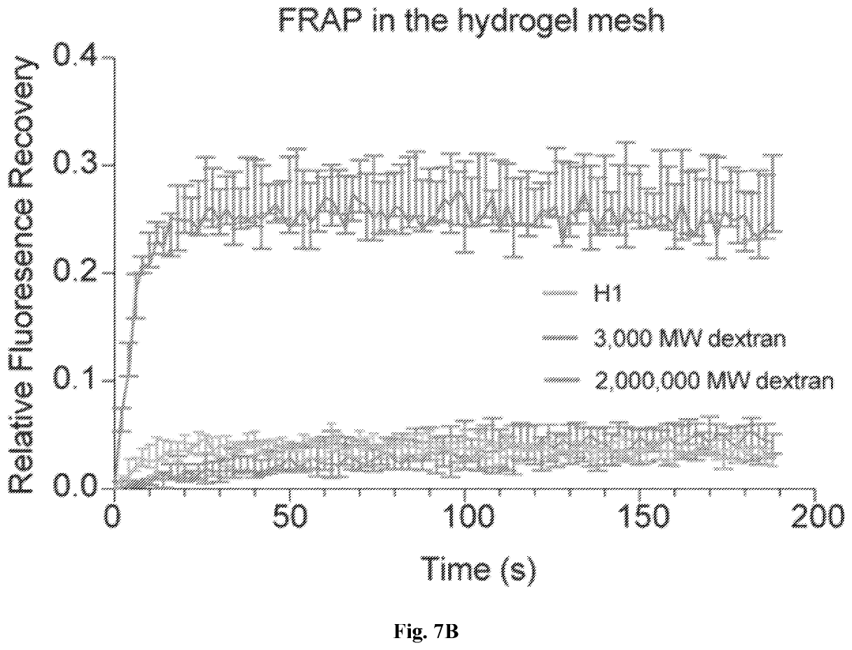

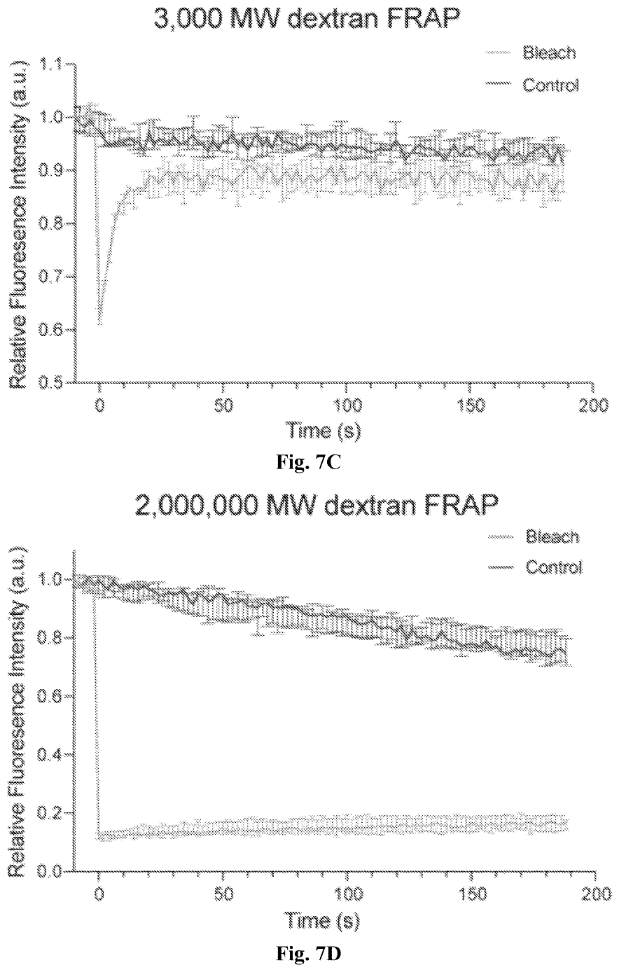

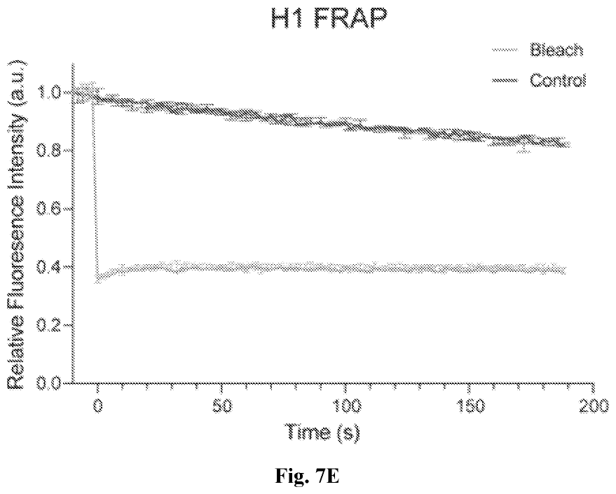

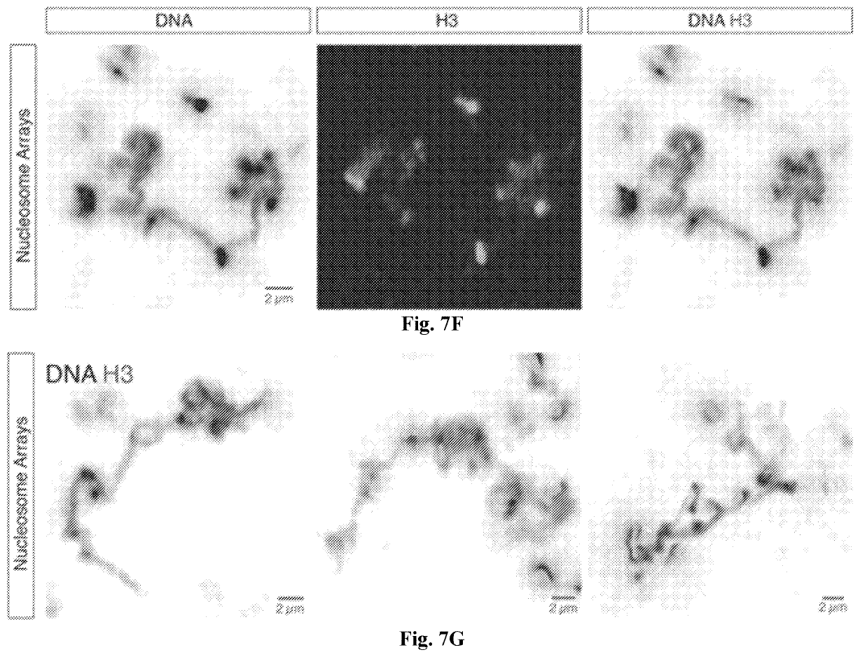

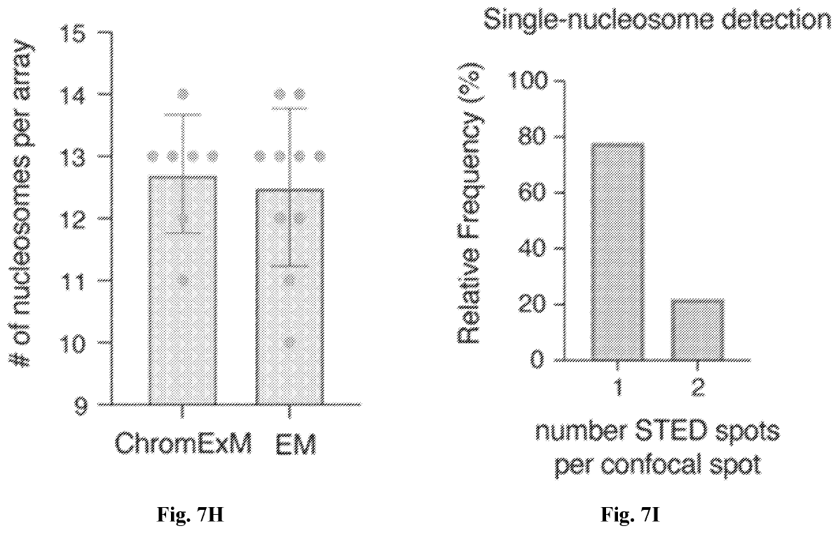

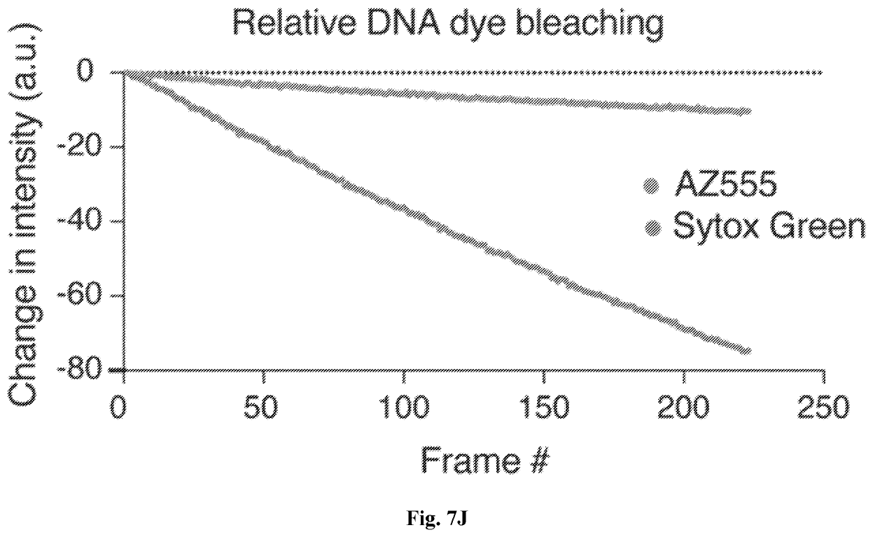

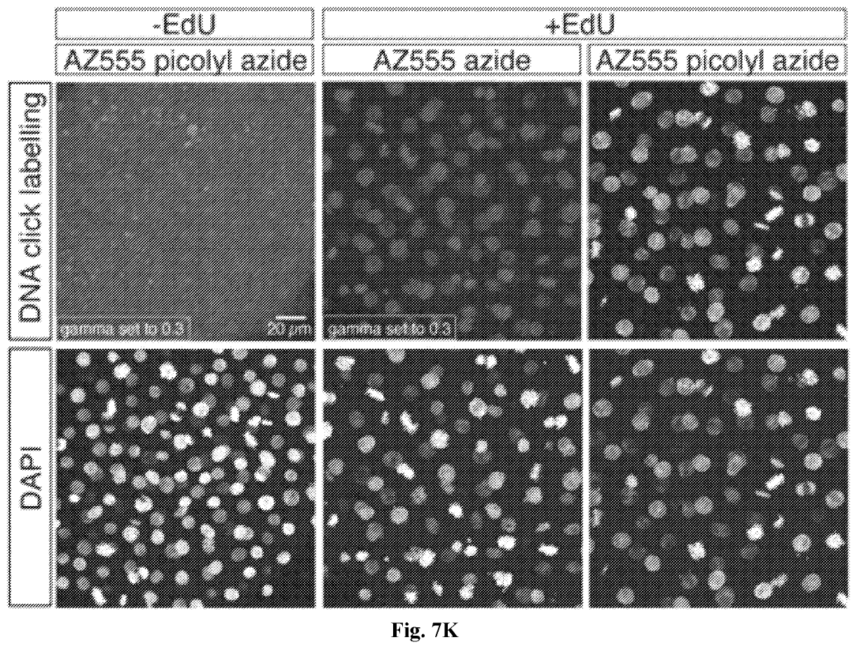

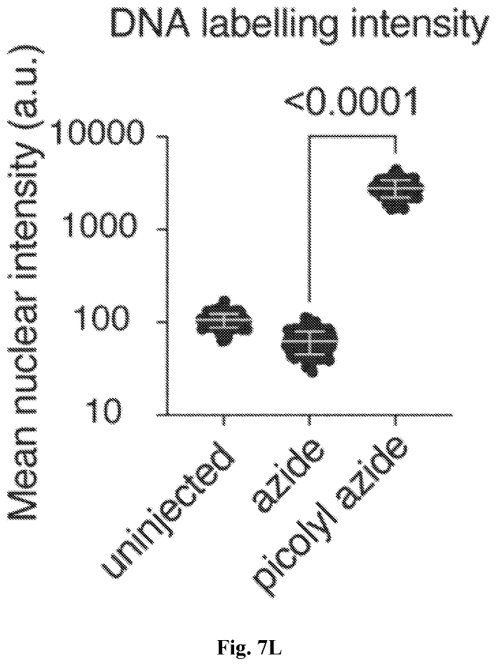

Preservation of chromatin structure at the nucleosomal level depends, in part, on the mesh size of the hydrogel, which impacts the positional uncertainty and anchoring frequency of biomolecules to the gel. To determine whether chromatin structure is maintained at the nucleosome scale, the present study measured the mesh size of the swellable hydrogel herein by assessing the mobility of differently sized molecules in the gel using fluorescence recovery after photobleaching (FRAP) (FIGS. 7A-7E). The present study observed that a 3,000 molecular weight (MW) dextran recovered quickly after photobleaching with a diffusion coefficient of 0.72 μm²/s, but Histone H1 and a 2,000,000 MW dextran did not recover (FIGS. 7A-7E). These results demonstrate that the mesh size is small enough to prevent rapid diffusion of the ~32 kDa H1 protein, suggesting that the gel polymer can anchor the chromatin with subnucleosomal resolution. To functionally test this, the present study compared ChomExM and electron microscopy (EM) of in vitro assembled nucleosome arrays containing 13 nucleosomes along 2.7 kb of DNA (FIGS. 2D-2E and 7F-7G). The present study observed a similar organization of nucleosome arrays in ChromExM and unexpanded EM, with an average of 12.7 and 12.5 nucleosomes detected per array, respectively (FIGS. 2E and 7F-7H). Taken together, these results demonstrate that the local chromatin organization can be maintained at the nucleosomal scale during ChromExM.

To visualize chromatin, the present study metabolically labeled the DNA with f-ara-EdU followed by fluorescent picolyl azide detection after expansion (Uttamapinant et al., 2012, Angewandte Chemie International Edition 51, 5852-5856). This approach was more photostable than the intercalating dye SYTOX Green and increased the labelling intensity ~45-fold compared to standard azides (FIGS. 7J-7L), improving chromatin labeling to overcome the effects molecular decrowding and reduced brightness caused by the ~4,000 fold volumetric expansion. The present study used this approach to resolve chromatin fibers in the nucleus (FIG. 2F) with similar diameters (<12 nm to 40 nm) to those determined by electron microscopy. Nucleosomes are ~10 nm in size, which is at the resolution limit of confocal microscopy after ChromExM. Therefore, the present study performed STED microscopy with ChromExM to improve the resolution by an additional factor of ~five, reaching <3 nm lateral resolution, and demonstrating that ChromExM can resolve individual nucleosomes using H3 immunostaining and confocal microscopy (~80% of nucleosomes detected by confocal are individual nucleosomes by STED) (FIGS. 2G and 7I). The combination of ChromExM with STED provides fast multimodal molecular imaging of the chromatin, approaching <3 nm resolution.

### Example 3: Study of Nanog—Pol II—Chromatin Interactions Using ChromEMT

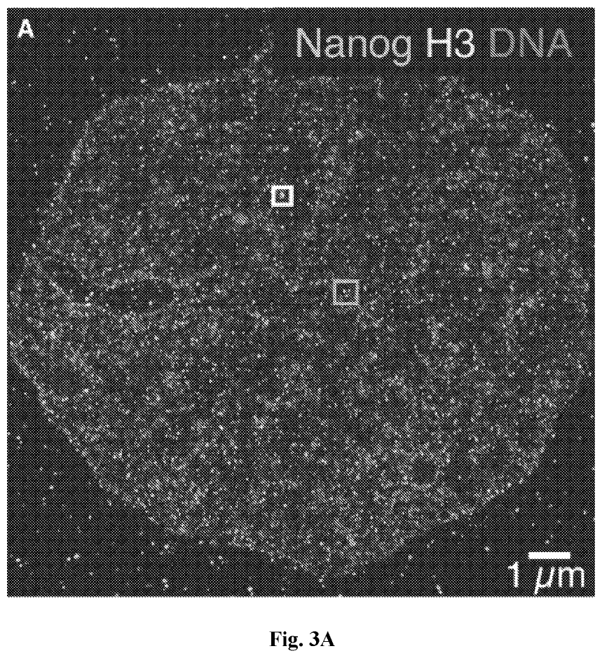

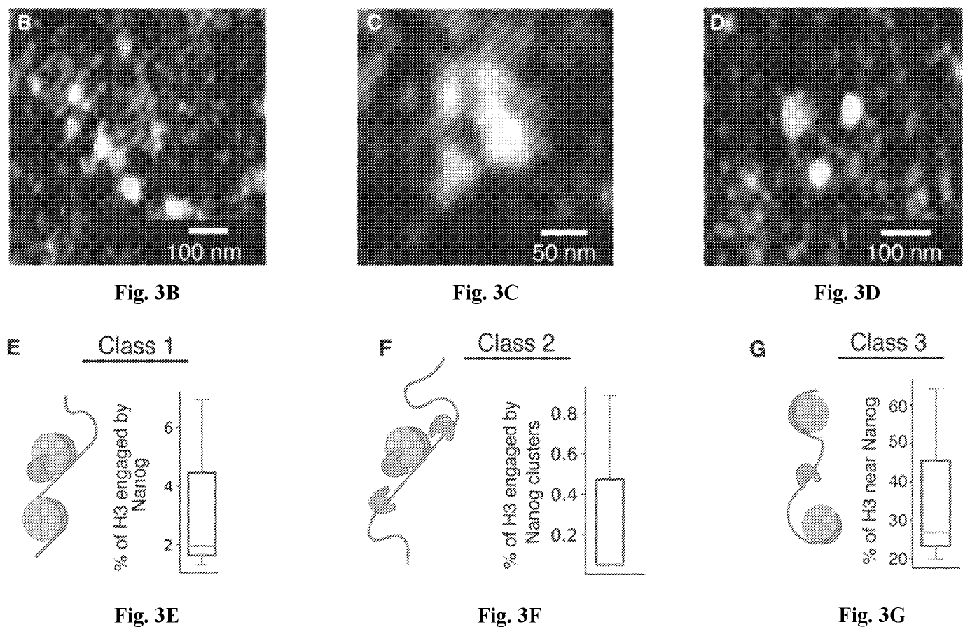

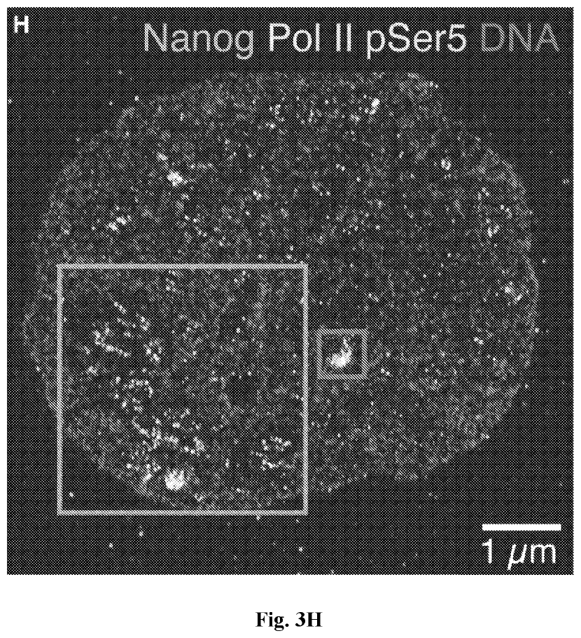

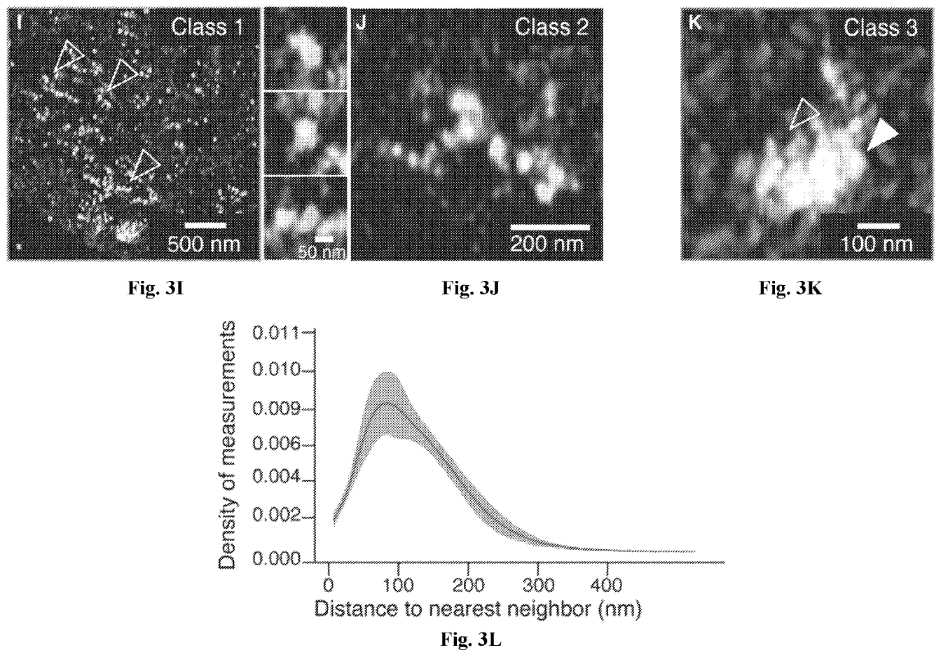

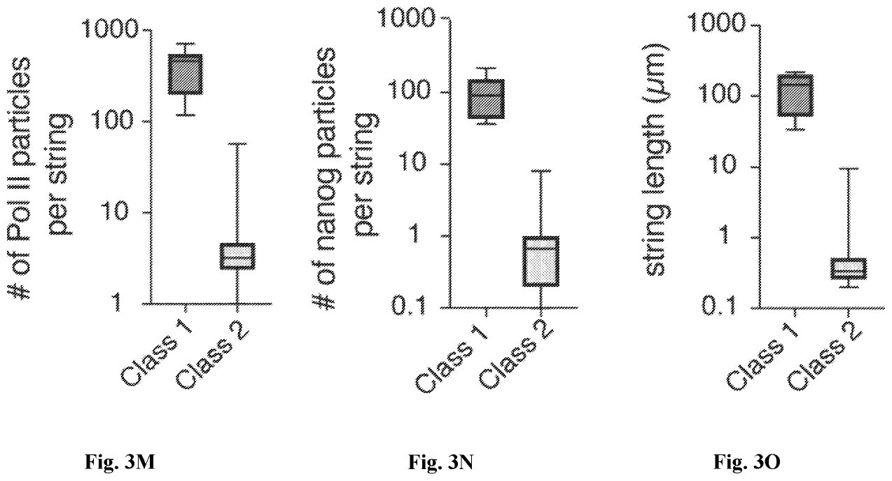

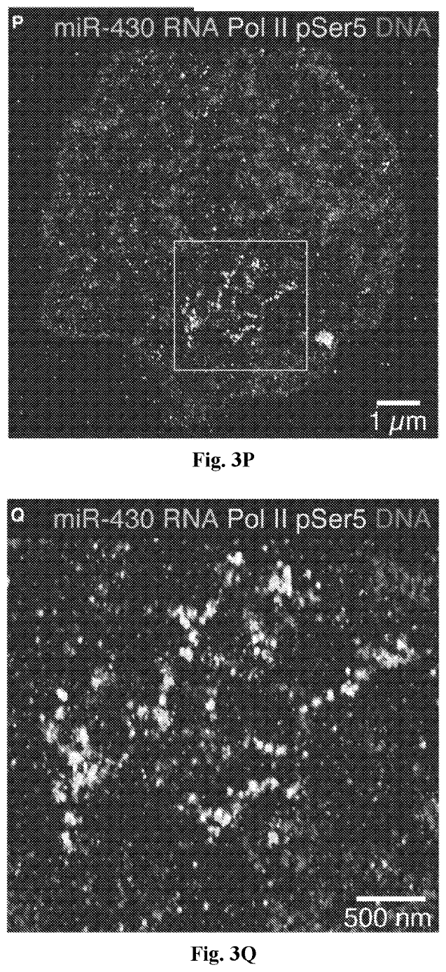

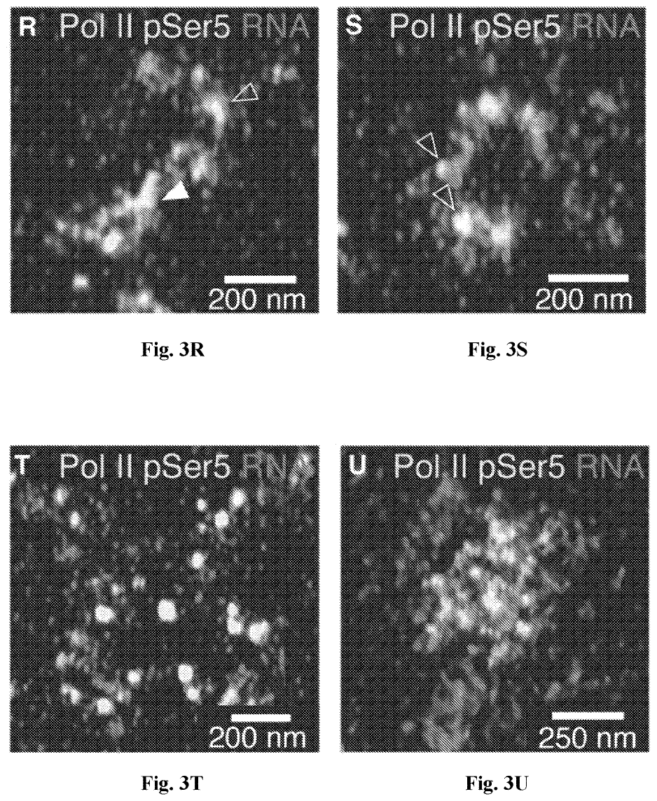

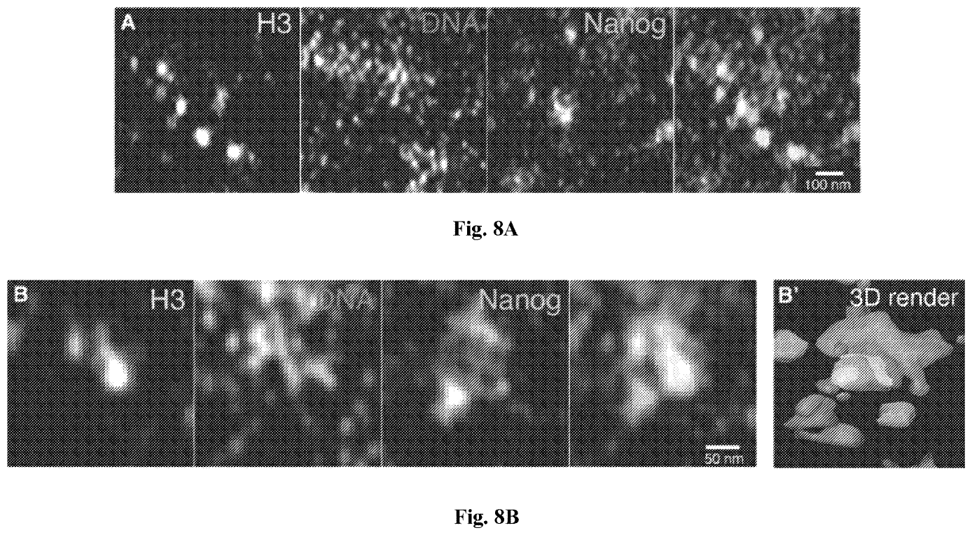

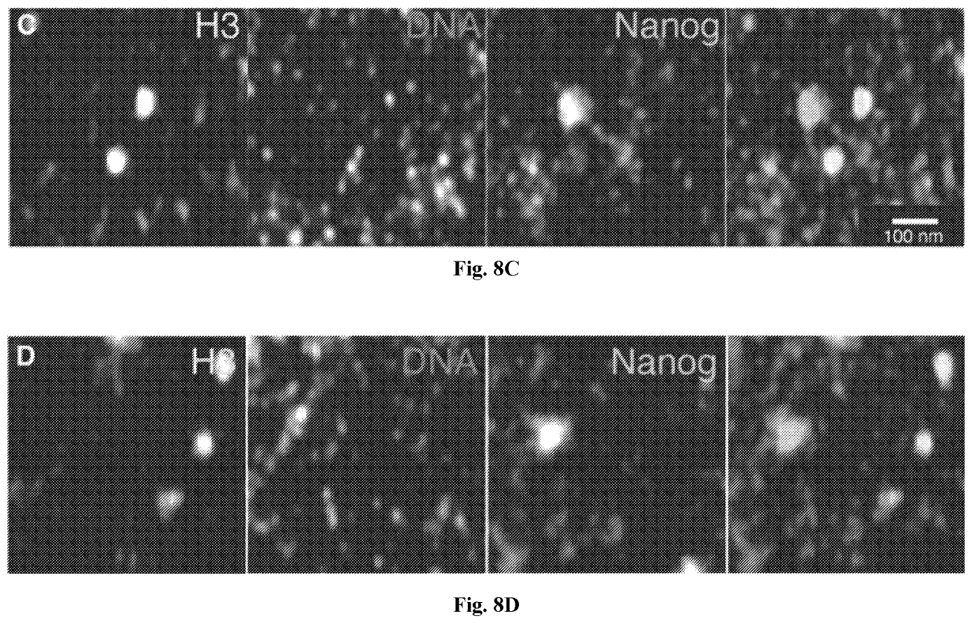

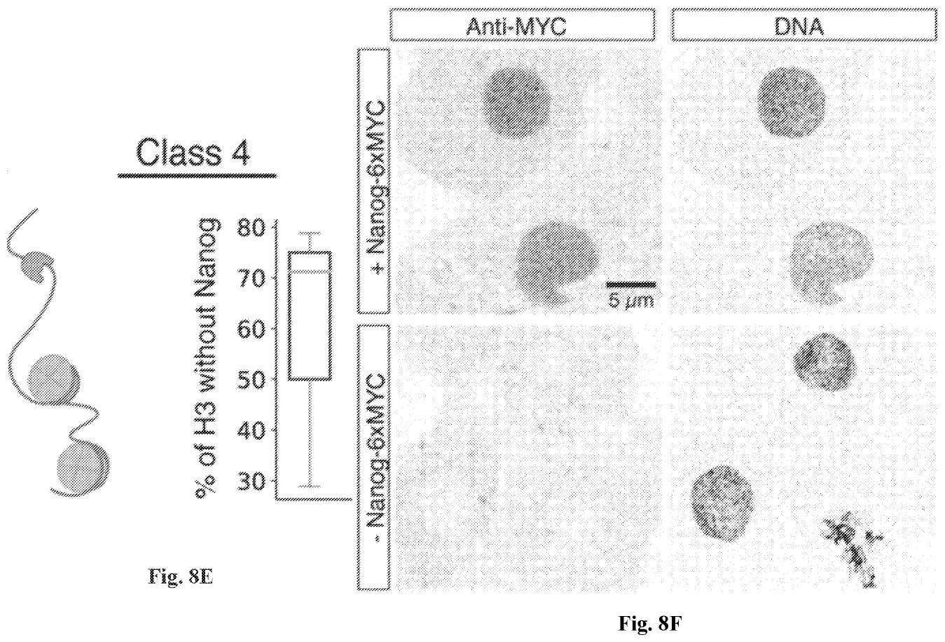

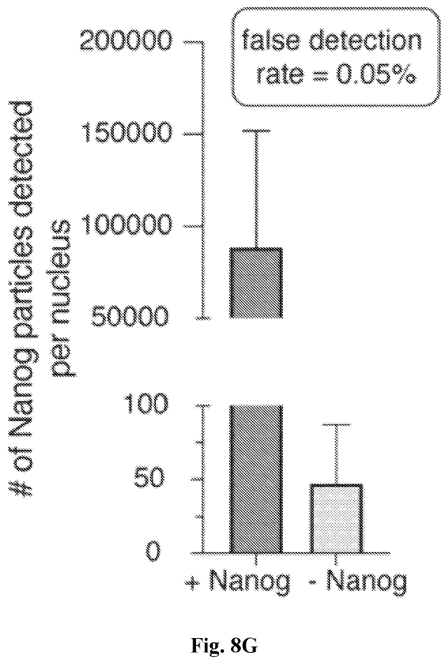

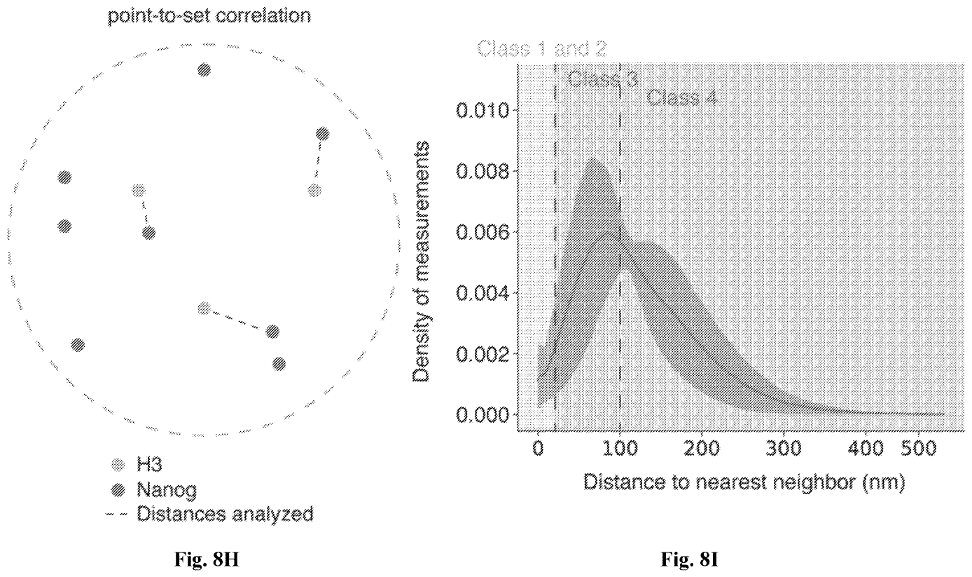

The present study used ChromExM to determine the underlying nanoscale organization of transcription hubs during genome activation by visualizing the molecular scale interactions between the pioneer factor Nanog, nucleosomes, and Pol II at 4 hpf. To analyze how Nanog interacts with chromatin during genome activation, the present study co-stained for Nanog and H3, visualizing an average of 88.619 and 104,347 individual particles of Nanog and H3 per nucleus, respectively, with a false-detection rate of 0.5% for identifying Nanog particles (FIGS. 3A-3D and 8A-8G). Quantitative analysis of the spatial organization of Nanog and H3 particles revealed several classes of Nanog-nucleosome conformations (FIGS. 3B-3G, 8A-8E, and 8H to 8I). In the first class, Nanog was closely associated with nucleosomes (<20 nm distance between Nanog and H3), with 2.0% of H3, and 3.3% of Nanog particles falling into this group (FIGS. 3B, 3E, and 8A), likely indicating a bound state where Nanog is potentially initiating chromatin opening. In the second class, which is a subset of Class 1, Nanog clusters were bound to a nucleosome (>1 Nanog particle within 20 nm and additional Nanog particles within 50 nm), representing recruitment of multiple Nanog particles to the same chromatin regions (0.06% of H3, 0.2% of Nanog) (FIGS. 3C, 3F and 8B). The third class contained Nanog particles distantly associated with nucleosomes (20-100 nm away from each other) (26.9% of H3, 39.7% of Nanog), potentially indicating chromatin regions already opened by Nanog (FIGS. 3D, 3G, and 8C). The fourth class contained nucleosomes and Nanog not closely associated with each other (>100 nm away from each other) (71.2% of H3, 57.0% of Nanog) (FIGS. 8D-8E). The analysis herein identified 88,619 Nanog particles per nucleus, which is consistent with the number of ChIP-seq peaks (~40,000) identified at a similar stage in zebrafish. These measurements are likely the lower bounds of Nanog-nucleosome interactions considering that some nucleosomes may not be detected here. Taken together, the present study was able to characterize pioneer factor-nucleosome organization during chromatin opening.

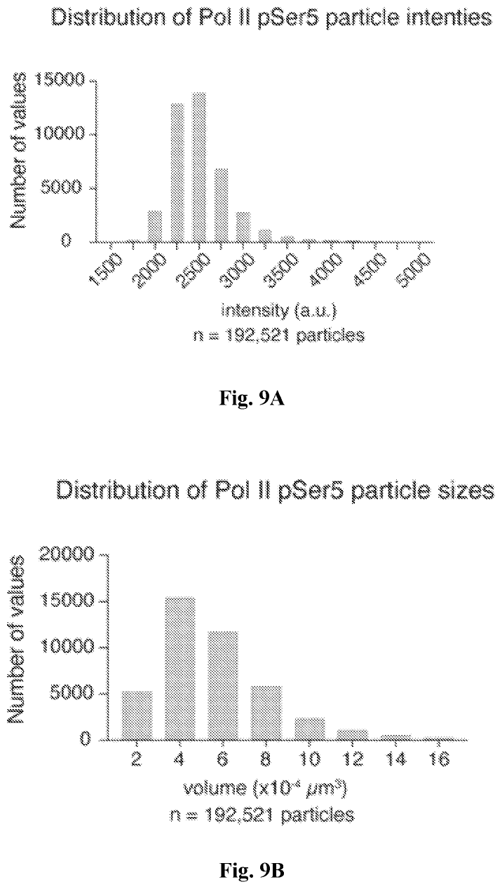

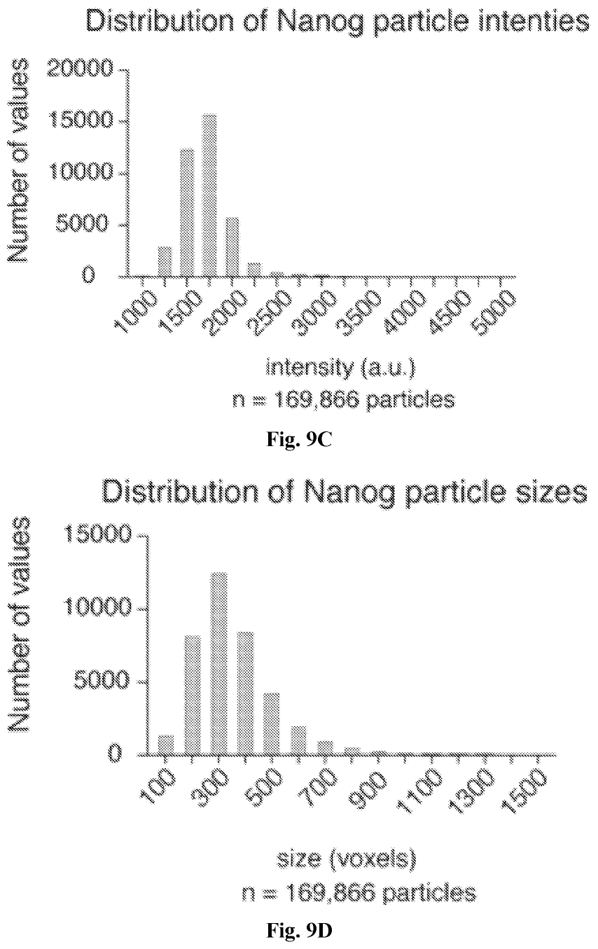

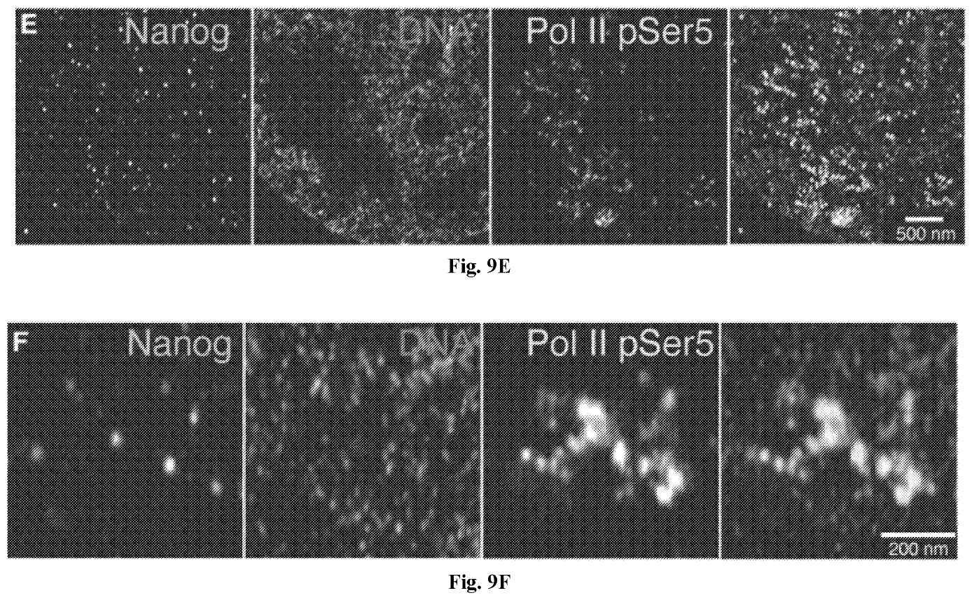

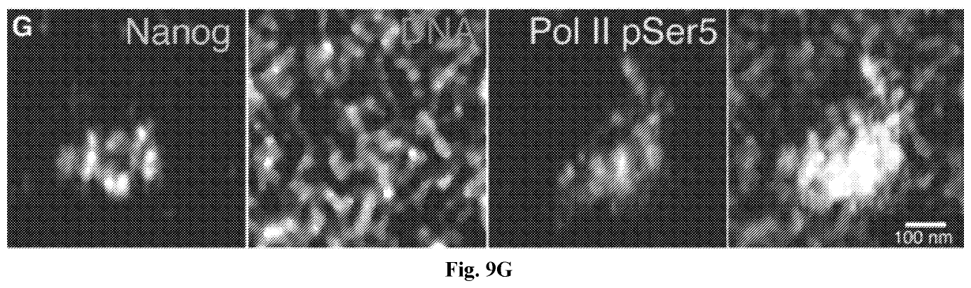

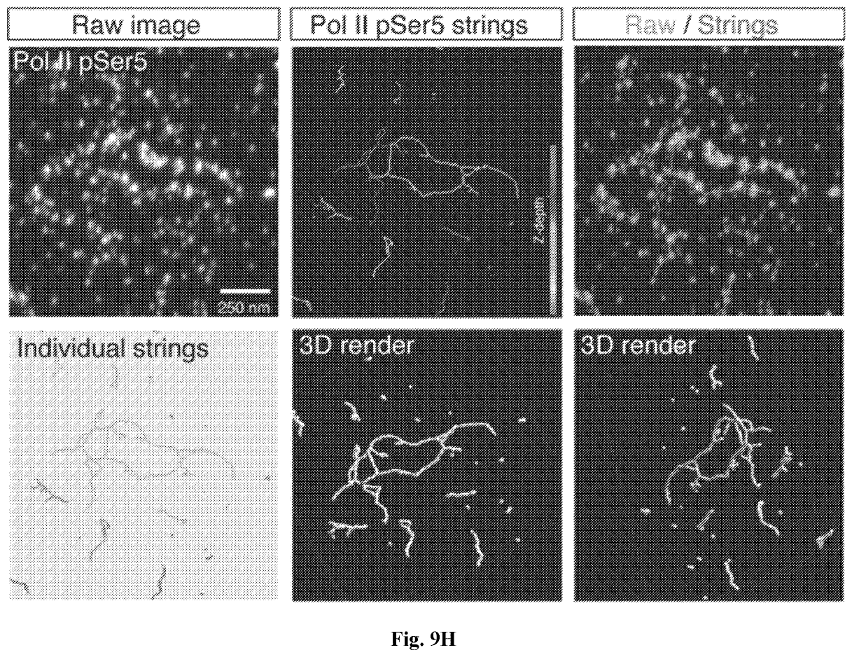

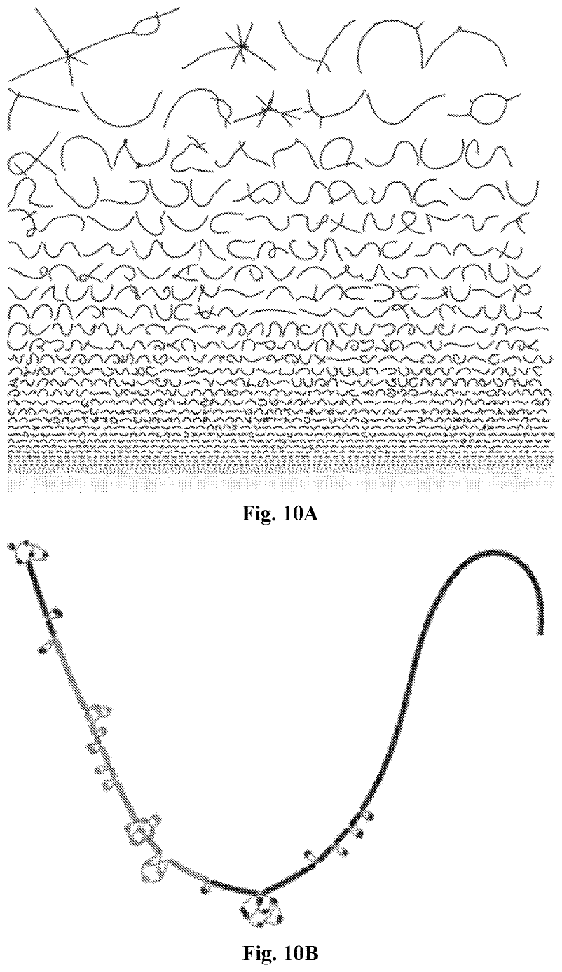

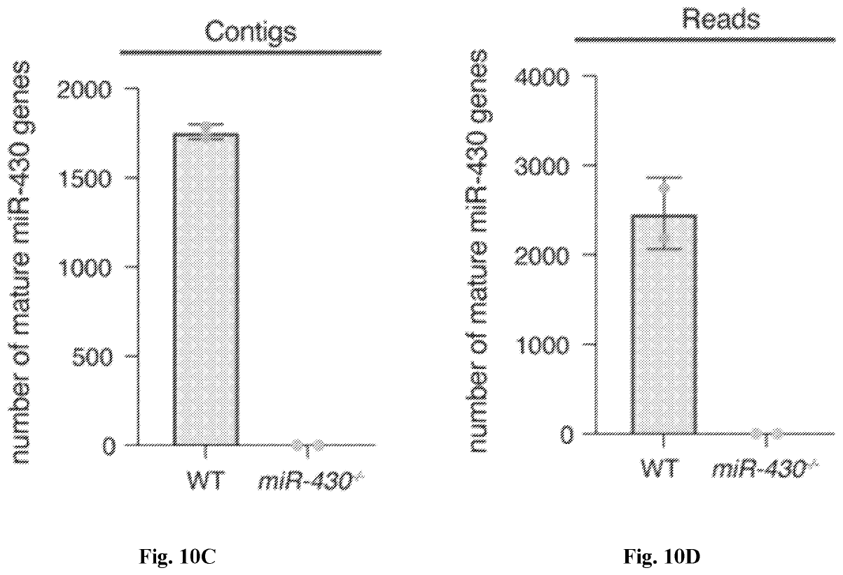

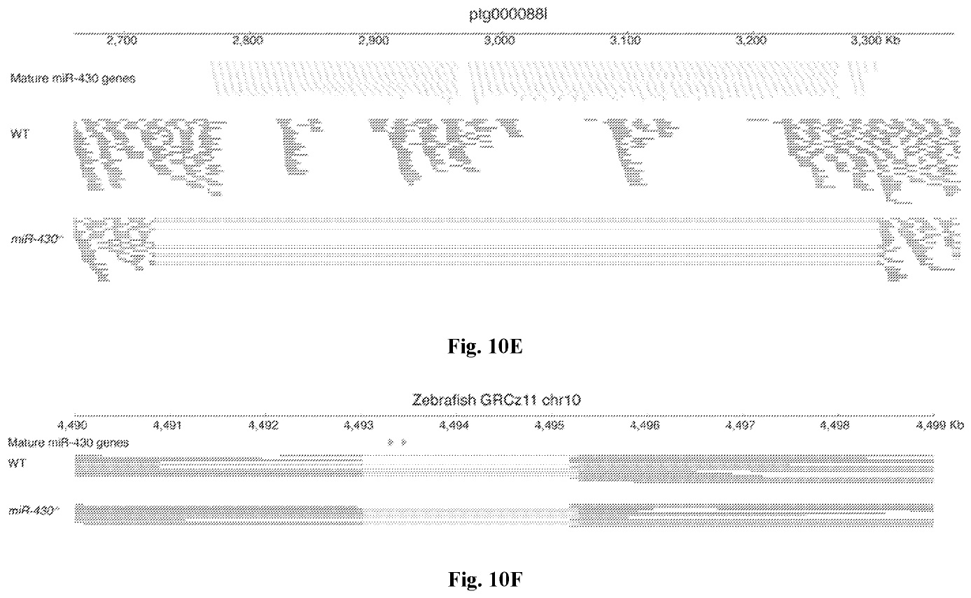

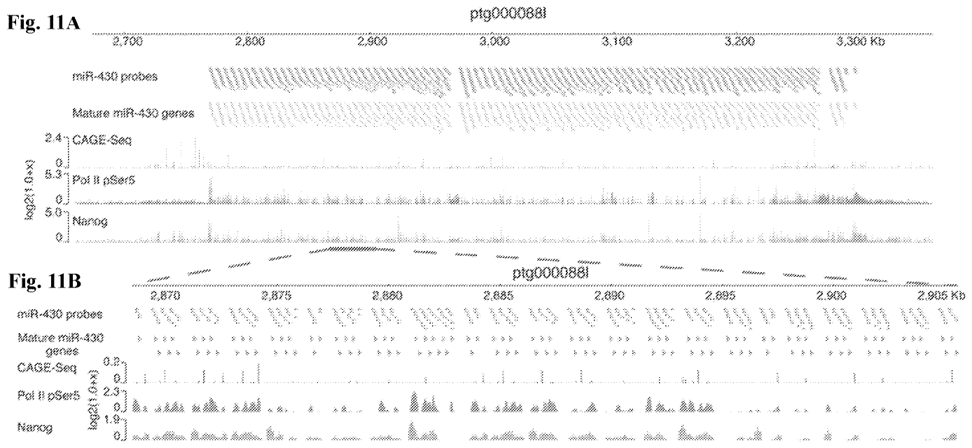

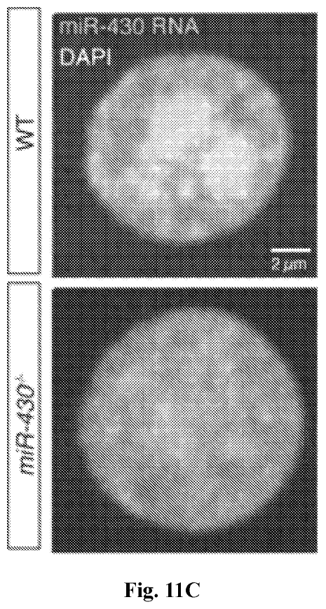

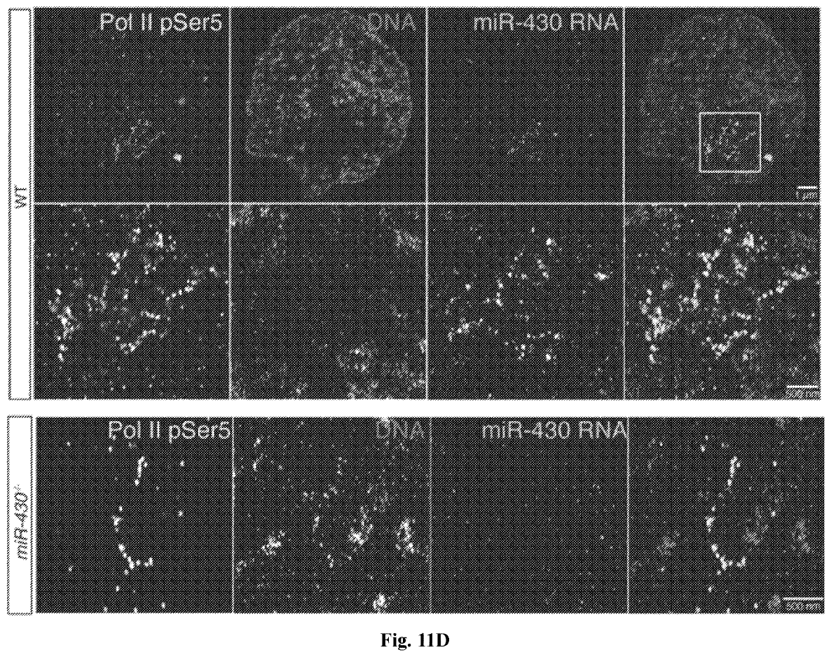

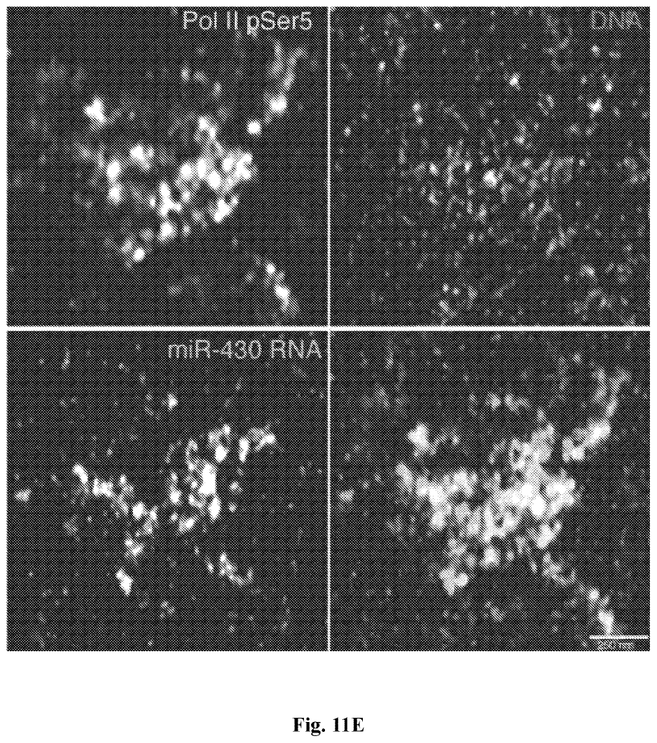

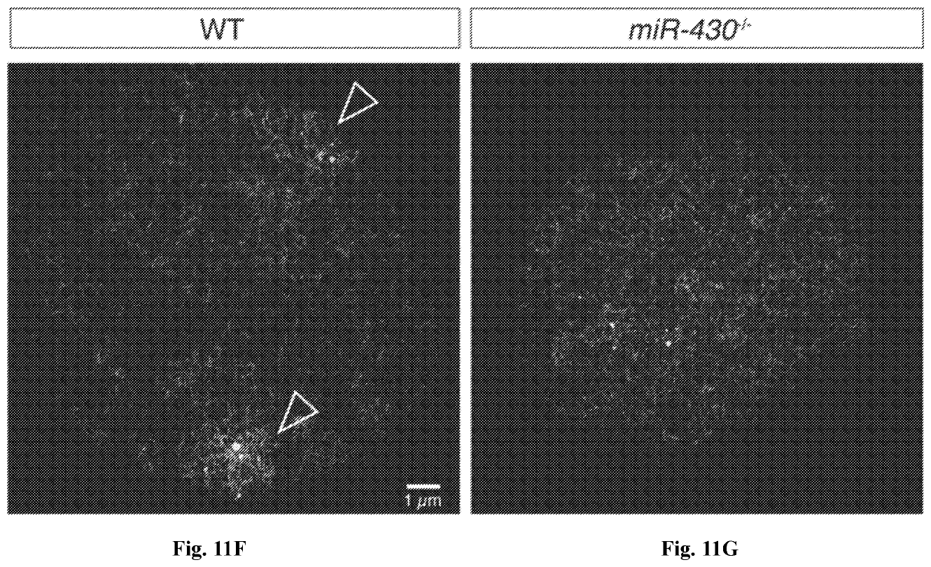

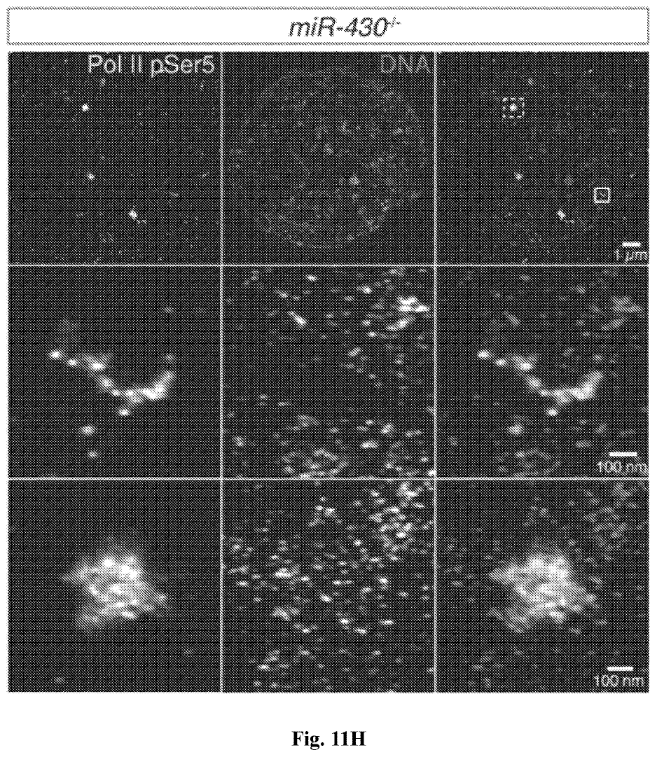

To understand how transcription is organized during chromatin reprogramming, the present study used ChromExM in 4 hpf embryos during the major wave of genome activation. The present study visualized Nanog and Pol II phospho-serine 5 (pSer5), which is deposited on Pol II after recruitment to the promoter (FIGS. 3H-3K and 9E-9G). The present study detected an average of 27,503 Pol II particles per nucleus, which have characteristics consistent with single-molecule detection based on their size and homogenous intensity (FIGS. 9A-9D). Globally, the average distance between Pol II pSer5 and Nanog was 94 nm (FIG. 3L), with 19% of Pol II pSer5 particles being within 50 nm of Nanog particles, consistent with Nanog's role in Pol II recruitment. However, the present study observed that Pol II exhibited three distinct types of organization (FIGS. 3I-3K and 9E-9H). The first class involved large groupings of interspersed Pol II pSer5 and Nanog particles (412 Pol II pSer5 particles and 100 Nanog particles on average, 132 μm mean length) arranged like beads on a string which is referred to as Class 1 strings herein and occurred twice per nucleus in 86% of nuclei observed (FIGS. 3I, 3M-3O, and 9E). RNA fluorescence in situ hybridization (RNA FISH) for miR-430 revealed that Class 1 strings represent sites of active miR-430 transcription (FIGS. 3P, 3Q, and 11A-11E). To validate this result, the present study assembled the highly repetitive miR-430 locus using single haploid embryo long-read genome sequencing and identified a single, continuous ~550 kb miR-430 locus (FIGS. 10A-10F), corresponding to an estimated length of ~200 μm which is consistent with the 132 μm length of Class 1 strings (FIG. 3O). This locus has been estimated to have >300 promoters capable of transcribing primary miR-430 transcripts, consistent with the ~1,800 mature miR-430 genes detected in the assembly (FIGS. 10C-10D). Similarly, the present study detected ~400 Pol II pSer5 particles in Class 1 strings, which likely represent binding at these promoters (FIG. 3M). The string-like structure connecting Pol II and Nanog particles is consistent with Pol 11 and Nanog binding profiles observed by ChIP-Seq at this locus (FIGS. 11A-11B). These Class 1 strings and miR-430 RNA FISH signal are lost in miR-430^{-/-} embryos (FIGS. 11C, 11D, 11F, and 11G), which the present study confirmed lack all miR-430 genes using long-read genome sequencing (FIGS. 10C-10F). Together, these results demonstrate that Class 1 strings show the nanoscale organization of the transcriptionally active miR-430 cluster.

The second class of Pol II pSer5 organization involved multiple Pol II particles (2 to 70; mean of 4 particles per string) organized into smaller-scale strings, with an average length of 831 nm (FIGS. 3J, 3M-3O, 9F, and 9H). In this class, 37% of Pol II pSer5 particles were within 50 nm of Nanog particles, consistent with Nanog's role in Pol II recruitment. The present study hypothesized that these linear Class 2 Pol II pSer5 strings represent individual genes loaded with Pol II arranged in single-file lines. Class 2 strings are still present in miR-430^{-/-} embryos (FIGS. 11D, 11G, and 11H) indicating that they represent other transcribed genes regulated by Nanog and other transcription factors during genome activation. This processive organization of Pol II is reminiscent of active transcriptional elongation. In other cases, the present study observed branched Pol II pSer5 strings (FIGS. 3J, 9F and 9H), consistent with the transcription factory model where multiple genes are in close proximity sharing a regulatory Pol II hub. Indeed, the present study detected an average of 822 Pol II pSer5 strings per nucleus, consistent with the ~1,700 active zygotic genes at 4 hpf.

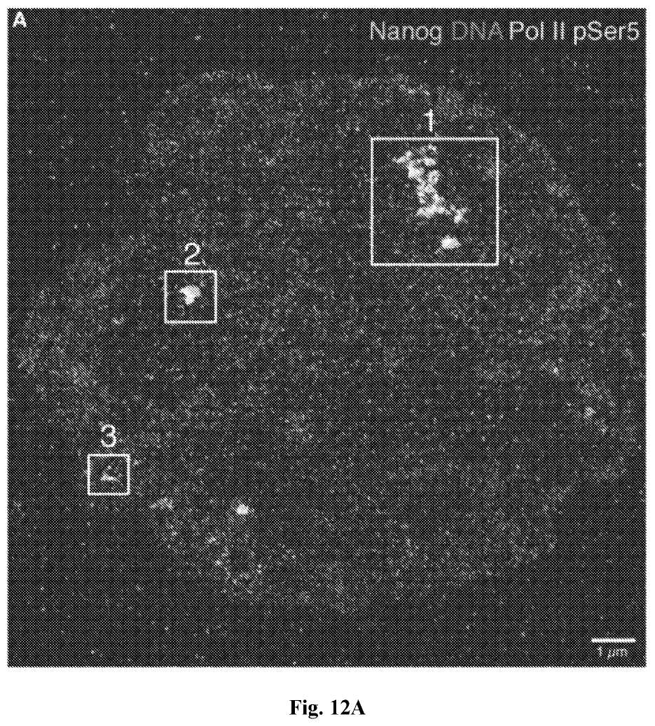

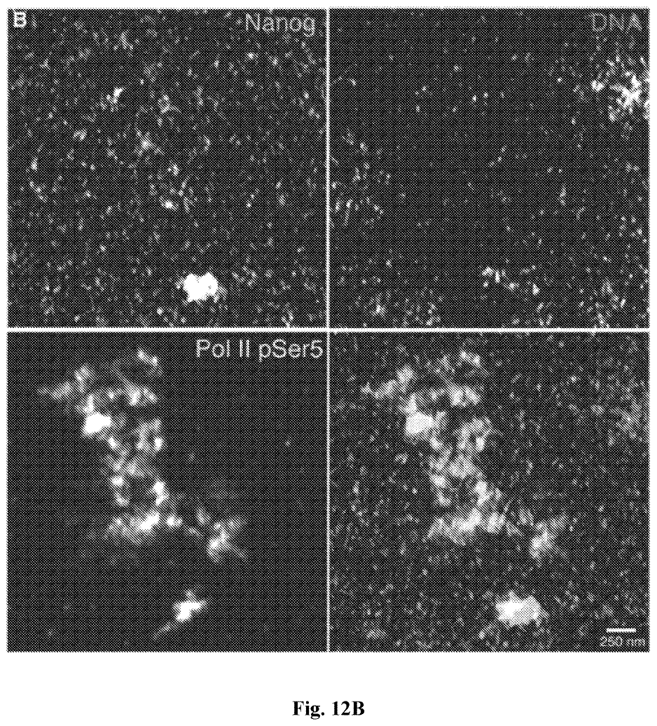

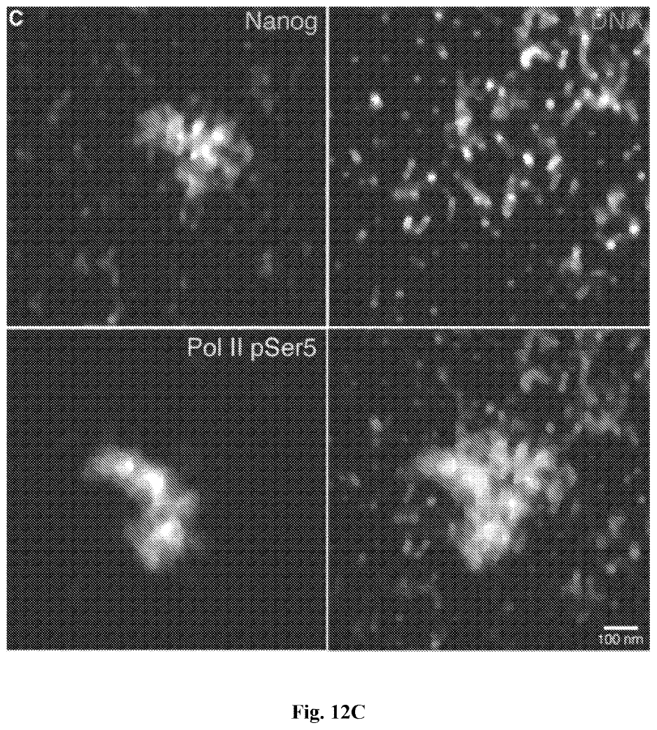

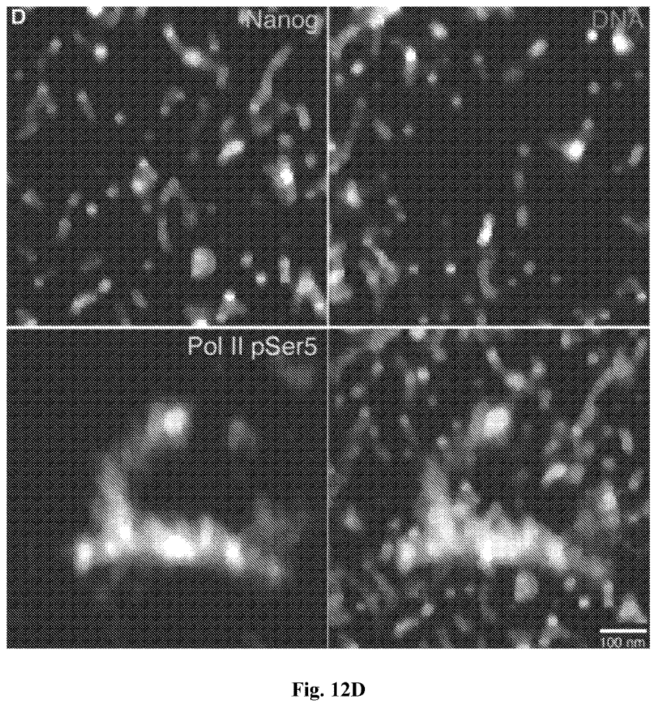

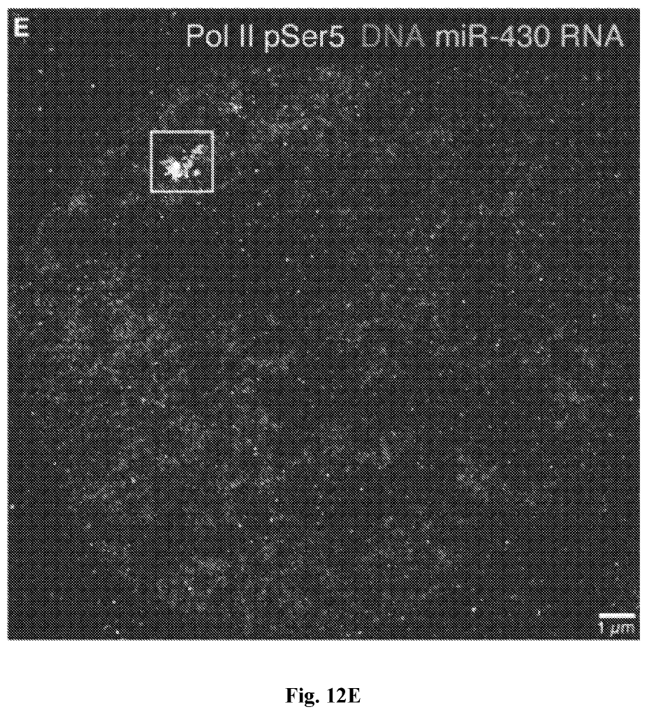

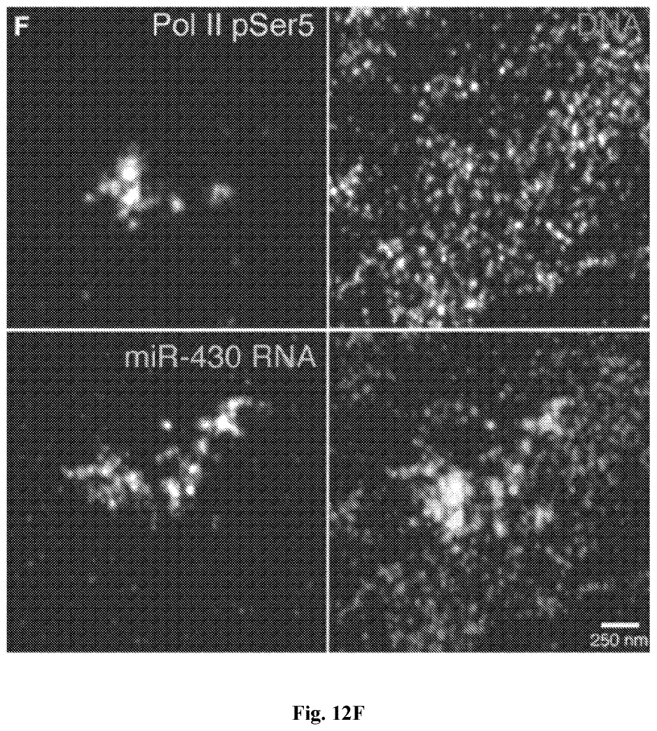

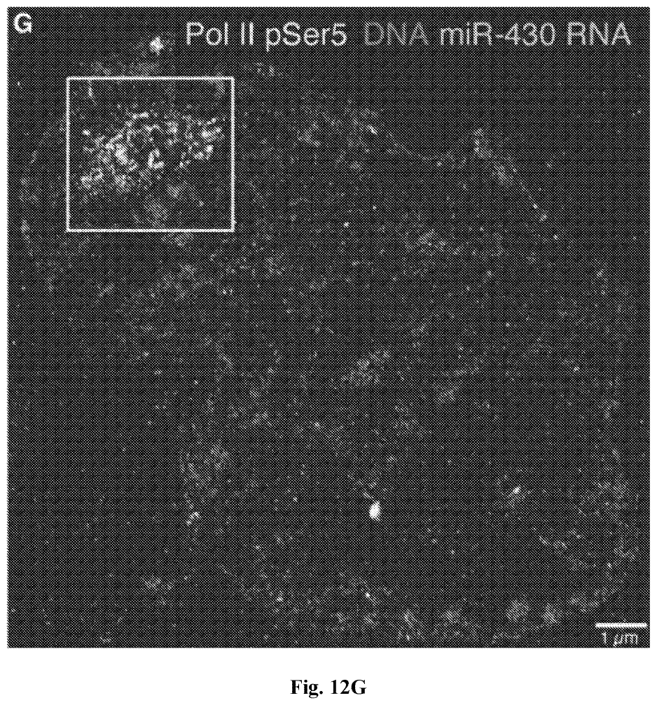

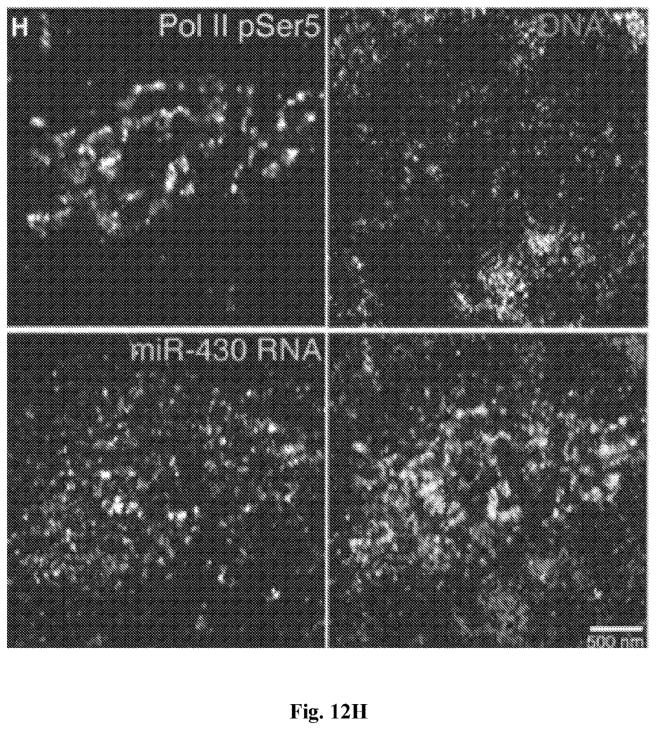

The third class of Pol II organization involved ‘macroclusters,’ (FIGS. 3K and 9G) similar to those previously observed by super-resolution imaging. The present study detected an average of 28 Pol II pSer5 macroclusters per nucleus. In cases where these macroclusters were occupied by both Nanog and Pol II, the present study was able to resolve distinct regions within the cluster occupied solely by Nanog and Pol II (FIGS. 3K and 9G), as if the two factors were tethered on distinct DNA elements. These examples are consistent with Nanog bound to enhancers residing in close proximity (<50 nm) to Pol II bound at the promoter, consistent with Pol II recruitment at these sites by Nanog. Similarly, the present study observed all three classes of Pol II pSer5 organization during the minor wave of genome activation at 2.5 hpf, although there were fewer active sites, consistent with lower levels of transcription at this time (FIGS. 12A-12H). This suggests that each class of Pol II organization represent a generalizable state of Pol II organization broadly used throughout genome activation. At 4 hpf, the present study observed that there are an average of 5.3 Pol II particles within 200 nm of each Nanog particle among all three classes, suggesting that multiple Pol IIs are recruited by Nanog. Among the macroclusters, the present study also observed multiple cases where a string of RNA Pol II emanated from the cluster (FIG. 3K), raising the question of how these two distinct Pol II pSer5 structures may function.

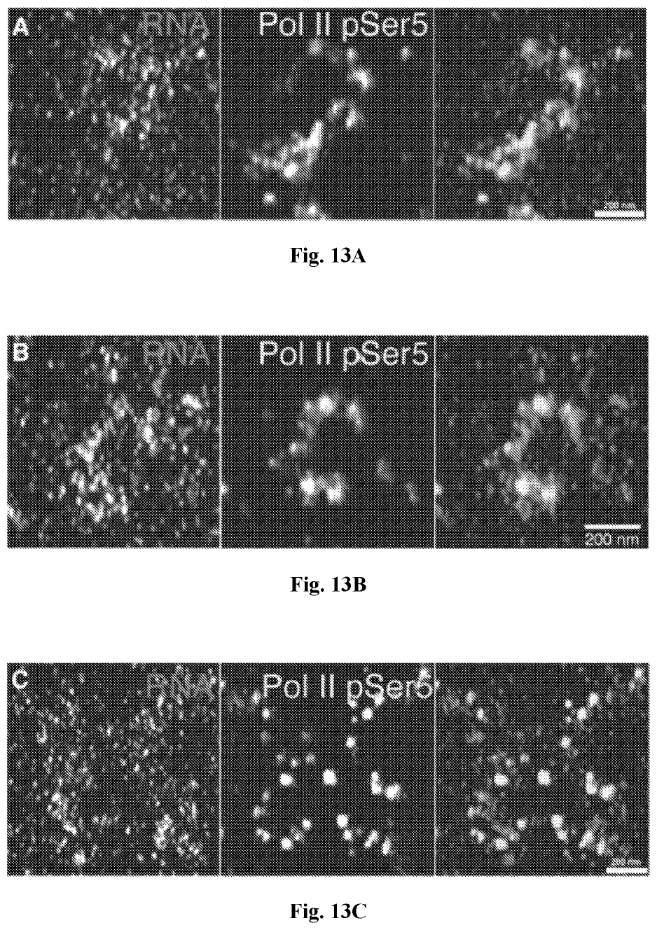

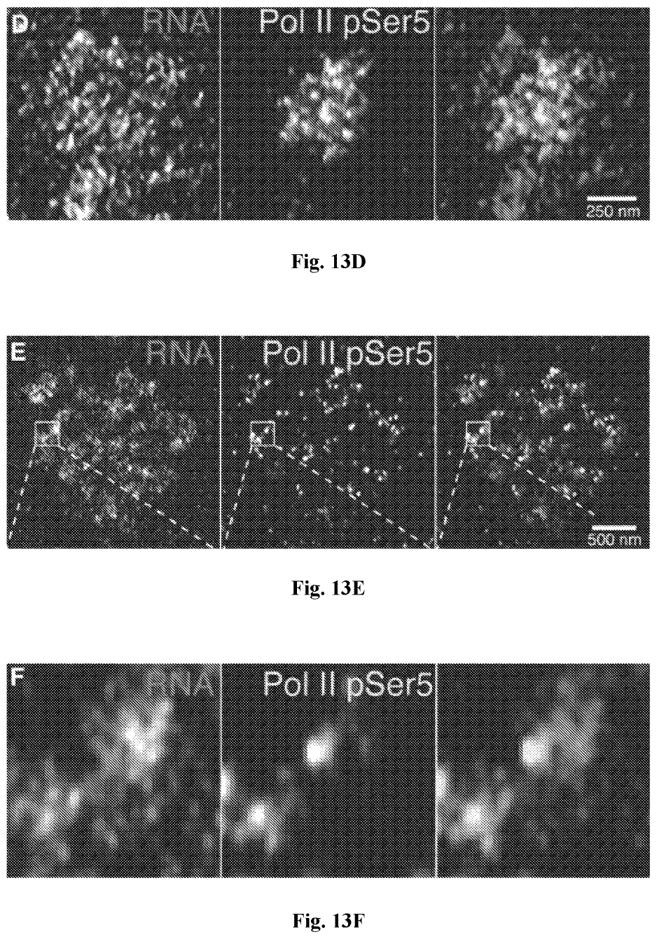

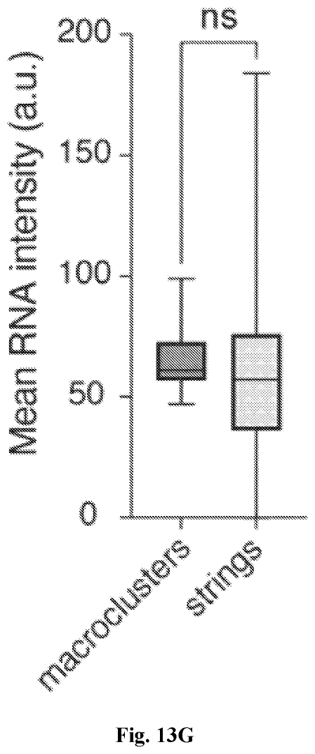

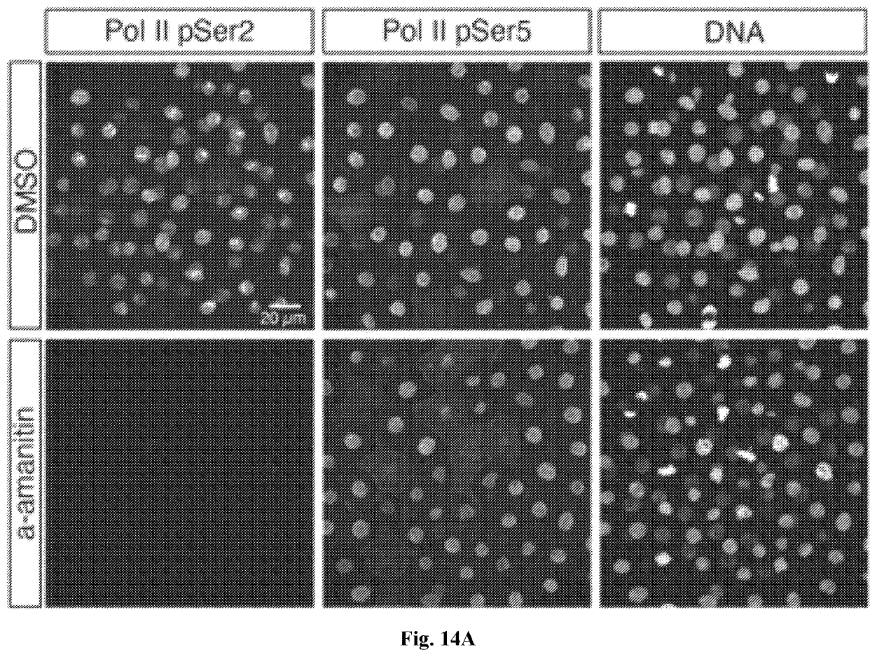

The present study hypothesized that both large and small Pol II pSer5 string structures represent actively transcribing Pol II, which has exited the shared regulatory hub where Nanog, Pol II, and potentially other co-activators congregate to control gene expression. To test this idea, the present study combined metabolic RNA labelling using 5-ethynyl uridine (EU) with ChomExM labelling for Pol II pSer5, and observed nascent transcripts associated with all three classes of Pol II pSer5 structures (FIGS. 3R-3U and 13A-13F). The present study observed a wide range of transcription levels associated with the Pol II pSer5 strings, suggesting that they may encompass actively transcribed gene bodies as well as paused promoters (FIG. 13G). The organization of these transcriptionally active Pol II pSer5 strings is consistent with Pol II elongation along the gene body of individual genes, where the present study visualized multiple Pol II pSer5 particles extruding nascent transcripts in concert (FIGS. 3R-3T, 13A-13C, and 13E-13F). Taken together, these results support a model where Nanog and Pol II clusters are in close physical proximity and form a regulatory hub that functions to activate transcription as Pol II exits the hub, forming a string along the gene body (FIGS. 3R and 13A).

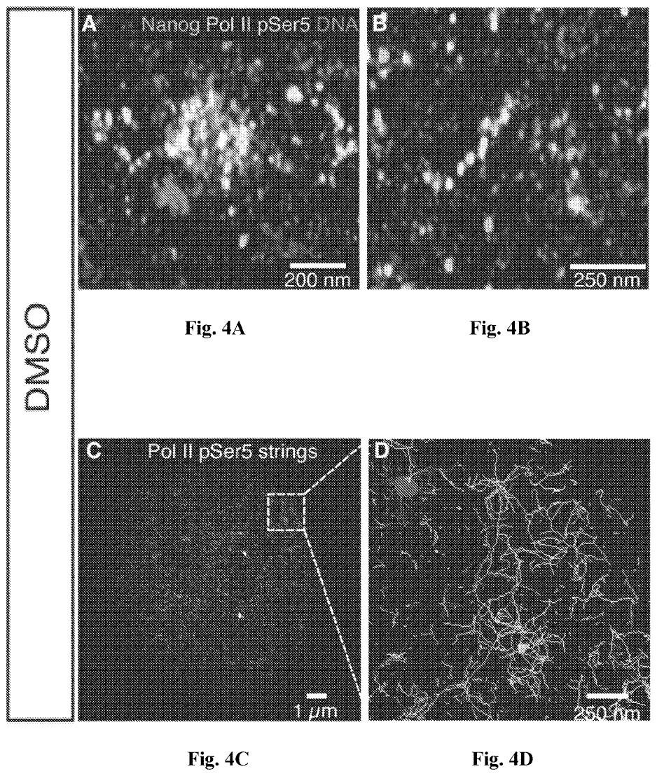

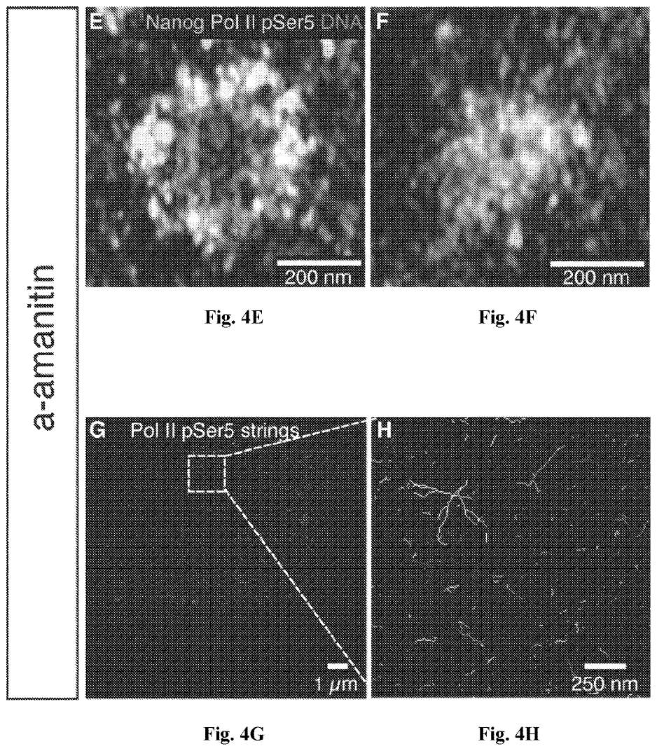

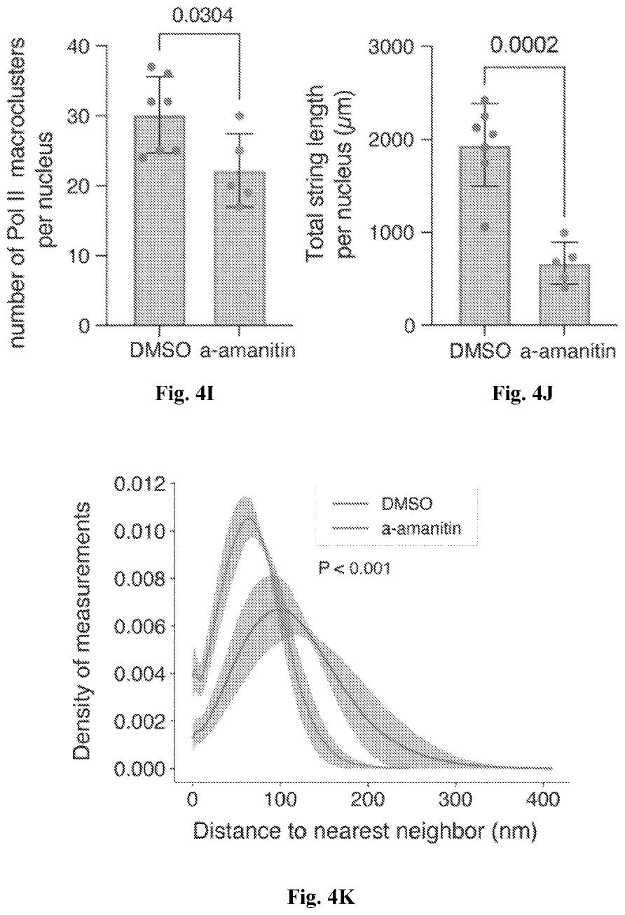

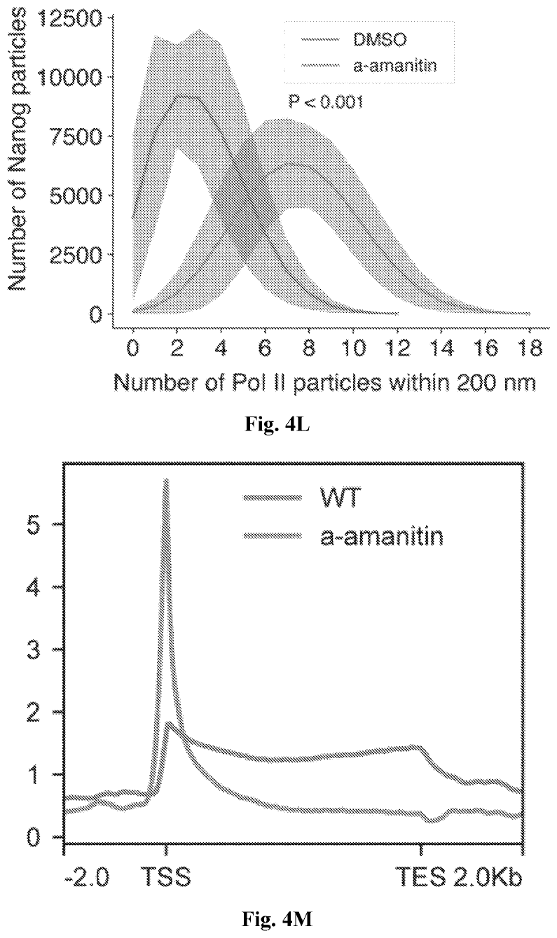

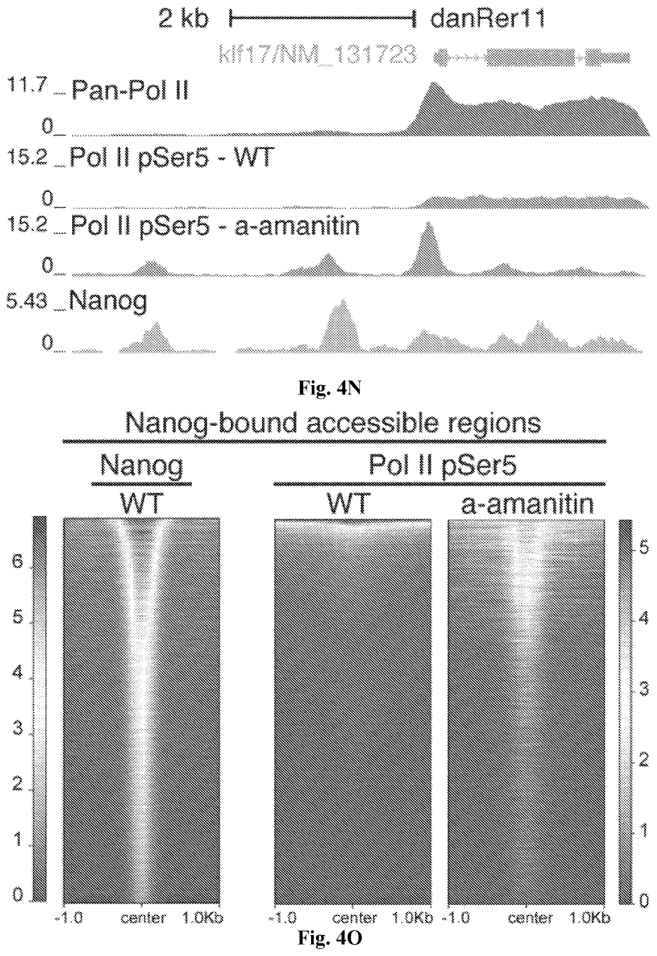

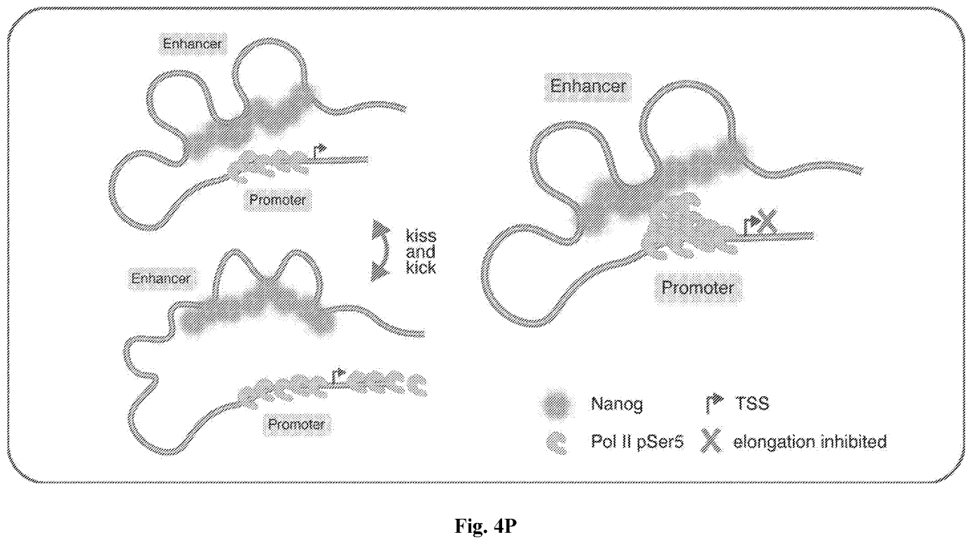

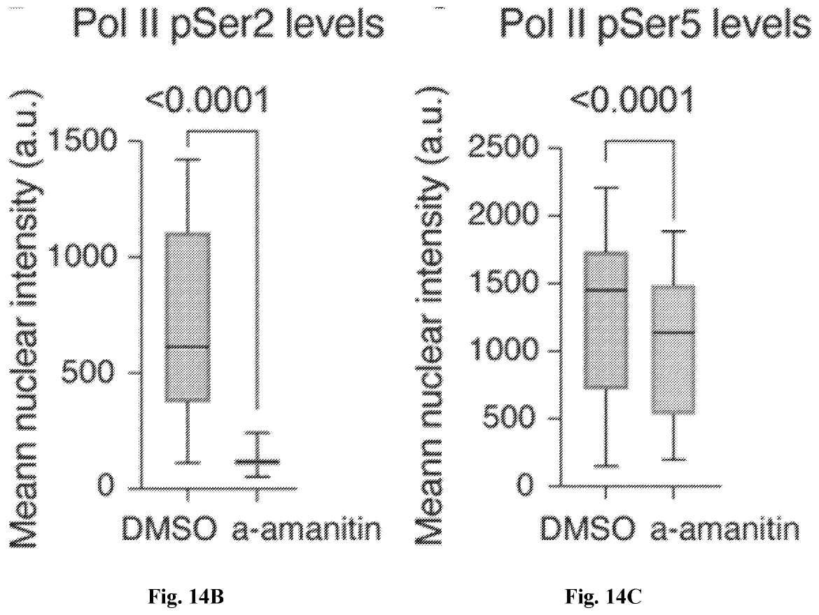

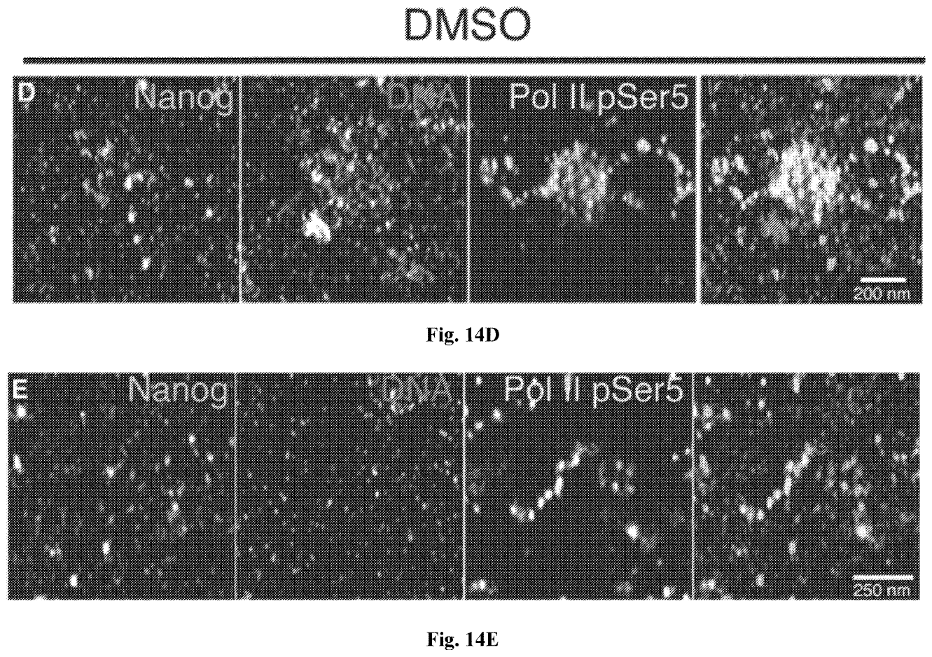

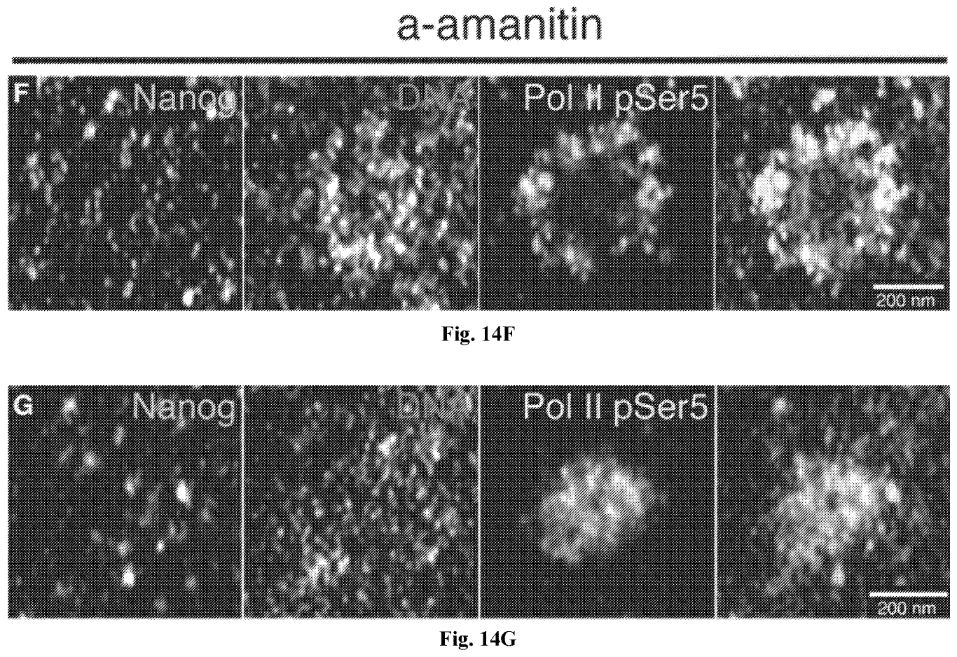

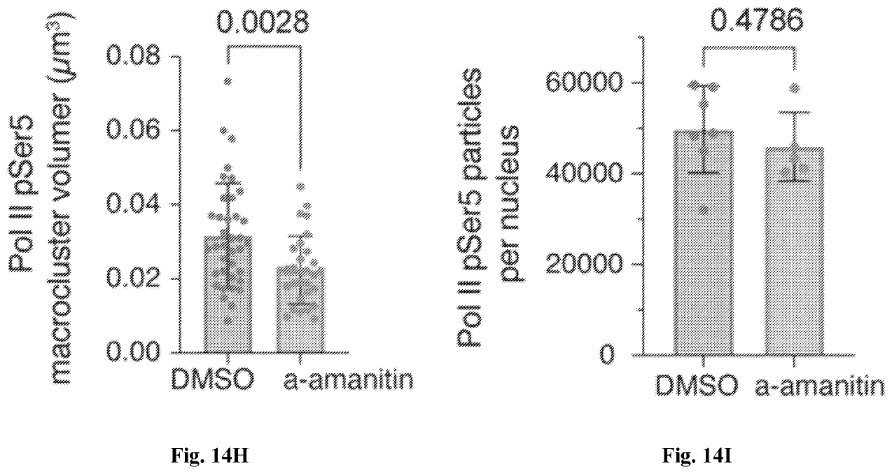

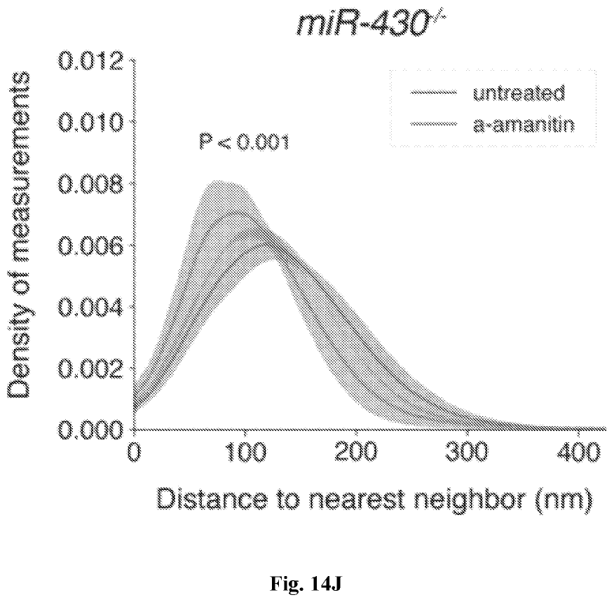

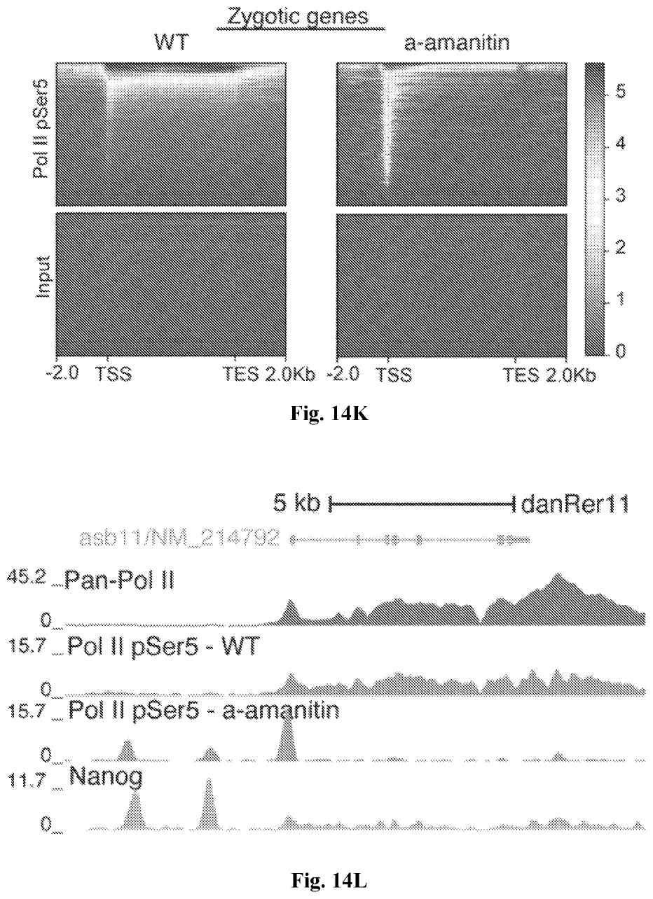

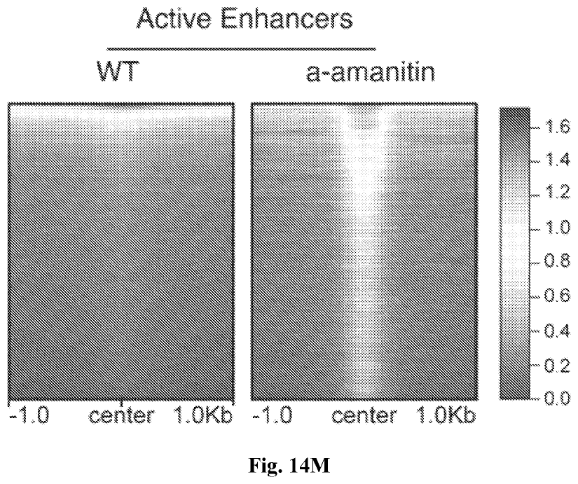

To test whether these distinct Pol II pSer5 structures formed in a transcription-dependent manner or whether they could be formed by Pol II recruitment independent of active transcription at 4 hpf, the present study inhibited transcription elongation with α-amanitin (FIGS. 4A-4H, 14A-14G), a pharmacological inhibitor that binds Pol II and prevents the conformational changes necessary for elongation. Treatment with α-amanitin led to a mild, but significant, reduction in the occurrence and size of Pol II macroclusters (26.3% reduction in the number of macroclusters; P=0.0304) (FIGS. 41 and 14H) but did not significantly reduce the overall particle count (FIG. 14I). In contrast, Pol II pSer5 strings were substantially reduced when transcription elongation was inhibited, consistent with their role in transcriptional elongation (66% reduction in total string length per nucleus; P=0.002) (FIGS. 4C, 4D, 4G, 4H, and 4J). Taken together, these data indicate that Pol II pSer5 strings represent transcription elongation, while Pol II pSer5 macroclusters may function as a regulatory hub formed independent of elongation.

Supporting this notion, the present study observed that Nanog and Pol II particles were closer after treatment with α-amanitin (FIG. 4K; median distance 106 nm vs 66 nm; P<0.001), and there were more Pol II pSer5 particles surrounding each Nanog particle (FIG. 4L; 3.1 vs 7.6 Pol II per Nanog; P<0.001). Similar behavior between Nanog and Pol II is observed when elongation is inhibited in miR-430^{-/-} embryos (FIG. S10J), suggesting that these changes occur at multiple loci in the genome. The present study explains these results with the following model: Nanog first recruits Pol II to enhancer-promoter hubs, bringing them into close proximity, then Pol II exits the regulatory hub in the form of Pol II pSer5 strings, transcribing the gene body (FIG. 4). When transcription elongation was inhibited, the present study observed an increase in the stoichiometry and proximity of Nanog-Pol II particles, consistent with Pol II stalling at the promoter and the stabilization of enhancer and promoter interactions.

To test this model, the present study performed ChIP-Seq for Pol II pSer5 in wild-type and α-amanitin treated embryos and observed extensive pileup of Pol II at gene promoters in the α-amanitin treated embryos (FIGS. 4M and 14K). As predicted from the ChromExM results, the present study also found Pol II pSer5 accumulating at Nanog-bound enhancers and other accessible sites (FIGS. 4N, 4O, 14L, and 14M). These regions are not enriched for RNA Pol II in the wild-type condition (FIGS. 4N, 4O, 14L, and 14M). These results can be explained by a model where, in wild-type embryos, enhancer-promoter contacts are transient, consistent with the kiss-and-run model, and there is, therefore, a larger mean distance between Nanog and Pol II (FIG. 4K). However, in the absence of transcriptional elongation, Pol II is continuously brought to the promoters and remains in close proximity to the enhancers as observed by ChIP-seq. This is further supported by the increase in the number of Pol II particles surrounding each Nanog particle (FIG. 4L). This is consistent with a transient interaction between enhancers and promoters proposed in the kiss-and-run model. The present study proposes a modified version of this model termed “kiss-and-kick,” where transcription itself kicks away the enhancer from the promoter as Pol II elongates (FIG. 4P). This would explain why a stabilization of Poll II in close proximity to Nanog bound enhancers when elongation is inhibited was observed.

### Example 4

Here, the present study developed ChromExM for multimodal super-resolution chromatin imaging. ExM physically enlarges biological samples to provide nanometer resolution without the need for specialized optical setups. The present study addressed concerns regarding the use of ExM to study nuclear processes by developing specific ChromExM-based assays to test the isotropy and positional stability of chromatin during the expansion process and an improved metabolic labelling strategy for chromatin imaging (FIGS. 2A-2G). These advances provide a significant increase in the resolution achieved by ExM applications for chromatin imaging (~3-15 nm vs ~65 nm) bringing it into a range where single nucleosomes were resolved. Previous methods to visualize chromatin, such as ChromEMT, lack multimodal labelling, while the resolution of single-molecule localization microscopy is limited by the size of the fluorescent labels (~20 nm for primary and secondary antibody), which becomes negligible in ChromExM given labels are applied after expansion. ChromExM provides significant technical advances for super-resolution chromatin imaging. The technology is advantageously combined with bright and photostable fluorescent labelling: chemical fixation; and high numerical aperture and long working distance objectives to achieve optimal resolution.

The present study used ChromExM to characterize the nanoscale organization of the pioneer factor Nanog and RNA Pol II as they activate transcription during genome activation. While previous studies have shown how pioneer factors direct chromatin opening in vitro and organize into hubs in vivo, the nanoscale organization of such hubs has remained unclear. Using ChromExM, the present study revealed that Pol II shows three types of organization during genome activation and is intimately associated with Nanog (FIGS. 3H-3K). Previous reports demonstrated colocalization between transcriptional activators and Pol II. Similarly, TFs such as Oct4, Brd4, and Mediator have been shown to form clusters associated with super-enhancers. ChromExM allows to resolve that Nanog and Pol II often occupy distinct regions within transcription hubs, consistent with their binding at enhancers and promoters that are in close contact.

How enhancers and promoters are organized to activate transcription is central to understanding gene regulation. Previous reports have concluded that enhancer-promoter contact is correlated, anticorrelated, or unrelated with transcription, leaving the question of how enhancer-promoter contact is related to transcription unanswered. Three models have been proposed to explain how enhancers and promoters interact to control gene expression: (i) stable contact between enhancers and promoters, (ii) a dynamic kissing model where enhancers and promoters transiently come into contact, and (iii) a TF activity gradient where TFs diffuse from enhancers to promoters rather than requiring physical contact.

The results herein are not consistent with models (i) and (iii), and lead to a model where Nanog can form clusters at enhancers, coming into physical proximity with Pol II at the promoter, eventually triggering transcription, which the present study observed outside of this shared regulatory hub as strings of Pol II. Consistent with this model, the present study found that Pol II strings were substantially reduced when elongation was inhibited, coinciding with an accumulation of Pol II at promoters and in Nanog-bound enhancers, indicating that the enhancer and promoter were stabilized in close contact (FIGS. 4A-4P). Taken together, these results indicate that Pol II elongation displaces enhancer-promoter contacts and lead us to propose a modified version of the kiss-and-run model the present study termed ‘kiss-and-kick.’ In this model, the dynamic association between the enhancer and promoter are kicked apart during elongation. This effect can be caused by either transcription elongation, or the nascent RNAs which have been shown to dissolve Mediator1 condensates. Kiss-and-kick could also explain transcriptional bursting, as elongation would be triggered in intervals while the enhancer and promoter are in contact, and then paused after elongation kicks away the enhancer.

Broadly speaking, the methodology presented here can be used to advance multiple fields focused on nuclear function, such as DNA damage and repair, DNA replication, epigenetic control of gene expression, genome structure, and how sequence variation affects chromatin structure and function in disease contexts.

### Example 5: Materials and Methods

### Zebrafish Embryo Production

Zebrafish embryos were obtained by randomly selecting and mating adult zebrafish of the TU-AB and TL strains. Zebrafish adults and embryos were maintained at 28° C. For all experiments, adults were allowed to mate for no more than 10 minutes to ensure embryos within each clutch developed synchronously. Embryos were obtained from multiple parents in all experiments unless otherwise noted.

### Embryo Injections and Treatments

All injections were performed by injecting 1 nL of nuclease-free water containing various reagents at the one-cell stage following dechorionation with Pronase.

Constructs and mRNA Synthesis

Nanog-llama was generated by cloning the eGFP-binding llama-tag nanobody into the c-terminal end of Nanog in the pCS2 vector. To control for sub-nuclear localization of the llama-tag, 3xNLS-llama was synthesized as a gBlock (IDT) and inserted into the pCS2 vector (FIG. 5C). Nanog-mEmerald was generated by cloning a zebrafish codon-optimized form of mEmerald (synthesized as a gBlock: IDT) into the c-terminal end of Nanog in the pCS2 vector. Nanog-HaloTag was cloned by swapping the mEmerald of Nanog-mEmerald for HaloTag. Nanog-Myc was cloned by inserting a 6xMyc tag into the c-terminal end of Nanog. Nanog DNA binding mutants (DBM) were generated by inserting the mutated homeodomain (synthesized as a gBlock; IDT) into the Nanog-mEmerald or Nanog-Myc plasmids. Homeodomain mutations were made based on previous reports. The present study assessed the functionality of Nanog DBM constructs by attempting to rescue the phenotype of MZnanog^{-/-} embryos, which arrest at the sphere stag (FIG. 5G). The RNA Pol II pSer2 mintbody was codon optimized for zebrafish expression, synthesized as a gBlock (IDT) and cloned into the pCS2 vector. mRNAs were synthesized from linearized plasmids using the mMessage mMachine SP6 in vitro transcription kit (Invitrogen, catolog no. AM1340) according to the manufacturer's instructions.

### Live Imaging

For Nanog live imaging using the llama-tag system (Bothma et al., Cell 173, 1810-1822.e1816 (2018)), transgenic Tg(Ubi-Tirl-P2A-GFP) fish with maternally supplied ubiquitous GFP expression were generated by Tol2-mediated transgenesis. Transgenic embryos were injected with 25 pg Nanog-llama mRNA and 0.5 pmol miR-430 molecular beacon (Gene Tools) in a separate needle or 25 pg llama-tag 3xNLS as a control (FIG. 5C). The present study observed that Tg(Ubi-Tirl-P2A-GFP) embryos did not exhibit nuclear enrichment for GFP when uninjected (FIG. 5C). Alternatively, Nanog live-imaging was performed by injecting wild-type (WT) embryos with 25 pg Nanog-mEmerald (FIG. 5E) or Nanog-HaloTag. For live imaging with the Pol II pSer2 mintbody, embryos were injected with 125 pg Pol II pSer2 mintbody mRNA and 25 pg Nanog-HaloTag mRNA. HaloTag labelling was performed by incubating embryos in 200 nM JF646 or JFX650 HaloTag ligand in system water for approximately 30 minutes beginning at the 8-cell stage. Imaging of Nanog DNA-binding mutants was performed by injecting WT embryos with 25 pg mRNA for each construct. To ensure Nanog-DBM constructs were expressed at similar levels, sibling embryos injected with the same needle were subjected to Western blotting (FIG. 5F). During live imaging, embryos were mounted in 0.8-1% low-melting point agarose in system water on a no. 1.5 coverslip. Images were acquired using a Zeiss LSM 880 or a Zeiss LSM 980 using Airyscan2 with a 20×0.8 NA air or 40×1.2 NA water immersion objective and a typical Z-step size of approximately 0.5-1 μm.

### Unexpanded Fixed Imaging

Staining was carried out as previously described (Chan et al., Developmental Cell 49, 867-881.e868 (2019)). Embryos were fixed in 4% paraformaldehyde (PFA) at 4° C. overnight, washed with 0.5% Trition-X-100 (PBSTr), then dehydrated in series of methanol/PBSTr washes and stored at −20° C. Embryos were rehydrated with a series of methanol/PBSTr washes and blocked for 2-3 hours at room temperature (RT) in 10% bovine serum albumin (BSA) in PBSTr. Then, embryos were incubated at 4° C. overnight 10 with primary antibody (Pol II pSer2, Abcam abl93468, 1:500 or 1:1,000: Pol II pSer5, Abcam ab5408, 1:1,000) in 10% BSA in PBSTr, washed with PBSTr, and incubated in secondary antibody (Goat anti-Rabbit-IgG—Atto 647N. Sigma-Aldrich, catalog no. 40839, 1:1,000; Goat anti-Mouse-IgG —Atto 550. Rockland, catalog no. 610-154-121, 1:1,000) in 10% BSA in PBStr for two hours at RT. Click reactions were performed with 5 μM AZ555 picolyl azide (Click Chemistry Tools, catalog no. 1288-1) or AZ568 picolyl azide (Click Chemistry Tools, catalog no. 1292-1) using the Click and Go cell reaction buffer kit (Click Chemistry Tools, catalog no. 1263) following the manufacturer's instructions with the exception that the click reaction was performed for 1 hr at RT. Embryos were mounted in 1% low-melting point agarose in PBS and imaged with using a Zeiss LSM 980 with Airyscan2 and a 40×1.2 NA water immersion objective and a typical Z-step size of 0.5 μm or less.

### Western Blot

Embryos were injected with 25 μg of mRNA encoding Nanog-mEmerald, Nanog-DBM-mEmerald constructs, or were uninjected. Injected embryos were either subjected to live-imaging or Western blot. At 4 hpf, embryos were manually deyolked with forceps and then flash-frozen with liquid nitrogen and optionally stored at −80° C. until further processing. Samples were subsequently lysed with sample buffer (1× NuPAGE LDS Sample Buffer supplemented with dithiothreitol (DTT)) by pipetting up and down several times and then boiled for 10 min at 95° C. Next, 35 μL of each lysate (~10 embryos) was loaded and run on a NuPAGE gel (4-12% Bis-Tris, Thermo Fisher Scientific) and transferred to a nitrocellulose membrane following instructions from the iBlot 2 Gel Transfer Device (Thermo Fisher Scientific). Membranes were blocked in blocking buffer (5% skim milk in PBS with 0.1% Tween-20 (PBST)), incubated overnight at 4° C. with primary antibody diluted in blocking buffer (GFP. Thermo Fisher Scientific, catalog no. A11122, 1:1,000: Actin, Sigma-Aldrich, catalog no. A5060, 1:2,000), washed with PBST, and then incubated with a HRP-coupled secondary antibody diluted in blocking buffer (Abcam, catalog no. ab6721, 1:10,000). SuperSignal West Pico PLUS Chemiluminescent Substrate (Thermo Fisher Scientific, catalog no. 34580) was used for protein detection.

### Metabolic Labelling, RNA Injection, and Drug Treatment

For ChromExM, embryos were injected at the one-cell stage with 3.5-4 pmol f-ara-EdU (Click Chemistry Tools, catalog no. 1403-5) for DNA labelling unless otherwise noted. For metabolic RNA labelling, embryos were injected at the one-cell stage with 50 pmol 5-Ethynyl Uridine (EU) (Invitrogen, catalog no. C10329). Labeling persisted during development until sample collection. For Nanog imaging, embryos were injected with 25 pg Nanog-6xMyc mRNA. To inhibit transcription elongation, embryos were injected with 0.2 ng α-amanitin (Sigma-Aldrich, catalog no. A2263).

### ChromExM—Gelations and Expansion

To perform ChromExM, the present study adapted the gelation processes previously developed for pan-ExM (M'Saad et al., 2020, Nature Communications 11). Embryos were manually deyolked in deyolking buffer with calcium (55 mM NaCl, 1.8 mM KCl, 1.25 mM NaHCO₃, 2.7 mM CaCl₂)) to preserve the integrity of the animal cap while removing the yolk. After rinsing in PBS, embryos were fixed with EM-grade fixatives for 15 min at RT with rotation in 3% PFA (Electron Microscopy Sciences, catalog no. 50-980-487) and 0.1% glutaraldehyde (GA; Ted Pella Inc., catalog no. 18420) in PBS. Embryos were washed 1-3× with PBS and then post-fixed in 1% acrylamide and 0.7% PFA at 37° C. with shaking for 6-8 hours to enable direct protein anchoring to the first hydrogel. Next, embryos were washed with PBS and then manually mounted on a round no 1.5 12 mm coverslip in a gelation chamber assembled from two sets of two no. 2 22 mm square coverslips superglued onto a glass slide on either side of the 12 mm no. 1.5 round coverslip. The first gelling solution comprised 10% acrylamide (Supelco, catalog no. 01697-500ML; RPI, catalog no. A11265-1000.0), 0.1% N,N′-(1,2-dihydroxyethylene)bisacrylamide (DHEBA: Millipore-Sigma, catalog no. 294381-5G), and 19% sodium acrylate (SA; Santa Cruz Biotechnology, catalog no. sc-236893C). Each lot of SA was checked for purity and was prepared as a 38% stock solution and centrifuged at 4,000 rpm for 5 min to remove any impurities before use. To activate the first gelling solution, ammonium persulfate (APS; Sigma-Aldrich catalog no. A3678-25G) N,N,N′,N′-tetramethylethylenediamine (TEMED: American Bio. catalog no. AB02020-00050) were added to the first gelling solution at a final concentration of 0.05% each immediately before use. The activated gelling solution was applied to the mounted embryos, embryos were manually repositioned on the coverslip, and another 22 mm coverslip was used as a lid for the gelation chamber to give an initial gel thickness of ~220 μm. The assembled gelation chambers were placed in a humidified gas exchange chamber to replace the air with nitrogen and then incubated at 37° C. for 1.5 hrs. After the first gelation, the gelation chambers were removed and individual embryos were isolated from the polymerized first gel using a 1 mm tissue biopsy punch. Embryos were then denatured for 1-2 hours at 76° C. with shaking in denaturation buffer (200 mM NaCl, 50 mM Tris, 200 mM sodium dodecyl sulfate, pH 6.8). After denaturation, gels were washed extensively with PBS and then transferred to ultrapure water for two 30 min washes, and then left to expand overnight in fresh ultrapure water.

After the first expansion. embryos were re-embedded in a neutral hydrogel to maintain the embryos in their expanded state while embedding in the second swellable hydrogel. The neutral gelling solution consisted of 10% acrylamide and 0.05% DHEBA. To trigger polymerization, APS and TEMED were added to a final concentration of 0.25%. Embryos were washed in this activated neutral gelling solution three times for 20 minutes, using freshly activated gelling solution for each wash. After the final wash, the gels were sandwiched between two no. 1.5 coverslips, transferred to a humidified gas exchange chamber, and purged with nitrogen. Gelation then proceeded for two hours at 37° C. After gelation was complete, the gels were retrieved and stored in PBS overnight.

Next, embryos were embedded in a second swellable hydrogel consisting of 10% acrylamide, 19% SA, and 0.1% N,N′-methylenebis(acrylamide) (BIS; Alfa Aesar, catalog no. J66710-14) in PBS. The gelling solution was activated by the addition of 0.25% APS and TEMED. Embryos were washed three times for 15 minutes on ice in this activated gelling solution. After the last wash, the gels were sandwiched between two no. 1.5 coverslips, transferred to a humidified gas exchange chamber, and purged with nitrogen. Gelation then proceeded for two hours at 37° C. After gelation was complete, the gels were retrieved and incubated in 200 mM NaOH for 1 hr to dissolve the DHEBA crosslinks of the first two gels. Gels were then neutralized by washing three times for 30 minutes with excess PBS and processed for staining. During the final PBS wash, excess gel was trimmed from the embryos to limit gel volume during staining. After all staining was complete, gels were expanded a final time in ultrapure water, which was exchanged once an hour for two hours, and then left to expand completely in fresh ultrapure water for at least 6 hours in the dark.

For confocal and STED microscopy, gels were manually sliced into thin sections using a razor blade to ensure nuclei were within the working distance of the objective and mounted in no. 1.5 50 mm glass bottom MatTek dishes (MatTek, catalog no. P50G-1.5-30-F) and sealed with Picodent Twinsil or Picodent Eco-sil (Leica Microsystems, catalog no. 11532691) after placing a no. 1.5 coverslip on top of the gels. After mounting, gels were stored at room temperature in the dark and imaged within one week.

### ChromExM DNA Labelling

DNA labelling was performed just prior to the final expansion. Initially, SYTOX Green (Invitrogen, catalog no. S7020; diluted 1:5,000 in Hanks' Balanced Salt Solution (HBSS), Gibco, catalog no. 14170112) was used to label DNA and localize the nuclei. However, after observing extensive photobleaching (FIG. 6C), the present study identified alternative DNA labelling approaches. Specifically, the present study utilized the injection of an alkyne modified nucleoside, f-ara-EdU, to allow brighter and more photostable azide modified dyes to be covalently added to the DNA through click chemistry. Click reactions were performed using the Click and Go cell reaction buffer kit with 5-8 μM dye according the manufacturer's instructions with the exception that the reaction was performed for 1 hr. The present study found AZ555 picolyl azide provided ~45× brighter labelling of the DNA compared to AZ555 azide (Click Chemistry Tools, catalog no. 1287-1) (FIGS. 6D and 6E) and thus used picolyl azides (coupled to AZ555 or AZ568) for imaging when possible.

### ChromExM RNA Labelling

Nascent RNAs were labelled by injection of EU and detected by click labelling with 5-8 μM AZ555 or AZ568 picolyl azide as described for DNA labelling.

### ChromExM Immunostaining

All antibodies were used at 1:250 dilutions and were applied to samples in 2% BSA in PBST. After gels were neutralized by PBS washing following the NaOH incubation, samples were incubated in primary antibody (Pol 11 pSer5. Abcam ab5131; Myc, Cell Signaling Technology #2276S; H3, Abcam ab1791) for 40-50 hours at room temperature, then washed 3×20 minutes in PBST. Next, samples were incubated with secondary antibodies coupled to ATTO647N (Goat anti-Rabbit-IgG—Atto 647N, Sigma-Aldrich, catolog no. 40839; Goat anti-Mouse-IgG—Atto 647N, Sigma-Aldrich, catalog no. 50185) or Alexa 488 (Goat anti-Rabbit IgG—Alexa 488. Invitrogen, catalog no. A-11008: Goat anti-Mouse IgG—Alexa 488, Invitrogen, catalog no. A32723) for 60-70 hours at room temperature, then washed 3×20 minutes in PBST. In some instances, the gels were manually halved before staining to improve antibody uptake. After immunostaining, any additional staining, such as Click labelling, was performed and then the samples were expanded for the final time.

### HCR RNA FISH

Hybridization chain reaction RNA fluorescent in situ hybridization (HCR RNA FISH) was performed using standard kits with custom designed probes targeting miR-430 (Molecular Instruments). In total, 22 probes were designed to target a 1.2 kb transcribed region between CAGE-Seq peaks in the miR-430 locus. For unexpanded embryos, HCR RNA FISH was performed following the manufacture's protocol for zebrafish embryos. When combined with immunostaining, immunostaining was performed prior to HCR RNA FISH as described above with the exception that Antibody Buffer (Molecular Instruments) was used for blocking and antibody staining instead of 10% BSA in PBSTr. When combined with ChromExM, the HCR RNA FISH protocol was performed as described for unexpanded embryos with the following modifications. Antibody staining was performed prior to HCR RNA FISH as described above for ChromExM but Antibody Buffer (Molecular Instruments) was used instead 10 of 2% BSA in PBST. Probes were used at 16-32 nM final concentration and incubated at 37° C. for 44-52 hours. For amplification, 240-480 nM each of hairpin H1 and hairpin H2 coupled to a 488 fluorophore were incubated with the samples for 16-24 hours at RT. After washing with 5×SSCT (5× sodium chloride sodium citrate (diluted from 20×SSC, Sigma-Aldrich catalog no. S6639-1L), 0.1% Tween-20) for 3×20 min, Click reactions were performed for DNA labelling 15 and samples were expanded and mounted as described above.

### Photocleavable Biotin Labelling and Photocleavage

Embryos injected with f-ara-EdU were subjected to the first gelation as described above. Immediately after the first gelation, embryos were permeabilized in PBS containing 0.1% saponin (PBS-s). Then, 5 μM photocleavable (PC) biotin azide (Click Chemistry Tools, catalog no. 1119-1) was clicked on to the EdU as described for fluorescent azide labelling with the addition of 0.1% saponin to the click reaction. Embryos were subsequently stained with SYTOX Green (1:5,000 in HBSS with 0.1% saponin) to visualize DNA during photocleavage. Gels were then washed three time in PBS-s and stored at 4° C. until photocleavage. Gels were mounted in 1% low-melt agarose on no. 1.5 coverslips and the biotin moiety was cleaved by targeted illumination with ~1.5 mW/cm2 405 nm light for 5,000-10,000 cycles on a Zeiss LSM 980 using a 40×1.2 NA water immersion objective. After cleavage, the released biotin was washed out in PBST and embryos were then denatured and processed following the standard ChromExM method described above. Biotin-labelled DNA was detected using 1:250 Neutravidin-DyLight 550 (Invitrogen, catalog no. 84606) or Neutravidin-DyLight 594 (Invitrogen, catalog no. 22842) in 2% BSA in PBST for 1 hr at room temperature. DNA was counterstained using SYTOX Green (1:5,000 in HBSS). For unexpanded samples, staining and cleavage were carried out as above except that Neutravidin was used at 1:500 in 10% BSA in PBSTr and the final SYTOX Green staining was performed in the presence of 0.1% saponin. Expanded samples were imaged 35 in LSM mode on a Zeiss LSM 980 using a 20×0.8 NA or 10×0.3 NA air objective. Unexpanded samples were imaged using Airyscan2 on a Zeiss LSM 980 with a 40×1.2 NA water immersion objective. Expanded images are shown as a maximum intensity projection of several Z planes to better show the striped pattern. Unexpanded images are shown as a maximum intensity projection of the entire nucleus.

### Nucleosome Array Preparation and Imaging

Nucleosome arrays were assembled as described in Frederick et al. (Nature Structural & Molecular Biology 30, 31-37 (2023)) and Cirillo et al. (Mol Cell 9, 279-289 (2002)). Assembly reactions containing 2 g of end-labeled 2.7 kb Cx3crl DNA fragments (Frederick et al., 2023, Nature Structural & Molecular Biology 30, 31-37) and core histones at a 1:1.0 molar ratio of octamers to nucleosomal sites, and 2 M NaCl in a total of 10 μl were incubated at 37° C. for 15 min, serially diluted by adding 3.3, 6.7, 5, 3.6, 4.7, 6.7, 10, 30, and 20 μl of 50 mM HEPES (pH 7.5), 1 mM EDTA, 5 mM DTT, and 0.5 mM PMSF in 15 min incubation steps at 30° C., and brought to 0.1 M NaCl by adding 100 Ed of 10 mM Tris-HCl (pH 7.5), 1 mM EDTA, 0.1% IGEPAL, 5 mM DTT, 0.5 mM PMSF, and 20% glycerol, followed by incubation at 30° C. for 15 min. Array saturation was determined by EcoRI (New England Biolabs catalog no. R3101L) digestion, performed with a concentration of 2 nM nucleosome array or free DNA in 20 mM HEPES (pH 7.5), 50 mM KCl, 1% glycerol, 5 mM DTT, 100 μg/ml BSA, and 2 U/μl EcoRI. Following a 2 hour incubation at 37° C., digestion products were resolved on 4% polyacrylamide 0.5×TBE gels and analyzed as described in Cirillo et al., 2002, Mol Cell 9, 279-289.

Nucleosome arrays were subjected to ChromExM by either fixing 7 nM arrays in 3% PFA and 0.1% GA in PBS for 5 min at RT and post-fixing in 1% acrylamide and 0.7% PFA or directly anchoring 7 nM arrays in post-fix (1% acrylamide and 0.7% PFA) solution for 6-8 hours at 37° C. Thus, arrays were post-fixed in a buffer containing 1% acrylamide, 0.7% PFA, 59.25 mM NaCl, 0.675 mM KCl, 2 mM Na₂HPO₄, 0.5 mM KH₂PO₄, 12.5 mM Hepes, 2.5 mM Tris-HCl, 0.5 mM EDTA, 0.05% IGEPAL, 5% Glycerol, 2.5 mM DTT, and 0.25 mM PMSF. Gels were assembled following the same procedure described for embryos with the following modifications: the gelation chamber used a 5 mm coverslip instead of 12 mm coverslip to produce a smaller initial gel diameter; the gelation chamber was assembled using two no. 1.5 coverslips to produce an initial gel thickness of ~175 μm; and the gelling solution was activated with a final concentration of 0.125% APS and TEMED to enable gelation to proceed without the addition of nitrogen gas. The second and third gelation steps were performed as described for embryos. Expanded arrays were stained with anti-H3 antibody as described for embryos with the exception that samples were incubated in primary antibody for ~36 hours at RT and secondary antibody for ~24 hours. SYTOX Green (1:3,000 in HBSS) was used visualize the array DNA. Expanded arrays were imaged as described for expanded embryos. Nucleosomes in each array were counted manually.

Nucleosome arrays were visualized by electron microscopy using negative staining. Briefly, 7 nM arrays were fixed and dialyzed as previously described (Panchenko et al, 2011, Proceedings of the National Academy of Sciences 108, 16588-16593) in 10 mM Hepes, 0.25 mM EDTA 2.5 mM NaCl, and 0.1% glutaraldehyde. Following overnight dialysis, arrays were applied to carbon coated copper grids, which were glow discharged immediately prior to addition of the arrays. Solution was incubated at RT for 30 seconds. Filter paper was used to remove excess liquid and 1% uranyl acetate (UA) was added. After 30 seconds, excess liquid was again removed and grids were rinsed 3× in ultrapure H₂O and air dried. Imaging was performed on an FEI Biotwin Transmission Electron Microscope (TEM) operated at 80 kVs. Nucleosomes in each array were counted manually.

### Confocal Microscopy of Expanded Samples

Images were acquired using a Zeiss LSM 980 with Airyscan2 in airyscan mode to improve acquisition speed unless otherwise noted. Gels were imaged with either a 10×0.3 NA air objective to obtain overview images of the sample, or with a 40×1.2 NA water immersion objective to match the optical properties of the aqueous gel. Z-stacks were taken using a 0.5 μm step size which is an effective step size of ~30 nm when accounting for the average expansion factor of ~15. For 2.5 hpf embryos, a Z-step size of 0.5-0.75 μm was used. XY pixel size was set between approximately 80-110 nm (typically 100 nm; ~6.7 nm after correcting for the expansion factor). Images were acquired using bidirectional scanning and 2-4× frame averaging. Airyscan images were processed in ZenBlue (version 3.3, 3.5, or 3.6) using default 3D settings. Fluorophores were excited using 488 nm, 561 nm, and 639 nm laser lines. Multi-channel images were acquired sequentially. When imaging dyes excited by two or more consecutive laser lines, bandpass filters were applied during detection to eliminate spectral bleed through. In some cases, channels were aligned using ZenBlue or FIJI to correct for chromatic aberrations. Images are shown as single Z planes unless otherwise noted and, in some cases, images were smoothed with a median filter or gaussian blur for display.

### STED Microscopy of Expanded Samples

5 STED microscopy was performed on a Leica SP8 3×Gated STED microscope using an 86×1.2 NA water immersion objective and 9-45 nm XY pixel size (0.6-3 nm after correcting for the expansion factor). DNA was stained with SYTOX Green and H3 was labelled with ATTO 647N (primary and secondary antibody at 1:100) as described elsewhere herein. ATTO 647N was excited with a white light laser set to 651 nm for ATTO 647N. A 775 nm depletion laser was used at 50-100% power, 2D Images were acquired with line averaging and, in some cases, frame accumulation. In some images, the STED beam alignment appeared to drift after initial alignment and was corrected in the images after acquisition using a rigid transformation in FIJI.

### Estimation of the Gel Mesh Size

To estimate the gel mesh size, the first hydrogel was polymerized as described above in the presence of 6.25 μM H1-Alexa 488 (Invitrogen, catalog no. H13188), 3,000 MW lysine-fixable TMR-dextran (Invitrogen, catalog no. D3308), or 2,000,000 MW lysine-fixable TMR-dextran (Invitrogen, catalog no. D7139) without cross-linking to the gel to observe their mobility. Lysine-fixable dextrans were used to more closely match the properties of a protein, such as H1, and control for potential interactions with the gel. After polymerization was complete, gels were cut into pieces, hydrated with PBS, mounted between two no. 1.5 coverslips, and sealed with Picodent Twinsil. FRAP was performed on a Zeiss LSM 980 using a 20×0.8 NA objective. Images were acquired every 2 seconds for 200 seconds and bleaching was performed after five frames were acquired to establish the baseline. FRAP was performed on three distinct regions of the gel for each experiment. The diffusion coefficient was calculated according to Soumpasis (Biophysical J., 95-97 (1983)) and Kang et al. (Traffic 13, 1589-1600 (2012)).

### Image Analysis

Image analysis was performed using FIJI (Schindelin et al., 2012. Nature Methods 9, 676-682) and using custom python scripts for computing distances between particles. The 3D rendering shown in FIG. 8B, right panel, was generated using Napari (Sofroniew et al., 2022, Napari: a multi-dimensional image viewer for Python). In some cases, figures were generated using QuickFigures (Mazo, 2021, PLoS One 16, e0240280) and other 3D renderings were generated using Imaris (v10.0; Oxford Instruments).

### Live Imaging

From live imaging data, individual timepoints were used to identify Nanog foci. First, nuclei were segmented by applying a median filter and then thresholding using the Otsu or Li method. Individual nuclei were identified by connected components labelling using MorphoLibJ (Legland et al., 2016, Bioinformatics 32, 3532-3534) and their morphological and intensity characteristics were measured. Nanog foci were segmented by first applying background subtraction to the raw image and then 3D watershed segmentation (3D ImageJ Suite: (Ollion et al., 2013, Bioinformatics 29, 1840-1841). Individual foci were identified using connected components labelling in MorphoLibJ and small background objects arising from segmentation error were removed using a minimum size threshold for objects. The intensity and morphological characteristics of the foci were quantified using MorphoLibJ. The relative enrichment of Nanog in the foci was determined by measuring the intensity of each foci compared to the mean nuclear intensity in the same channel, miR-430 transcription was identified by segmented the miR-430 transcription sites using the same approach as for Nanog foci and then the number of miR-430 transcription sites containing Nanog foci were identified. For analysis of the Pol II pSer2 mintbody, images were first maximum intensity projected and corrected for drift using the Template Matching plugin (Tseng et al., 2011, Lab on a Chip 11, 2231-2240). Then, the intensity within an ROI surrounding Nanog foci was measured for both channels over time.

### Unexpanded Fixed Imaging

Nuclei were segmented based on DNA staining by applying a median filter or gaussian blur and Otsu or Li thresholding. Individual nuclei were identified by connected components labelling with MorphoLibJ and the intensity in each channel was measured. Statistical tests were performed using GraphPad Prism.

### Expansion Factor Measurement

The expansion factor was measured by comparing the nuclear cross sectional area of unexpanded and expanded embryos of the same stage. First, nuclei were segmented by applying a median filter and then thresholding using the Otsu or Li method. Connected components labelling was performed using MorphoLibJ to identify individual nuclei and the area of each individual nuclei was measured at its midsection. Only nuclei with a circularity value >0.75 were included in analysis to exclude mitotic nuclei. A single correction factor reflecting the mean linear expansion factor of 15× was applied to all ChromExM images unless otherwise noted.

Quantification of Inter-Stripe Distances after Photocleavage

After rotating the images such that the stripes were perfectly vertical, photocleaved stripes were identified based on signal in the Neutravidin channel by applying a median filter followed by difference of gaussian filter. A manually-determined threshold was applied to the filtered image (Otsu or Li) to isolate the stripes. The binarized stripes were skeletonized using BoneJ (Doube et al., 2010, Bone 47, 1076-1079) and the longest shortest path of each skeleton was identified using Analyze Skeleton (2D/3D) (Arganda-Carreras et al., 2010, Microscopy Research and Technique 73, 1019-1029). The longest shortest path was used to represent the center of each stripe (FIG. 6C). The coordinates of each line were extracted by selecting the line using the Magic Wand tool and saving the XY coordinates under Analyze>Tools>Save XY Coordinates. Inter-stripe distance was calculated at each pixel of the stripe by measuring the difference in X values between neighboring stripes where they had matching Y values. To account for variation in stripe positioning, the present study calculated the relative inter-stripe distance by comparing the inter-stripe distance at each Y position with the mean inter-stripe distance for all Y positions.

To control for potential errors in segmentation of the stripes due to the heterogenous chromatin signal, the present study used the SYTOX Green image (bulk DNA stain) to generate a simulated control image with perfectly straight stripes with no edge distortion (FIG. 6C). This was achieved by generating rectangular ROIs over the true photocleaved stripes and applying this ROIs to the SYTOX Green image such that the intensity in these regions was reduced to half their initial value. These simulated stripe images were then processed in the same manner described above for the true photocleaved images. Any deviation in the inter-stripe distance in this simulated image must result from the segmentation method (e.g. due to the heterogenous chromatin signal). Thus, only those differences detected in the true photocleaved stripes that exceed those detected in the simulated images is a result of perturbations to chromatin organization. Statistical tests were performed using GraphPad Prism.

### Segmentation of ChromExM Immunostaining

For Nanog. Pol II pSer5, and H3 immunostaining after expansion, particles of each target were segmented using the following approach. First, the complete nucleus was segmented based on DNA staining as described above for unexpanded nuclei segmentation with the exception that segmentation was manually corrected as needed. The area outside of the nucleus was then masked and excluded from subsequent segmentation and analysis. Antibody signal was segmented by applying a 3D TopHat filter followed by 3D watershed segmentation (3D ImageJ Suite) and connected components labelling using MorphoLibJ. Small background objects arising from segmentation error were removed using a minimum size threshold for objects. Intensity and morphological characteristics of the objects were measured and their 3D centroid position was extracted (using MorphoLibJ) for subsequent analysis. For segmentation of Pol II pSer5 macroclusters, a median filter was applied to the raw image followed by Otsu thresholding and connected components labelling using MorphoLibJ with a minimum size cut-off. For segmentation of Pol II strings. Pol II pSer5 images were processed with median filter followed by Otsu thresholding. Strings were identified by skeletonization of the binary image (Analyze Skeleton (2D/3D)) (Arganda-Carreras et al., 2010, Microscopy Research and Technique 73, 1019-1029) and measurements were exported. String length was determined by summing the length of each branch for all skeletons. Subsequently, only strings with a length ≥200 nm after correcting for the expansion factor were considered bona fide Pol II pSer5 strings. Class 1 strings were identified as the top six longest strings and were manually validated. Particles belonging to Pol II pSer5 strings were identified by finding the string identity associated with each particle using MorphoLibJ.

### Spatial Relationships

Using the centroid position of segmented objects, the Euclidian 3D distance to the nearest neighbor was calculated for each object using the distance.cdist method of the spatial module from Scipy (83) and estimated as a kernel density function. To obtain the edge-edge distance between objects rather than centroid-centroid distance, the average radius of the segmented objects was determined in each experiment and subtracted from the centroid-centroid distance (negative values were zeroed). This was calculated as the radius of the largest inscribed sphere in each object using MorphoLibJ. All measurements are reported as edge-edge distance. The number of objects within a given 3D radius from each object was calculated by counting the number of objects within that radius by first applying a filter for the maximum distance and then using the bincount method from Numpy (Harris et al., 2020. Nature 585, 357-362). Plots were obtained using Matplotlib (Hunter, 2007, Computing in Science & Engineering 9, 90-95). All computations were performed using Python.

### Classification of Nanog-H3 Structures

Classes of Nanog-H3 were determined based on two criteria. First, the present study performed quantitative analyses of the spatial distribution between all Nanog and H3 particles and measured the distance to the nearest Nanog particle for all H3 particles (described in ‘Segmentation of ChromExM immunostaining‘ and’Spatial relationships’). This represents the point-to-set correlation and was obtained by calculating the minimum distances (i.e. nearest neighbor) from one set of points to the other. This represents the distance to the nearest Nanog particle for each H3 particle and vice versa. The present study measured this distance for all particles and have estimated it using a kernel density function to obtain a quantitative, global analysis of Nanog-H3 organization (FIGS. 8E-8F). Second, the present study then used this global quantification of nearest neighbor distance as a means of assigning distance-based criteria to define the three classes (cutoffs shown on the graph in FIG. 8F). Based on the visual observations of the images and understanding of the underlying biology, the present study manually determined the nearest neighbor distance cutoffs for each category. Class 1 required Nanog-H3 particles to be within 20 nm; Class 2 required Nanog-H3 to be within 20 nm as in Class 1, but with at least one additional Nanog particle within 50 nm of the H3 particle; Class 3 required H3 particles to be at between 20-100 nm away from any Nanog particle; and Class 4 required H3 particles to be at least 100 nm away from any Nanog particle.

### Classification of Pol II Structures

Classes of Pol II pSer5 organization were identified first by visual inspection of the images, which identified Pol II pSer5 strings, which represented a novel structure that could then be quantitatively identified and measured using image segmentation (see ‘Segmentation of ChromExM immunostaining). Upon inspection of the length distribution of these strings, the present study noticed extremely long outliers consistent with visual inspection of the images (FIG. 3O). Thus, the present study divided Pol II strings into two classes: Class 1 being these extreme outliers, and Class 2 being all other strings. Class 3 was categorized as macroclusters, consistent with the Pol II pSer5 clusters observed in unexpanded images (FIGS. 5E and 14A). The present study quantitatively identified these Class 3 clusters using image segmentation based on their intensity and size characteristics (see ‘Segmentation of ChromExM immunostaining).

### Whole-Genome Sequencing and Assembly

For whole-genome long-read sequencing, haploid WT and miR-430^{-/-} embryos were generated as previously described (M. Westerfield, The zebrafish book: a guide for the laboratory use of zebrafish. http://zfin dot org/zf_info/zfbook/zfbk dot html, (2000)). Briefly, testes were isolated from six adult male fish of each genotype and sperm was suspended in 1 mL of full strength Hank's saline (0.137 M NaCl, 5.4 mM KCl, 0.25 mM Na₂H PO₄. 0.44 mM KH₂PO₄, 1.3 mM CaCl₂), 1.0 mM MgSO₄, 4.2 mM NaHCO₃). Then, 300 μL of the sperm suspension was transferred to a 60 mm plastic petri dish (with no lid), which was subsequently placed in a larger dish atop a ~2 cm thick layer of ice and then placed on the bottom of SPECTROLINKER XL-1500 UV crosslinker while still on ice. Sperm was irradiated with the UV crosslinker (energy: 250×100 μJ/cm²) and kept on ice while collecting eggs. Egg isolation and in vitro fertilization was performed as previously described (Westerfield). Embryos were raised to 2-3 days post fertilization, manually dechorionated, and single embryos were flash frozen in liquid nitrogen and stored at −80° C. DNA was isolated from individual haploid embryos using the Monarch Genomic DNA Purification Kit (New England Biolabs, catalog no. T3010S) following the manufacturer's instructions. PacBio library preparation and HiFi sequencing was performed by the Yale Center for Genome Analysis using a PacBio Sequel II system. In total, four individual haploid embryos (two WT and two miR-430^{-/-}) were sequenced, reaching up to 12× and 8× read coverage in WT and miR-430^{-/-} embryos, respectively.

To compute genomic coverage, the PacBio HiFi reads (1.97 and 0.84 million reads for WT replicates; 1.4 and 1.1 millions for miR-430−/− replicates) were aligned on the GRCz11 zebrafish genome using minimap2 (Li, Bioinformatics 37, 4572-4574 (2021)) with the parameters ‘-x map-hifi’ and ‘--secondary=no’. Genome-wide coverage profiles were obtained using GeneAbacus (https://github.com/vejnar/GeneAbacus) with the parameters ‘--profile_type all-slice’ and ‘--profile_multi 1000’ and imported into Numpy arrays in Python using GeneAbacus-python (https://github.com/vejnar/GeneAbacus-python) to compute average coverage.

HiFi reads from one WT embryo were assembled into 3,151 contigs (15,546 contigs for the second replicate) using hifiasm (Cheng et al, 2022, Nature Biotechnology 40, 1332-1335) with ‘-l 0’ parameter to disable duplication purging as haploid embryos were sequenced. Most contigs were linear sequences: however, hifiasm also produced 11 circular contigs (15 in the second replicate). To examine how contigs were created, the present study visualized the graphs of unitigs created by hifiasm to assemble contigs using Bandage (Wick et al., 2015, Bioinformatics 31, 3350-3352). Unitigs are uniquely assembleable subsets of overlapping fragments with no competing choices in terms of internal overlaps (https://wgs-assembler dot sourceforge dot net/wiki/index.php/Celera_Assembler_Terminology). The miR-430 locus is represented in a single graph of unitigs, which includes loops known to be at the origin of circular contigs. Thus, the single linear contig containing the miR-430 locus was used for further analysis and the circular contig containing miR-430 genes was excluded.

To estimate miR-430 copy number within HiFi reads and contigs, a non-redundant set of miR-430 mature sequences were extracted from miRBase (90) identified by dre-miR-430a-5p, dre-miR-430a-X-5p, dre-miR-430b-5p, and dre-miR-430c-5p. To normalize copy number in HiFi reads, 9 single-copy orthologs (SCO) were manually selected from the list obtained after executing the example SparQL query titled “Find most conservative single copy Danio rerio genes at the Vertebrata level” on OrthoDB (91). The coding exonic sequence of the SCOs tgfa (ENSDARG00000053939), flii (ENSDARG00000059701), trabd (ENSDARG00000010445), hhip (ENSDARG00000060397), smox (ENSDARG00000036967), pxk (ENSDARG00000063195), mtum (ENSDARG00000074505), trabd (ENSDARG00000010445), strap (ENSDARG00000007405) were extracted from Ensembl release 104 (Cunningham et al., Nucleic Acids Research 50, D988-D995 (2021)). To take into account the difference in length among 4 miRNA and 76 SCO exon sequences, a rate of 1 mutation per 20 nucleotides was allowed in retained alignment obtained using parasail (Daily, 2016, BMC Bioinformatics 17, 81) with the ‘sw_trace_striped_16′ function in a custom Python script. To avoid mapping sequences multiple times on the same locus, the full match (miR-430) or nucleotide (SCO exon) were masked from the target sequence after a match was identified. The normalized miR-430 copy numbers were calculated using the formula (miR-430 copy-number in reads/(SCO copy-number in reads/SCO copy-number in genome)). The same alignment approach was employed to count miR-430 copy-number in all assembled contigs.

CAGE-seq and Nanog ChIP-seq reads were mapped to the assembled contigs using STAR (Dobin et al., Bioinformatics 29, 15-21 (2012)) with the parameters ‘--alignEndsType EndToEnd’, ‘--outFilterMultimapNmax 10000′. ‘--outMultimapperOrder Random’, --scoreGap -8′, ‘--scoreGapNoncan 0’, ‘--scoreGapGCAG 0’, and ‘--scoreGapATAC 0’ effectively allowing long non-intronic gaps. BedGraph coverages were obtained using GeneAbacus (https://github.com/vejnar/GeneAbacus) with the parameters ‘--profile_type all-slice’, ‘--profile_multi 10000’, and ‘--profile_norm’. For Nanog ChIP single-end data, read coverage was extended using ‘--profile_extension length 184′ (Miao et al., 2022, Molecular Cell 82, 986-1002, e1009). To distinguish unique from multi mapper reads, reads were re-aligned on the contigs using minimap2 (Li, 2021, Bioinformatics 37, 4572-4574) with the parameters ‘-x map-hifi’ and --secondary-=ves’. A custom Python script generated BED files including primary and supplementary alignments as linked features while secondary alignment were attributed a specific score. Finally, CAGE-seq (BedGraph), Nanog ChIP-seq (BedGraph), miR-430 parasail alignments (BED), and read minimap2 alignments (BED) were plotted on contig track using pyGenomeTracks (Lopez-Delisle et al., 2020, Bioinformatics 37, 422-423).

### Pol II-pSer5 ChIP-Seq and Analysis

Dechorionated embryos were injected with 0.2 ng of α-amanitin at the 1-cell stage or left uninjected for the WT control. Chromatin immunoprecipitation (ChIP) was performed on α-amanitin injected and WT embryos, as described. Briefly, 1,000 embryos were collected at 4 hpf, fixed with 1.9% PFA at RT for 15 min, then quenched with 0.125 M glycine for 5 min at RT, washed 3× with cold PBS, flash frozen in liquid nitrogen, and stored at −80° C. until use. Samples were homogenized and lysed with cell lysis buffer (10 mM Tris-HCl pH 7.5, 10 mM NaCl, 0.5% IGEPAL, 1× protease inhibitors (Roche, catolog no. 11873580001)) on ice for 15 min. Nuclei were precipitated at 3,500 rpm at 4° C. for 5 min then lysed in nuclei lysis buffer (50 mM Tris-HCl pH 7.5, 10 mM EDTA, 1% SDS, protease inhibitors) on ice for 10 min. Next, samples were diluted with two volumes of IP dilution buffer (16.7 mM Tris-HCl pH 7.5, 167 mM NaCl, 1.2 mM EDTA, 0.01% SDS, protease inhibitors) and sonicated (Bioruptor Pico sonication device, Diagenode; sonication cycle: 30 seconds ON and 30 seconds OFF; 15 cycles of sonication, rest 15 minutes on ice, then 15 cycles of sonication). Triton X-100 was added to the sonicated chromatin (80 μl 10% Triton X-100 per 1 mL of sonicated chromatin) before centrifugation (10 minutes at 14,000 rpm at 4° C.). Subsequently, 5% of the supernatant representing the input was taken and stored at −80° C. Next, 25 μL of Protein G Dynabeads that were pre-incubated (overnight at 4° C.) with 4 μg of Pol II pSer5 antibody (Abcam, ab5131) were washed three times, added to the supernatant, and incubated overnight at 4° C. Beads were then washed 5× with RIPA wash buffer (50 mM HEPES pH 7.6, 1 mM EDTA, 0.7% DOC, 1% IGEPAL, 0.5 M LiCl) and 2× with TBS (Tris-buffered saline; 50 mM Tris pH 7.5, 150 mM NaCl), and eluted with elution buffer (50 mM NaHCO₃, 1% SDS) at 65° C. for 15 minutes with occasional vortexing. Input and ChIP samples were reverse-crosslinked by incubating at 65° C. overnight, treated with RNase A (0.33 μg/μL) for 2 hours at 37° C., then with Proteinase K (0.2 μg/μL) for 2 hours at 55° C., and purified. Libraries were prepared (Illumina TruSeq protocol) and sequenced (Illumina NovaSeq 6000 System, pair-end) at the Yale Center for Genome Analysis.

Sequencing data were managed using LabxDB seq (Vejnar et al., Bioinformatics 36, 4530-4531 (2020)) and mapped using LabxPipe (https://github dot com/vejnar/LabxPipe). Raw reads were adapter trimmed using ReadKnead 20 (https://github dot com/vejnar/ReadKnead) and mapped to the zebrafish GRCz11 genome sequence (Yates et al., Nucleic Acids Research 48, D682-D688 (2019)) using Bowtie2 (option: --no-unal, --no-discordant, --no-mixed, version: 2.4.5) (Langmead et al., Nature Methods 9, 357-359 (2012)). Usable reads (deduplicated and uniquely mapped with MAPQ≥30) were determined using samtools (Li et al., Bioinformatics 25, 2078-2079 (2009)) and used for the downstream analysis. Genomic tracks were created using the ‘trackhub’ option of LabxPipe which computed fragment genomic coverage normalized to the total fragments per million fragments using GeneAbacus (https://github dot com/vejnar/GeneAbacus) for ChIP-seq data generated here and for data from previous publications, including: Nanog ChIP-seq at 3.5 hpf (Xu et al., Developmental cell 22, 625-638 (2012)); Pan-Pol II ChIP-seq and Omnig-ATAC-seq at 4 hpf (Miao et al., Molecular Cell 82, 986-1002, e1009 (2022)). Line plots and heatmaps were created at single nucleotide resolution using deeptools (Ramirez et al., Nucleic Acids Res 42, W187-191 (2014)). Pol II-pSer5 ChIP-seq data were plotted at the following regions: across the gene body of zygotic genes (Chan et al. Developmental Cell 49, 867-881.e868 (2019)) with 2 kb flanking the transcription start site (TSS) and transcription end site (TES) and ranked by WT Pol II pSer5 ChIP-seq signal; at active enhancers as well as at Nanog-bound accessible regions previously defined (Miao et al., Molecular Cell 82, 986-1002.e1009 (2022)). For active enhancers, plots were centered at ATAC-seq peaks and ranked by WT Pol II pSer5 ChIP-seq signal. For Nanog-bound accessible regions, plots were centered at Nanog ChIP-seq peaks and ranked by Nanog ChIP-seq signal.

### Example 6: Gene Labeling Using CRISPR System Components

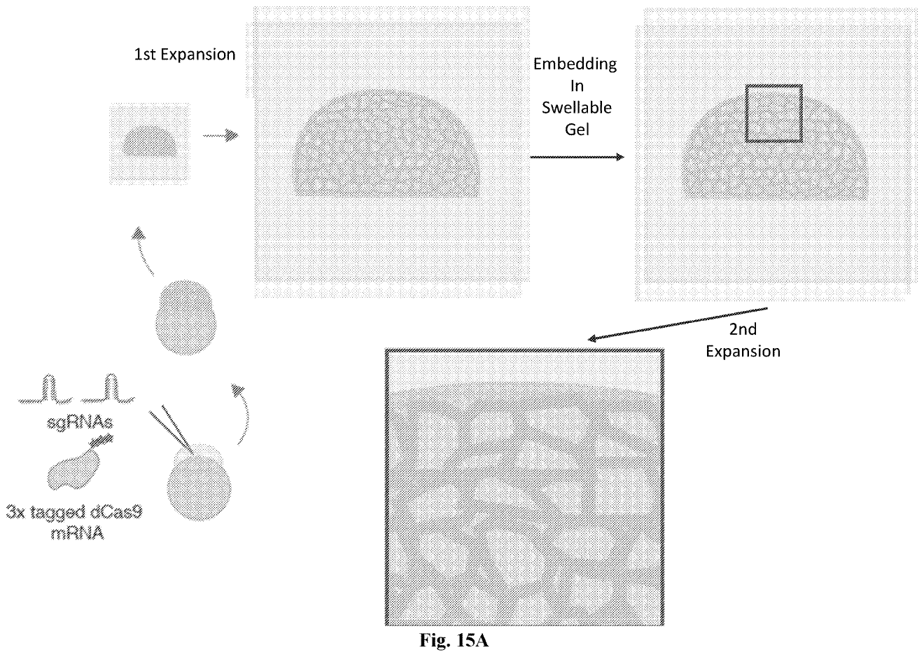

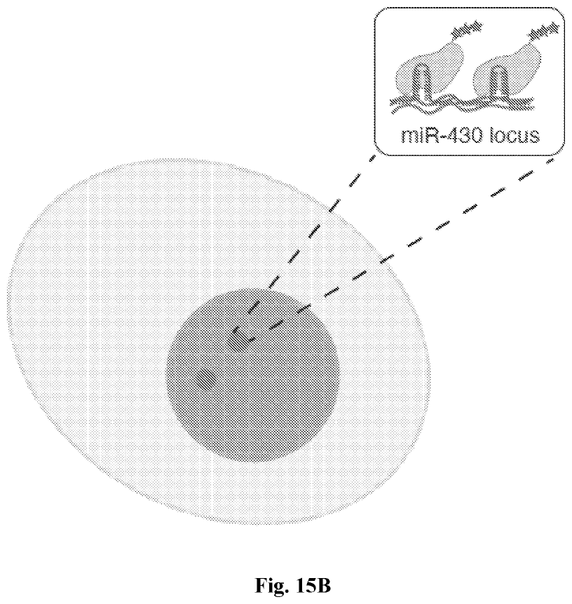

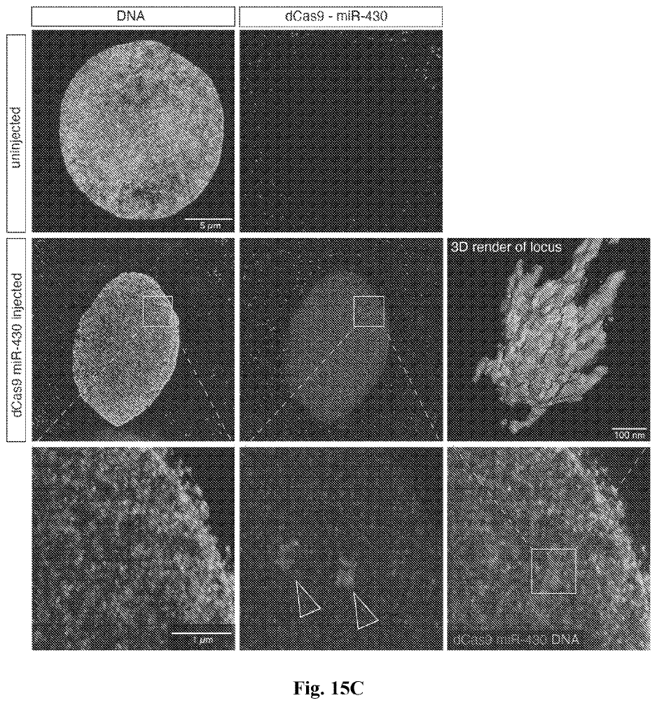

Referring to FIGS. 15A-15C, a system according to some embodiments herein, which uses single guide RNAs (sgRNAs) and Cas proteins to specifically label genes, was developed.

Referring to FIG. 15A-15B, embryos were injected with sgRNAs and mRNA encoding a catalytically inactive form of Cas9 (dCas9) targeting the miR-430 locus. At 3 hours post fertilization, embryos were fixed and subjected to ChromExM described in Examples 1 through 5. The first expansion and the second expansion were both about 4.5×.

Referring to FIG. 15C, dCas9 was detected by antibody staining in expanded cells injected with the dCas9 and the sgRNA, and the signal was absent in embryos not injected with dCas9. Two distinct foci of dCas9 signal are present in injected embryos (arrowheads), consistent with the two alleles of the miR-430 locus. Quantitative characteristics of these loci can then be extracted through 3D image analysis and renderings of the locus.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. A method of acquiring a microscopic image of a biological sample, the method comprising:
embedding the biological sample in a first swellable hydrogel;
expanding the first swellable hydrogel and the biological sample embedded therein to obtain a first expanded biological sample;
embedding the first expanded biological sample in a second swellable hydrogel;
labeling a DNA molecule in a chromatin in the biological sample with a first dye;
expanding the second swellable hydrogel and the first expanded biological sample embedded therein to obtain a second expanded biological sample; and
acquiring a microscopic image of the second expanded biological sample with light microscopy technology, wherein the acquired microscopic image includes the labeled chromatin.

2. The method of claim 1, wherein the first swellable hydrogel or the second swellable hydrogel is a polyacrylamide-based hydrogel.

3. The method of claim 1, wherein embedding the biological sample in the first swellable hydrogel comprises crosslinking the biological sample to the first swellable hydrogel.

4. The method of claim 1, wherein at least one of the following applies:
a first linear expansion factor of the biological sample in the expansion of the first swellable hydrogel is 3 or more,
a second linear expansion factor of the first expanded biological sample in the expansion of the second swellable hydrogel is 3 or more,
a combined linear expansion factor of the biological sample in the expansions of the first swellable hydrogel and the second swellable hydrogel is 10 or more.

5. (canceled)

6. The method of claim 1, wherein labeling the DNA molecule in the chromatin with the first dye comprises attaching the first dye to the DNA molecule via a covalent linker.

7. The method of claim 1, further comprising: introducing into the DNA molecule in the chromatin a modified nucleoside for attaching the first dye covalently,
optionally wherein the modified nucleoside is introduced into the DNA molecule metabolically.

8. (canceled)

9. The method of claim 7, wherein the modified nucleoside comprises 2′-deoxy-2′-fluoro-5-ethynyluridine (F-ara-EdU), 5-ethynyl-2′-deoxyuridine (EdU), 5-Ethynyl-2′-deoxycytidine (5-EdC), bromodeoxyuridine (BrdU), or azidomethyl-2′-deoxyuridine (AmdU).

10. The method of claim 1, wherein the first dye is attached to the DNA molecule using a click reaction.

11. The method of claim 1, wherein the first dye comprises an azide group and the DNA comprises an alkyne group or wherein the first dye comprises an alkyne group and the DNA comprises an azide group, and wherein the covalent linker is formed by a click reaction between the azide group and the alkyne group.

12. The method of claim 1, further comprising labeling a protein or an RNA molecule associated with the chromatin, wherein the microscopic image includes the labeled protein or the labeled RNA molecule.

13. The method of claim 12, wherein at least one of the following applies:
the protein is labeled, and the protein comprises a histone, a transcription factor, a polymerase, a nuclease, or DNA-binding portions thereof;
the protein is labeled by an antibody;
the RNA molecule is labeled, and the RNA molecule is labeled by a second dye;
the RNA molecule is labeled and is a transcription product of the DNA molecule in the chromatin.

14-16. (canceled)

17. The method of claim 1, which detects one or more sequences in the DNA molecule.

18. The method of claim 17, wherein the method comprising labeling the one or more sequences in the DNA molecule with a probe that binds to the one or more sequences specifically.

19. The method of claim 18, wherein the probe comprises a protein or a nucleic acid,
optionally wherein the probe comprises:
a nucleic acid that binds to the one or more sequences specifically; and
a protein that binds to the nucleic acid, or a protein that binds to a complex formed by the nucleic acid and the one or more sequences of the DNA molecule.

20. (canceled)

21. The method of claim 18, wherein the method directly visualizes the one or more sequences using a hybridization chain reaction (HCR) in situ hybridization method,
optionally wherein the probe comprises:
a single guide RNA (sgRNA) that binds to the one or more sequences specifically; and
a Cas protein that binds to a complex formed by the sgRNA and the one or more sequences,
optionally wherein the Cas is a catalytically dead Cas protein.

22-23. (canceled)

24. A method of evaluating effect of a treatment of a biological sample, the method comprising:
preparing a first biological sample and a second biological sample;
subjecting the first biological sample to the treatment without subjecting the second biological sample to the treatment;
acquiring a first microscopic image of the first biological sample and a second microscopic image of the second biological sample with the method of claim 1;
comparing the first microscopic image with the second microscopic image.

25. The method of claim 23, wherein the treatment comprises administration of a compound to a subject and wherein the biological sample is within the subject.

26. The method of claim 24, wherein at least one of the following applies:
comparing the first microscopic image with the second microscopic image comprises comparing a chromatin structure, a transcription activity, a DNA replication activity, a DNA damage, a DNA repair, or a DNA-protein interaction between the first microscopic image and the second microscopic image,
the first biological sample and the second biological sample are samples obtained from a subject, and the method evaluates the effect of the administration of a pharmaceutical compound to the first biological sample.

27. (canceled)
